SEQUENCE LISTING (78 sequences)

SEQ ID NO: 1 - protein, 277 aa, Homo sapiens
  SIGNAL 1..20
  PEPTIDE 21..277
  DOMAIN 21..193: note = extracellular domain
  DOMAIN 194..215: note = transmembrane domain
  DOMAIN 216..277: note = intracellular domain
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 2 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3 - protein, 448 aa, synthetic construct
  REGION 1..448: note = chimeric antibody with mouse variable domain, human constantdomain
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
EVQLQQSGPELEKPGASVKMSCKASGYSFTGYNMNWVKQSNGKSLEWIGNIDPYYGNTNYNQKFKGKATLTVDKSSSTAYLQLKSLTSEDSAVYYCARLGLQLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 219 aa, synthetic construct
  REGION 1..219: note = chimeric antibody with mouse variable domain and human constantdomain
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DVVMTQTPLSLPVSLGDQASISCRSSQSLVHSNGNTYLHWYLQKPGQSPNLLIYKLTNRFFGVPDRFSGSGSGTDFTLKISRVEAEDLGVYFCSQSIHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 219 aa, synthetic construct
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DIVMTQTPLSLPVTPGEPASISCRSSQSLVHSNGNTYLHWYLQKPGQSPQLLIYKLTNRFFGVPDRFSGSGSGTDFTLKISRVEAEDVGVYYCSQSIHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 448 aa, synthetic construct; identical to SEQ ID NO: 5
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3

SEQ ID NO: 8 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 219 aa, synthetic construct
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DIVMTQTPLSLPVTPGEPASISCRSSQSLVHSNANTYLHWYLQKPGQSPQLLIYKLTNRFFGVPDRFSGSGSGTDFTLKISRVEAEDVGVYYCSQSIHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 219 aa, synthetic construct; identical to SEQ ID NO: 6
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3

SEQ ID NO: 12 - protein, 448 aa, synthetic construct; identical to SEQ ID NO: 10
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3

SEQ ID NO: 13 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEDGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 448 aa, synthetic construct
  REGION 1..448: note = humanized antibody with mouse CDRs and human framework andconstant regions
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEEGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 448 aa, synthetic construct
  REGION 1..448: note = humanized antibody with mouse CDRs and human framework andconstant regions
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEEGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 448 aa, synthetic construct
  REGION 1..448: note = humanized antibody with mouse CDRs and human framework andconstant regions
  DOMAIN 1..119: note = variable domain
  REGION 31..35: note = MISC_FEATURE - CDRH1
  REGION 50..66: note = MISC_FEATURE - CDRH2
  REGION 99..108: note = MISC_FEATURE - CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPDLLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 446 aa, synthetic construct
  REGION 1..446: note = chimeric antibody with mouse variable domain and human constantdomain
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLQQSAAELARPGASVKMSCKASGYTFTDLSMHWIKQRPGQGLEWIGYITPSSGYTAYNQKFKGKTTLTADKSSSTAYMQLSSLTSEDSAVYYCARLILQRGAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 218 aa, synthetic construct
  REGION 1..218: note = chimeric antibody with mouse variable domain and human constantdomain
  DOMAIN 1..111: note = variable domain
  REGION 24..38: note = MISC_FEATURE - CDRL1
  REGION 54..60: note = MISC_FEATURE - CDRL2
  REGION 93..101: note = MISC_FEATURE - CDRL3
DIVLTQSPASLAVSLGQRATISCRASKNVDSYGNSFMHWYQQKPGQPPKLLIYRASNLESGIPARFGGSGSGTDFTLTINPVEADDVATYYCQQSNEDPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWIKQRPGQGLEWIGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 218 aa, synthetic construct
  REGION 1..218: note = humanized antibody with mouse CDRs and human framework andconstant regions
  DOMAIN 1..111: note = variable domain
  REGION 24..38: note = MISC_FEATURE - CDRL1
  REGION 54..60: note = MISC_FEATURE - CDRL2
  REGION 93..101: note = MISC_FEATURE - CDRL3
DIVMTQSPDSLAVSLGERATINCRASKNVDSYGNSFMHWYQQKPGQPPKLLIYRASNLESGVPDRFSGSGSGTDFTLTISSLQAEDVAVYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 446 aa, synthetic construct; identical to SEQ ID NO: 25
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3

SEQ ID NO: 28 - protein, 218 aa, synthetic construct
  REGION 1..218: note = humanized antibody with mouse CDRs and human framework andconstant regions
  DOMAIN 1..111: note = variable domain
  REGION 24..38: note = MISC_FEATURE - CDRL1
  REGION 54..60: note = MISC_FEATURE - CDRL2
  REGION 93..101: note = MISC_FEATURE - CDRL3
DIVLTQSPDSLAVSLGERATISCRASKNVDSYGNSFMHWYQQKPGQPPKLLIYRASNLESGVPDRFSGSGSGTDFTLTISSLQAEDVAVYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 218 aa, synthetic construct
  REGION 1..218: note = humanized antibody with mouse CDRs and human framework andconstant regions
  DOMAIN 1..111: note = variable domain
  REGION 24..38: note = MISC_FEATURE - CDRL1
  REGION 54..60: note = MISC_FEATURE - CDRL2
  REGION 93..101: note = MISC_FEATURE - CDRL3
DIVLTQSPDSLAVSLGERATINCRASKNVDSYGNSFMHWYQQKPGQPPKLLIYRASNLESGVPDRFSGSGSGTDFTLTISSLQAEDVAVYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEDGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 446 aa, synthetic construct
  REGION 1..446: note = humanized antibody with mouse CDRs and human framework andconstant regions
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEEGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 446 aa, synthetic construct
  REGION 1..446: note = humanized antibody with mouse CDRs and human framework andconstant regions
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 446 aa, synthetic construct
  REGION 1..446: note = humanized antibody with mouse CDRs and human framework andconstant regions
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEEGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 446 aa, synthetic construct
  REGION 1..446: note = humanized antibody with mouse CDRs and human framework andconstant regions
  DOMAIN 1..117: note = variable domain
  REGION 31..35: note = MISC_FEATURE - CDRH1
  REGION 50..66: note = MISC_FEATURE - CDRH2
  REGION 99..106: note = MISC_FEATURE - CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPDLLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 451 aa, synthetic construct
  REGION 1..451: note = chimeric antibody with mouse variable domain and human constantdomain
  DOMAIN 1..122: note = variable domain
  REGION 31..35: note = MISC_FEATURE - CDRH1
  REGION 50..66: note = MISC_FEATURE - CDRH2
  REGION 99..111: note = MISC_FEATURE - CDRH3
QVQFQQSGAELARPGASVKLSCKASGYTFTSYWMQWVKQRPGQGLEWIGTIYPGDGDSRYNQKFKGKALLTADKSSSIAYMQLNSLASEDSAVYFCARFSLYDGYPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 219 aa, synthetic construct
  REGION 1..219: note = chimeric antibody with mouse variable domain and human constantdomain
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DVVMTQTPLSLPVSLGDQASISCRSSQSLVHRNGNTYLHWYLQKPGQSPKLLIYRVSNRFSGVPDRFSGSGSGTDFTLKISRVEAEDLGIYFCSQSTHFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 451 aa, synthetic construct
  REGION 1..451: note = humanized antibody with mouse CDRs and human framework andconstant regions
  DOMAIN 1..122: note = variable domain
  REGION 31..35: note = MISC_FEATURE - CDRH1
  REGION 50..66: note = MISC_FEATURE - CDRH2
  REGION 99..111: note = MISC_FEATURE - CDRH3
QVQFVQSGAEVKKPGSSVKVSCKASGYTFTSYWMQWVRQAPGQGLEWMGTIYPGDGDSRYNQKFKGKALLTADKSTSTAYMELSSLRSEDTAVYYCARFSLYDGYPYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 219 aa, synthetic construct
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DIVMTQSPLSLPVTPGEPASISCRSSQSLVHRNGNTYLHWYLQKPGQSPQLLIYRVSNRFSGVPDRFSGSGSGTDFTLKISRVEAEDVGVYYCSQSTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 451 aa, synthetic construct
  REGION 1..451: note = humanized antibody with mouse CDRs and human framework andconstant regions
  DOMAIN 1..122: note = variable domain
  REGION 31..35: note = MISC_FEATURE - CDRH1
  REGION 50..66: note = MISC_FEATURE - CDRH2
  REGION 99..111: note = MISC_FEATURE - CDRH3
QVQFVQSGAEVKKPGSSVKVSCKASGYTFTSYWMQWVRQAPGQGLEWMGTIYPGDGDSRYNQKFKGKALLTADKSTSTAYMELSSLRSEDTAVYYCARFSLYEGYPYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 219 aa, synthetic construct; identical to SEQ ID NO: 43
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3

SEQ ID NO: 46 - protein, 451 aa, synthetic construct; identical to SEQ ID NO: 44
  REGION 1..451: note = humanized antibody with mouse CDRs and human framework andconstant regions
  DOMAIN 1..122: note = variable domain
  REGION 31..35: note = MISC_FEATURE - CDRH1
  REGION 50..66: note = MISC_FEATURE - CDRH2
  REGION 99..111: note = MISC_FEATURE - CDRH3

SEQ ID NO: 47 - protein, 219 aa, synthetic construct
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DIVMTQSPLSLPVTPGEPASISCRSSQSLVHRNANTYLHWYLQKPGQSPQLLIYRVSNRFSGVPDRFSGSGSGTDFTLKISRVEAEDVGVYYCSQSTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 451 aa, synthetic construct
  REGION 1..451: note = humanized antibody with mouse CDRs and human framework andconstant regions
  DOMAIN 1..122: note = variable domain
  REGION 31..35: note = MISC_FEATURE - CDRH1
  REGION 50..66: note = MISC_FEATURE - CDRH2
  REGION 99..111: note = MISC_FEATURE - CDRH3
QVQFVQSGAEVKKPGSSVKVSCKASGYTFTSYWMQWVRQAPGQGLEWMGTIYPGDSDSRYNQKFKGKALLTADKSTSTAYMELSSLRSEDTAVYYCARFSLYEGYPYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 219 aa, synthetic construct; identical to SEQ ID NO: 43
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3

SEQ ID NO: 50 - protein, 451 aa, synthetic construct; identical to SEQ ID NO: 48
  REGION 1..451: note = humanized antibody with mouse CDRs and human framework andconstant regions
  DOMAIN 1..122: note = variable domain
  REGION 31..35: note = MISC_FEATURE - CDRH1
  REGION 50..66: note = MISC_FEATURE - CDRH2
  REGION 99..111: note = MISC_FEATURE - CDRH3

SEQ ID NO: 51 - protein, 219 aa, synthetic construct; identical to SEQ ID NO: 47
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3

SEQ ID NO: 52 - protein, 442 aa, synthetic construct
  REGION 1..442: note = chimeric antibody with mouse variable domain and human constantdomain
  DOMAIN 1..113: note = variable domain
  REGION 31..35: note = MISC_FEATURE - CDRH1
  REGION 50..66: note = MISC_FEATURE - CDRH2
  REGION 99..102: note = MISC_FEATURE - CDRH3
EVLLQQSGPELVKPGASVKIPCKASGYKFTDYNMDWVKQSHGKSLEWIGDINPKNGGTIYNLKFKGKATLTVDMSSSTAYMELRSLTSEDTAVYYCVRRMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 214 aa, synthetic construct
  REGION 1..214: note = chimeric antibody with mouse variable domain and human constantdomain
  DOMAIN 1..107: note = variable domain
  REGION 24..34: note = MISC_FEATURE - CDRL1
  REGION 50..56: note = MISC_FEATURE - CDRL2
  REGION 89..97: note = MISC_FEATURE - CDRL3
DIQMTQTTSSLSASLGDRVTISCRASQDISNYLNWYQQKPDGTVKLLIYYISRLHSGVPSRFRGSGSGTDFSLTISNLEQEDIATYFCQQGNTLPYTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 442 aa, synthetic construct
  REGION 1..442: note = humanized antibody with mouse CDRs and human framework andconstant regions
  DOMAIN 1..113: note = variable domain
  REGION 31..35: note = MISC_FEATURE - CDRH1
  REGION 50..66: note = MISC_FEATURE - CDRH2
  REGION 99..102: note = MISC_FEATURE - CDRH3
QVQLVQSGAEVKKPGASVKVSCKASGYTFTDYNMDWVRQAPGQGLEWMGDINPKNGGTIYNLKFKGRVTLTTDTSTSTAYMELRSLRSDDTAVYYCVRRMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 214 aa, synthetic construct
  REGION 1..214: note = humanized antibody with mouse CDRs and human framework andconstant regions
  DOMAIN 1..107: note = variable domain
  REGION 24..34: note = MISC_FEATURE - CDRL1
  REGION 50..56: note = MISC_FEATURE - CDRL2
  REGION 89..97: note = MISC_FEATURE - CDRL3
DIQMTQSPSSLSASVGDRVTITCRASQDISNYLNWYQQKPGKVPKLLIYYISRLHSGVPSRFSGSGSGTDFTLTISSLQPEDVATYYCQQGNTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 442 aa, synthetic construct
  REGION 1..442: note = humanized antibody with mouse CDRs and human framework andconstant regions
  DOMAIN 1..113: note = variable domain
  REGION 31..35: note = MISC_FEATURE - CDRH1
  REGION 50..66: note = MISC_FEATURE - CDRH2
  REGION 99..102: note = MISC_FEATURE - CDRH3
QVQLVQSGAEVKKPGASVKVSCKASGYTFTDYNMDWVRQAPGQGLEWMGDINPKNAGTIYNLKFKGRVTLTVDTSTSTAYMELRSLRSDDTAVYYCVRRMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 214 aa, synthetic construct; identical to SEQ ID NO: 55
  REGION 1..214: note = humanized antibody with mouse CDRs and human framework andconstant regions
  DOMAIN 1..107: note = variable domain
  REGION 24..34: note = MISC_FEATURE - CDRL1
  REGION 50..56: note = MISC_FEATURE - CDRL2
  REGION 89..97: note = MISC_FEATURE - CDRL3

SEQ ID NO: 58 - protein, 441 aa, synthetic construct
  REGION 1..441: note = chimeric antibody with mouse variable domain and human constantdomain
  DOMAIN 1..112: note = variable domain
  REGION 31..35: note = MISC_FEATURE - CDRH1
  REGION 50..66: note = MISC_FEATURE - CDRH2
  REGION 99..101: note = MISC_FEATURE - CDRH3
EVQLVESGGGLVKPGGSLKLSCAASGFTFSSYAMSWVRQSPEKRLEWVAEISGGGSYTYYPDTVTGRFTISRDNAKNTLYLEMSSLRSEDTAMYYCASRAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 219 aa, synthetic construct
  REGION 1..219: note = chimeric antibody with mouse variable domain and human constantdomain
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DVLMTQTPLSLPVSLGDQASISCRSSQSIVHSNGNTYFEWYLQKPGQSPKLLIYKVSNRFSGVPDRFSGSGSGTDFTLKISRVEAEDLGVYYCFQGSHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 441 aa, synthetic construct
  REGION 1..441: note = humanized antibody with mouse CDRs and human framework andconstant regions
  DOMAIN 1..112: note = variable domain
  REGION 31..35: note = MISC_FEATURE - CDRH1
  REGION 50..66: note = MISC_FEATURE - CDRH2
  REGION 99..101: note = MISC_FEATURE - CDRH3
EVQLVESGGGLVKPGGSLRLSCAASGFTFSSYAMSWVRQAPGKGLEWVSEISGGGSYTYYPDTVTGRFTISRDNAKNSLYLQMNSLRAEDTAVYYCASRAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 441 aa, synthetic construct
  REGION 1..441: note = humanized antibody with mouse CDRs and human framework andconstant regions
  DOMAIN 1..112: note = variable domain
  REGION 31..35: note = MISC_FEATURE - CDRH1
  REGION 50..66: note = MISC_FEATURE - CDRH2
  REGION 99..101: note = MISC_FEATURE - CDRH3
EVQLVESGGGLVKPGGSLRLSCAASGFTFSSYAMSWVRQAPGKGLEWVSEISGGGSYTYYPDTVTGRFTISRDNAKNSLYLQMNSLRAEDTAVYYCASRAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 219 aa, synthetic construct
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DIVMTQTPLSLPVTPGEPASISCRSSQSIVHSNGNTYFEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 441 aa, synthetic construct; identical to SEQ ID NO: 60
  REGION 1..441: note = humanized antibody with mouse CDRs and human framework andconstant regions
  DOMAIN 1..112: note = variable domain
  REGION 31..35: note = MISC_FEATURE - CDRH1
  REGION 50..66: note = MISC_FEATURE - CDRH2
  REGION 99..101: note = MISC_FEATURE - CDRH3

SEQ ID NO: 64 - protein, 219 aa, synthetic construct
  REGION 1..219: note = humanized antibody with mouse CDRs and human framework andconstant regions
  DOMAIN 1..112: note = variable domain
  REGION 24..39: note = MISC_FEATURE - CDRL1
  REGION 55..61: note = MISC_FEATURE - CDRL2
  REGION 94..102: note = MISC_FEATURE - CDRL3
DIVMTQTPLSLPVTPGEPASISCRSSQSIVHSQGNTYFEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEDGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGDSVFLFPPKPKDTLMISRTPEVTCVVVDVSHEEGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPDLLGGDSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSHEEGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 329 aa, synthetic construct
  REGION 1..329: note = variant of human IgG1f constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPDLLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 107 aa, Homo sapiens [sequence omitted]

SEQ ID NO: 78 - protein, 17 aa, Homo sapiens
MRAWIFFLLCLAGRALA

(19) United States
(12) Patent Application Publication
(10) Pub. No.: US 2024/0336695 A1
(43) Pub. Date: Oct. 10, 2024

(54) ANTIBODIES TO CD40

(71) Applicant: BRISTOL-MYERS SQUIBB COMPANY, Princeton, NJ (US)

(72) Inventors: Bryan C. BARNHART, Vancouver (CA); Brigitte DEVAUX, Palo Alto, CA (US); Aaron P. YAMNIUK, Lawrenceville, NJ (US); Shannon L. OKADA, Seattle, WA (US); Brenda L. STEVENS, Seattle, WA (US)

(21) Appl. No.: 18/640,480

(22) Filed: Apr. 19, 2024

Related U.S. Application Data
(63) Continuation of application No. 17/091,375, filed on Nov. 6, 2020.
Application No. 17/091,375 is a division of application No. 16/399,379, filed on Apr. 30, 2019, now Pat. No. 10,844,130.
Application No. 16/399,379 is a division of application No. 15/579,450, filed on Dec. 4, 2017, now abandoned.
(60) Provisional application No. 62/252,615, filed on Nov. 9, 2015.
(60) Provisional application No. 62/186,076, filed on Jun. 29, 2015.

(51) Int. Cl. C07K 16/28; A61K 39/00; A61P 31/12; A61P 35/00; A61P 35/02; A61P 35/04

(52) U.S. Cl. CPC C07K 16/2878; A61P 31/12; A61P 35/00; A61P 35/02; A61P 35/04; A61K 2039/505; C07K 2317/21; C07K 2317/24; C07K 2317/34; C07K 2317/52; C07K 2317/56; C07K 2317/565; C07K 2317/72; C07K 2317/73; C07K 2317/732; C07K 2317/734; C07K 2317/75; C07K 2317/92; C07K 2317/94

(57) ABSTRACT

Provided herein are agonistic antibodies, or antigen binding portions thereof, that bind to human CD40. Such antibodies optionally comprise Fc regions with enhanced specificity for FcγRIIb. The invention also provides methods of treatment of cancer or chronic infection by administering the antibodies of the invention to a subject in need thereof.

### CROSS REFERENCE TO RELATED APPLICATIONS

This application is a continuation of U.S. Ser. No. 17/091,375, filed Nov. 6, 2020, which is a divisional of U.S. patent application Ser. No. 16/399,379, filed Apr. 30, 2019, now U.S. Pat. No. 10,844,130, which is a divisional of U.S. patent application Ser. No. 15/579,450, filed Dec. 4, 2017, now abandoned, which claims priority to U.S. Provisional Application Nos. 62/252,615, filed Nov. 9, 2015, and 62/186,076, filed Jun. 29, 2015, the disclosures of which are incorporated herein by reference.

### SEQUENCE LISTING

The Sequence Listing filed electronically herewith is hereby incorporated by reference in its entirety (File Name: 20220906_SEQL_12581-US-CNT.xml; Date Created: 17 Apr. 2024; File Size: 209 KB).

### BACKGROUND

Recent research has revealed that human cancers and chronic infections may be treated with agents that modulate the patient's immune response to malignant or infected cells. See, e.g., Reck & Paz-Ares (2015) Semin. Oncol. 42:402. Agonistic anti-CD40 antibodies, such as CP-870893 and dacetuzumab (SGN-40) have been tried for treating cancer based on the belief that they may enhance such an immune response. See, e.g., Kirkwood et al. (2012) CA Cancer J. Clin. 62:309; Vanderheide & Glennie (2013) Clin. Cancer Res. 19:1035. Recent experiments in mice have revealed that anti-CD40 antibodies with enhanced specificity for the inhibitory Fc receptor FcγRIIb have increased anti-tumor efficacy. See, e.g., WO 2012/087928; Li & Ravetch (2011) Science 333:1030; Li & Ravetch (2012) Proc. Nat'l Acad. Sci USA 109:10966; Wilson et al. (2011) Cancer Cell 19:101; White et al. (2011) J Immunol. 187:1754.

The need exists for improved agonistic anti-human CD40 antibodies for treatment of cancer and chronic infections in human subjects. Such antibodies will preferably have enhanced specificity for the inhibitory Fc receptor FcγRIIb as compared to activating Fc receptors, and will exhibit enhanced anti-tumor and/or anti-infective activity.

### SUMMARY OF THE INVENTION

Provided herein are isolated humanized murine monoclonal antibodies that specifically bind to human CD40 (the mature sequence of SEQ ID NO: 1), optionally having modified Fc regions that enhance specificity for binding to FcγRIIb receptor. In certain embodiments, the invention relates to anti-huCD40 antibodies or antigen binding fragments thereof that compete for binding with, cross-block, or bind to the same epitope as, one or more of antibodies 12D6 (SEQ ID NOs: 3 and 4), 5F11 (SEQ ID NOs: 23 and 24), 8E8 (SEQ ID NOs: 40 and 41), 5G7 (SEQ ID NOs: 52 and 53), and 19G3 (SEQ ID NOs: 58 and 59), including chimeric, human or humanized antibodies.

In certain embodiments, the anti-human CD40 antibodies of the present invention, or antigen binding fragments thereof, bind at an epitope comprising or consisting of one or more sequences selected from the group consisting of WGCLLTAVHPEPPTACRE (residues 11-28 of SEQ ID NO: 1) (antibody 12D6), EPPTACREKQYLINS (residues 21-35 of SEQ ID NO: 1) (antibodies 12D6, 5G7 and 19G3), and ECLPCGESE (residues 58-66 of SEQ ID NO: 1) (antibody 5F11).

In some embodiments the antibody of the present invention comprises a heavy chain and a light chain, wherein the heavy chain comprises CDRH1, CDRH2 and CDRH3 sequences and the light chain comprises CDRL1, CDRL2 and CDRL3 sequences derived at least in part from the same mouse germline V region gene segments and J region gene segments as anti-huCD40 antibody 12D6, 5F11, 8E8, 5G7 or 19G3, as disclosed at Table 3. Specifically, the antibody may comprise CDR sequences derived from the same murine germlines as antibody 12D6 (heavy chain CDR sequences derived at least in part from murine V region germline VH1-39_01 and J region germline IGHJ4 and light chain CDR sequences derived at least in part from murine V region germline VK1-110_01 and J region germline IGKJ1), antibody 5F11 (heavy chain CDR sequences derived at least in part from murine V region germline VH1-4_02 and J region germline IGHJ3 and light chain CDR sequences derived at least in part from murine V region germline VK3-5_01 and J region germline IGKJ5), antibody 8E8 (heavy chain CDR sequences derived at least in part from murine V region germline VH1-80_01 and J region germline IGHJ2 and light chain CDR sequences derived at least in part from murine V region germline VK1-110_01 and J region germline IGKJ2), antibody 5G7 (heavy chain CDR sequences derived at least in part from murine V region germline VH1-18_01 and J region germline IGHJ4 and light chain CDR sequences derived at least in part from murine V region germline VK10⁻⁹⁶_01 and J region germline IGKJ2), or antibody 19G3 (heavy chain CDR sequences derived at least in part from murine V region germline VH5-9-4_01 and J region germline IGHJ3 and light chain CDR sequences derived at least in part from murine V region germline VK1-117_01 and J region germline IGKJ2).

In various embodiments the antibody of the present invention comprises a heavy chain and a light chain, wherein the heavy chain comprises CDRH1, CDRH2 and CDRH3 sequences and the light chain comprises CDRL1, CDRL2 and CDRL3 sequences selected from the group consisting of: the CDRs of antibody 12D6-03 wherein CDRH1, CDRH2 and CDRH3 comprise residues 31-35, 50-66 and 99-108, respectively, of SEQ ID NO:5 and CDRL1, CDRL2 and CDRL3 comprise residues 24-39, 55-61 and 94-102, respectively, of SEQ ID NO:6; the CDRs of antibody 12D6-22 wherein CDRH1, CDRH2 and CDRH3 comprise residues 31-35, 50-66 and 99-108, respectively, of SEQ ID NO:7 and CDRL1, CDRL2 and CDRL3 comprise residues 24-39, 55-61 and 94-102, respectively, of SEQ ID NO:9; the CDRs of antibody 12D6-23 wherein CDRH1, CDRH2 and CDRH3 comprise residues 31-35, 50-66 and 99-108, respectively, of SEQ ID NO:10 and CDRL1, CDRL2 and CDRL3 comprise residues 24-39, 55-61 and 94-102, respectively, of SEQ ID NO:11; the CDRs of antibody 12D6-24 wherein CDRH1, CDRH2 and CDRH3 comprise residues 31-35, 50-66 and 99-108, respectively, of SEQ ID NO:12 and CDRL1, CDRL2 and CDRL3 comprise residues 24-39, 55-61 and 94-102, respectively, of SEQ ID NO:9; the CDRs of antibody 5F11-17 wherein CDRH1, CDRH2 and CDRH3 comprise residues 31-35, 50-66 and 99-106, respectively, of SEQ ID NO:25 and CDRL1, CDRL2 and CDRL3 comprise residues 24-38, 54-60 and 93-101, respectively, of SEQ ID NO:26; the CDRs of antibody 5F11-23 wherein CDRH1, CDRH2 and CDRH3 comprise residues 31-35, 50-66 and 99-106, respectively, of SEQ ID NO:27 and CDRL1, CDRL2 and CDRL3 comprise residues 24-38, 54-60 and 93-101, respectively, of SEQ ID NO:28; the CDRs of antibody 5F11-45 wherein CDRH1, CDRH2 and CDRH3 comprise residues 31-35, 50-66 and 99-106, respectively, of SEQ ID NO:29 and CDRL1, CDRL2 and CDRL3 comprise residues 24-38, 54-60 and 93-101, respectively, of SEQ ID NO:30; the CDRs of antibody 8E8-56 wherein CDRH1, CDRH2 and CDRH3 comprise residues 31-35, 50-66 and 99-111, respectively, of SEQ ID NO:42 and CDRL1, CDRL2 and CDRL3 comprise residues 24-39, 55-61 and 94-102, respectively, of SEQ ID NO:43; the CDRs of antibody 8E8-62 wherein CDRH1, CDRH2 and CDRH3 comprise residues 31-35, 50-66 and 99-111, respectively, of SEQ ID NO:44 and CDRL1, CDRL2 and CDRL3 comprise residues 24-39, 55-61 and 94-102, respectively, of SEQ ID NO:45; the CDRs of antibody 8E8-67 wherein CDRH1, CDRH2 and CDRH3 comprise residues 31-35, 50-66 and 99-111, respectively, of SEQ ID NO:46 and CDRL1, CDRL2 and CDRL3 comprise residues 24-39, 55-61 and 94-102, respectively, of SEQ ID NO:47; the CDRs of antibody 8E8-70 wherein CDRH1, CDRH2 and CDRH3 comprise residues 31-35, 50-66 and 99-111, respectively, of SEQ ID NO:48 and CDRL1, CDRL2 and CDRL3 comprise residues 24-39, 55-61 and 94-102, respectively, of SEQ ID NO:49; the CDRs of antibody 8E8-71 wherein CDRH1, CDRH2 and CDRH3 comprise residues 31-35, 50-66 and 99-111, respectively, of SEQ ID NO:50 and CDRL1, CDRL2 and CDRL3 comprise residues 24-39, 55-61 and 94-102, respectively, of SEQ ID NO:51; the CDRs of antibody 5G7-22 wherein CDRH1, CDRH2 and CDRH3 comprise residues 31-35, 50-66 and 99-102, respectively, of SEQ ID NO:54 and CDRL1, CDRL2 and CDRL3 comprise residues 24-34, 50-56 and 89-97, respectively, of SEQ ID NO:55; the CDRs of antibody 5G7-25 wherein CDRH1, CDRH2 and CDRH3 comprise residues 31-35, 50-66 and 99-102, respectively, of SEQ ID NO:56 and CDRL1, CDRL2 and CDRL3 comprise residues 24-34, 50-56 and 89-97, respectively, of SEQ ID NO:57; the CDRs of antibody 19G3-11 wherein CDRH1, CDRH2 and CDRH3 comprise residues 31-35, 50-66 and 99-101, respectively, of SEQ ID NO:60 and CDRL1, CDRL2 and CDRL3 comprise residues 24-39, 55-61 and 94-102, respectively, of SEQ ID NO:62; and the CDRs of antibody 19G3-22 wherein CDRH1, CDRH2 and CDRH3 comprise residues 31-35, 50-66 and 99-101, respectively, of SEQ ID NO:63 and CDRL1, CDRL2 and CDRL3 comprise residues 24-39, 55-61 and 94-102, respectively, of SEQ ID NO:64.

In various embodiments the antibody of the present invention comprises a heavy chain comprising a variable domain selected from the group consisting of 12D6 (residues 1-119 of SEQ ID NO: 3), 5F11 (residues 1-117 of SEQ ID NO: 23), 8E8 (residues 1-122 of SEQ ID NO: 40), 5G7(residues 1-113 of SEQ ID NO: 52) and 19G3 (residues 1-112 of SEQ ID NO: 58) with constant regions comprising FcγRIIb-specific Fc region selected from the group consisting of IgG1f (SEQ ID NO: 65), SE (SEQ ID NO: 66), SELF (SEQ ID NO: 67), P238D (SEQ ID NO: 68), V4 (SEQ ID NO: 69), V4 D270E (SEQ ID NO: 70), V7 (SEQ ID NO: 71), V8 (SEQ ID NO: 72), V9 (SEQ ID NO: 73), V9 D270E (SEQ ID NO: 74), V11 (SEQ ID NO: 75), and V12 (SEQ ID NO: 76).

In some embodiments the antibody comprises specific heavy chain variable domains and light chain variable domains selected from the group consisting of 12D6-03 (residues 1-119 and 1-112 of SEQ ID NO:5 and SEQ ID NO:6, respectively), 12D6-22 (residues 1-119 and 1-112 of SEQ ID NO:7 and SEQ ID NO:9, respectively), 12D6-23 (residues 1-119 and 1-112 of SEQ ID NO:10 and SEQ ID NO:11, respectively), 12D6-24 (residues 1-119 and 1-112 of SEQ ID NO:12 and SEQ ID NO:9, respectively), 5F11-17 (residues 1-117 and 1-111 of SEQ ID NO:25 and SEQ ID NO:26, respectively), 5F11-23 (residues 1-117 and 1-111 of SEQ ID NO:27 and SEQ ID NO:28, respectively), 5F11-45 (residues 1-117 and 1-111 of SEQ ID NO:29 and SEQ ID NO:30), 8E8-56 (residues 1-122 and 1-112 of SEQ ID NO:42 and SEQ ID NO:43, respectively), 8E8-62 (residues 1-122 and 1-112 of SEQ ID NO:44 and SEQ ID NO:45, respectively), 8E8-67 (residues 1-122 and 1-112 of SEQ ID NO:46 and SEQ ID NO:47, respectively), 8E8-70 (residues 1-122 and 1-112 of SEQ ID NO:48 and SEQ ID NO:49), 8E8-71 (residues 1-122 and 1-112 of SEQ ID NO:50 and SEQ ID NO:51, respectively), 5G7-22 (residues 1-113 and 1-107 of SEQ ID NO:54 and SEQ ID NO:55, respectively), 5G7-25 (residues 1-113 and 1-107 of SEQ ID NO:56 and SEQ ID NO:57, respectively), 19G3-11 (residues 1-112 and 1-112 of SEQ ID NO:60 and SEQ ID NO:62, respectively), and 9G3-22 (residues 1-112 and 1-112 of SEQ ID NO:63 and SEQ ID NO:64, respectively). Any of these antibodies may further comprise a heavy chain constant region comprising an FcγRIIb-specific Fc region, said heavy chain constant region selected from the group consisting of IgG1f (SEQ ID NO: 65), SE (SEQ ID NO: 66), SELF (SEQ ID NO: 67), P238D (SEQ ID NO: 68), V4 (SEQ ID NO: 69), V4 D270E (SEQ ID NO: 70), V7 (SEQ ID NO: 71), V8 (SEQ ID NO: 72), V9 (SEQ ID NO: 73), V9 D270E (SEQ ID NO: 74), V11 (SEQ ID NO: 75), and V12 (SEQ ID NO: 76). Any of these antibodies may further comprise the light chain kappa constant region of SEQ ID NO: 77.

In specific embodiments, the antibody of the present invention comprises a humanized 12D6-24 antibody comprising a light chain of SEQ ID NO: 9 and a heavy chain selected from the group consisting of any of SEQ ID NOs: 13-22, or a humanized 5F111-45 antibody comprising a light chain of SEQ ID NO: 30 and a heavy chain selected from the group consisting of any of SEQ ID NOs: 31-39. Specific antibodies include 12D6-24 SE (SEQ ID NOs: 9 and 14), 12D6-24 SELF (SEQ ID NOs: 9 and 15), 12D6-24 P238D (SEQ ID NOs: 9 and 13), 12D6-24 V4 (SEQ ID NOs: 9 and 16), 12D6-24 V4 D270E (SEQ ID NOs: 9 and 17), 12D6-24 V8 (SEQ ID NOs: 9 and 18), 12D6-24 V9 (SEQ ID NOs: 9 and 19), 12D6-24 V9 D270E (SEQ ID NOs: 9 and 20), 12D6-24 V11 (SEQ ID NOs: 9 and 21), 12D6-24 V12 (SEQ ID NOs: 9 and 22), 5F11-45 SE (SEQ ID NOs: 30 and 31), 5F11-45 SELF (SEQ ID NOs: 30 and 32), 5F11-45 V4 (SEQ ID NOs: 30 and 33), 5F11-45 V4 D270E (SEQ ID NOs: 30 and 34), 5F11-45 V8 (SEQ ID NOs: 30 and 35), 5F11-45 V9 (SEQ ID NOs: 30 and 36), 5F11-45 V9 D270E (SEQ ID NOs: 30 and 37), 5F11-45 V11 (SEQ ID NOs: 30 and 38), and 5F11-45 V12 (SEQ ID NOs: 30 and 39), where sequences are provided for light and heavy chains, respectively.

In further embodiments the anti-huCD40 antibodies of the present comprise heavy and light chains sharing at least 80%, 85%, 90% and 95% sequence identity with the sequences of the heavy and light chains of 12D6-24 SE (SEQ ID NOs: 9 and 14), 12D6-24 SELF (SEQ ID NOs: 9 and 15), 12D6-24 P238D (SEQ ID NOs: 9 and 13), 12D6-24 V4 (SEQ ID NOs: 9 and 16), 12D6-24 V4 D270E (SEQ ID NOs: 9 and 17), 12D6-24 V8 (SEQ ID NOs: 9 and 18), 12D6-24 V9 (SEQ ID NOs: 9 and 19), 12D6-24 V9 D270E (SEQ ID NOs: 9 and 20), 12D6-24 V11 (SEQ ID NOs: 9 and 21), 12D6-24 V12 (SEQ ID NOs: 9 and 22), 5F11-45 SE (SEQ ID NOs: 30 and 31), 5F11-45 SELF (SEQ ID NOs: 30 and 32), 5F111-45 V4 (SEQ ID NOs: 30 and 33), 5F11-45 V4 D270E (SEQ ID NOs: 30 and 34), 5F111-45 V8 (SEQ ID NOs: 30 and 35), 5F111-45 V9 (SEQ ID NOs: 30 and 36), 5F111-45 V9 D270E (SEQ ID NOs: 30 and 37), 5F111-45 V11 (SEQ ID NOs: 30 and 38), or 5F111-45 V12 (SEQ ID NOs: 30 and 39).

In yet further embodiments the anti-huCD40 antibodies of the present comprise heavy and light chains consisting essentially of the sequences of the heavy and light chains of 12D6-24 SE (SEQ ID NOs: 9 and 14), 12D6-24 SELF (SEQ ID NOs: 9 and 15), 12D6-24 P238D (SEQ ID NOs: 9 and 13), 12D6-24 V4 (SEQ ID NOs: 9 and 16), 12D6-24 V4 D270E (SEQ ID NOs: 9 and 17), 12D6-24 V8 (SEQ ID NOs: 9 and 18), 12D6-24 V9 (SEQ ID NOs: 9 and 19), 12D6-24 V9 D270E (SEQ ID NOs: 9 and 20), 12D6-24 V11 (SEQ ID NOs: 9 and 21), 12D6-24 V12 (SEQ ID NOs: 9 and 22), 5F111-45 SE (SEQ ID NOs: 30 and 31), 5F11-45 SELF (SEQ ID NOs: 30 and 32), 5F11-45 V4 (SEQ ID NOs: 30 and 33), 5F11-45 V4 D270E (SEQ ID NOs: 30 and 34), 5F11-45 V8 (SEQ ID NOs: 30 and 35), 5F11-45 V9 (SEQ ID NOs: 30 and 36), 5F11-45 V9 D270E (SEQ ID NOs: 30 and 37), 5F111-45 V11 (SEQ ID NOs: 30 and 38), or 5F111-45 V12 (SEQ ID NOs: 30 and 39).

In some embodiments, anti-huCD40 antibodies of the present invention that comprise V4 or V9 Fc sequence variants further comprise the D270E sequence variant. Such antibodies include a humanized 12D6-24 V4 D270E (SEQ ID NOs: 9 and 17), 12D6-24 V9 D270E (SEQ ID NOs: 9 and 20), 5F11-45 V4 D270E (SEQ ID NOs: 30 and 34), and 5F11-45 V9 D270E (SEQ ID NOs: 30 and 37), where sequences are provided for light and heavy chains, respectively. In alternative embodiments, anti-human CD40 antibodies of the present invention include antibodies comprising heavy and light chains consisting essentially of the sequences of these heavy and light chains, or comprise heavy and light chains sharing at least 80%, 85%, 90% and 95% sequence identity with these sequences. In some embodiments, the anti-huCD40 antibodies of the present invention comprise modified Fc regions with greater specificity for binding to FcγRIIb as opposed to binding to activating receptors than antibodies with naturally occurring Fc regions. In certain embodiments the A/I ratio for the anti-huCD40 antibody of the present invention is less than 5, and in preferred embodiments, less than 1.

In some embodiments the anti-huCD40 antibody of the present invention comprises one or more heavy chains and one or more light chains, such as two heavy chains and two light chains.

The present invention further provides nucleic acids encoding the heavy and/or light chain variable regions, of the anti-CD40 antibodies of the present invention, or antigen binding fragments thereof, expression vectors comprising the nucleic acid molecules, cells transformed with the expression vectors, and methods of producing the antibodies by expressing the antibodies from cells transformed with the expression vectors and recovering the antibody.

The present invention also provides pharmaceutical compositions comprising anti-huCD40 antibodies of the present invention, or antigen binding fragments thereof, and a carrier.

The present invention provides a method of enhancing an immune response in a subject comprising administering an effective amount of an anti-huCD40 antibody of the present invention, or antigen binding fragment thereof, to the subject such that an immune response in the subject is enhanced. In certain embodiments, the subject has a tumor and an immune response against the tumor is enhanced. In another embodiment, the subject has a viral infection, e.g. a chronic viral infection, and an anti-viral immune response is enhanced.

The present invention also provides a method of inhibiting the growth of tumors in a subject comprising administering to the subject an anti-huCD40 antibody of the present invention, or antigen binding fragment thereof, such that growth of the tumor is inhibited.

The present invention further provides a method of treating cancer, e.g., by immunotherapy, comprising administering to a subject in need thereof a therapeutically effective amount an anti-huCD40 antibody of the present invention, or antigen binding fragment thereof, e.g. as a pharmaceutical composition, thereby treating the cancer. In certain embodiments, the cancer is bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer. In certain embodiments, the cancer is a metastatic cancer, refractory cancer, or recurrent cancer.

In certain embodiments, the methods of modulating immune function and methods of treatment described herein comprise administering an anti-huCD40 antibody of the present invention in combination with, or as a bispecific reagent with, one or more additional therapeutics, for example, an anti-PD1 antibody, an anti-PD-L1 antibody, an anti-LAG3 antibody, an anti-GITR antibody, an anti-OX40 antibody, an anti-CD73 antibody, an anti-TIGIT antibody, an anti-CD137 antibody, an anti-CD27 antibody, an anti-CSF-1R antibody, an anti-CTLA-4 antibody, a TLR agonist, or a small molecule antagonist of IDO or TGFβ. In specific embodiments, anti-huCD40 therapy is combined with anti-PD1 and/or anti-PD-L1 therapy, e.g. treatment with an antibody or antigen binding fragment thereof that binds to human PD1 or an antibody or antigen binding fragment thereof that binds to human PD-L1.

### BRIEF DESCRIPTION OF THE DRAWINGS

FIG. 1 shows the sequence of human IgG1f constant domain (SEQ ID NO: 65) renumbered 118-446 to better illustrate the Fc sequence variants disclosed herein (Table 4). Residues subject to variation are in bold, and the altered amino acid is provided in bold below the residue. The D270E substitution is underlined. A C-terminal lysine (K) residue has been removed in FIG. 1 and SEQ ID NO: 65, as well as all other heavy chain and heavy chain constant domain sequences disclosed in the Sequence Listing. However, in other embodiments, especially nucleic acid constructs encoding the heavy chains and heavy chain constant domains of the anti-huCD40 antibodies of the present invention, these sequences include an additional lysine residue at the C-terminus of the protein or nucleotides encoding the extra lysine at the 3′ end of the nucleic acid.

FIG. 2 is a Venn diagram illustrating the epitope groups (“bins”) on human CD40 bound by the antibodies of the present invention, as well as blocking of CD40L binding. Antibodies with overlapping ovals or circles compete for binding to human CD40, and antibodies falling within the rectangle block CD40L binding to human CD40.

FIGS. 3A and 3B show activation of dendritic cells, as measured by IL-6 secretion, by agonist anti-CD40 antibodies as a function of Fc sequence. See Example 7. A series of antibodies was constructed comprising an mAb 12D6-24 variable domain and various human IgG1f constant regions, including IgG1f, SE, SELF, P238D, V4, V8, V9 and V12 variants. FIG. 3A presents data obtained using cells from one donor, and FIG. 3B presents data obtained using cells from a different donor.

FIG. 4 shows activation of cells, as measured by cell surface CD54, by agonist anti-CD40 antibodies as a function of variable domain sequence. See Example 7. A series of antibodies was constructed comprising a human IgG1f-V12 constant region and variable domains from parental (murine) anti-CD40 mAbs 12D6, 5G7, 8E8, 19G3 and 5F11. Results are plotted as median fluorescence intensity (MFI) as a function of antibody concentration.

FIG. 5 shows percent FcγR binding by various antibodies of the present invention, including antibodies having D270 substitutions. See Example 8. Antibody names including “-sup” represent supernatants from antibody producing cells, whereas others are purified antibodies. Data are presented as percentages of a maximum receptor binding value for each combination of antibody and receptor, as measured in a ForteBio Octet system. See, e.g., Example 3. Each cluster of three bars represents, from left to right, binding to hCD32a/FcγRIIa-H131 (10 μM) (hatched bars), hCD32b/FcγRIIa-R131 (10 μM) (black bars), and hCD32b/FcγRIIb (1 μM) (white bars).

### DETAILED DESCRIPTION

The present invention provides isolated antibodies, particularly monoclonal antibodies, e.g., humanized or human monoclonal antibodies, that specifically bind to human CD40 (“huCD40”) and have agonist activity. Sequences are provided for various humanized murine anti-huCD40 monoclonal antibodies. In certain embodiments, the antibodies described herein are derived from particular murine heavy and light chain germline sequences and/or comprise particular structural features such as CDR regions comprising particular amino acid sequences. In other embodiments antibodies compete for CD40 binding with, or bind to the same epitope as, the anti-CD40 antibodies for which sequences are provided herein. In some embodiments the sequence of the heavy chain Fc region is modified to specifically enhance binding to FcγRIIb.

Further provided herein are methods of making such antibodies, immunoconjugates and bispecific molecules comprising such antibodies or antigen-binding fragments thereof, and pharmaceutical compositions formulated to contain the antibodies or fragments. Also provided herein are methods of using the antibodies for immune response enhancement, alone or in combination with other immunostimulatory agents (e.g., antibodies) and/or cancer or anti-infective therapies. Accordingly, the anti-huCD40 antibodies described herein may be used in a treatment in a wide variety of therapeutic applications, including, for example, inhibiting tumor growth and treating chronic viral infections.

### Definitions

In order that the present description may be more readily understood, certain terms are first defined. Additional definitions are set forth throughout the detailed description.

CD40 refers to “TNF receptor superfamily member 5” (TNFRSF5). Unless otherwise indicated, or clear from the context, references to CD40 herein refer to human CD40 (“huCD40”), and anti-CD40 antibodies refer to anti-human CD40 antibodies. Human CD40 is further described at GENE ID NO: 958 and MIM (Mendelian Inheritance in Man): 109535. The sequence of human CD40 (NP_001241.1), including 20 amino acid signal sequence, is provided at SEQ ID NO: 1.

CD40 interacts with CD40 ligand (CD40L), which is also referred to as TNFSF5, gp39 and CD154. Unless otherwise indicated, or clear from the context, references to CD40L herein refer to human CD40L (“huCD40L”). Human CD40L is further described at GENE ID NO: 959 and MIM: 300386. The sequence of human CD40L (NP_000065.1) is provided at SEQ ID NO: 2.

Unless otherwise indicated or clear from the context, the term “antibody” as used to herein may include whole antibodies and any antigen-binding fragments (i.e., “antigen-binding portions”) or single chains thereof. An “antibody” refers, in one embodiment, to a glycoprotein comprising at least two heavy (H) chains and two light (L) chains inter-connected by disulfide bonds, or an antigen binding fragment thereof. Each heavy chain is comprised of a heavy chain variable region (abbreviated herein as V_{H}) and a heavy chain constant region. In certain naturally occurring IgG, IgD and IgA antibodies, the heavy chain constant region is comprised of three domains, CH1, CH2 and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four framework regions (FRs), arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

Antibodies typically bind specifically to their cognate antigen with high affinity, reflected by a dissociation constant (K_{D}) of 10⁻⁷ to 10⁻¹¹ M or less. Any K_{D} greater than about 10⁻⁶ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 5×10⁻⁹ M or less, and most preferably between 10⁻⁸ M and 10⁻¹⁰ M or less, but does not bind with high affinity to unrelated antigens. An antigen is “substantially identical” to a given antigen if it exhibits a high degree of sequence identity to the given antigen, for example, if it exhibits at least 80%, at least 90%, preferably at least 95%, more preferably at least 97%, or even more preferably at least 99% sequence identity to the sequence of the given antigen. By way of example, an antibody that binds specifically to human CD40 might also cross-react with CD40 from certain non-human primate species (e.g., cynomolgus monkey), but might not cross-react with CD40 from other species, or with an antigen other than CD40.

Unless otherwise indicated, an immunoglobulin may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., human IgG1, exist in several allotypes, which differ from each other in at most a few amino acids. Unless otherwise indicated, antibodies of the present invention comprise the IgG1f constant domain (SEQ ID NO: 65). Unless otherwise indicated, “antibody” may include, by way of example, monoclonal and polyclonal antibodies; chimeric and humanized antibodies; human and non-human antibodies; wholly synthetic antibodies; and single chain antibodies.

The term “antigen-binding portion” or “antigen binding fragment” of an antibody, as used herein, refers to one or more fragments of an antibody that retain the ability to specifically bind to an antigen (e.g., human CD40). Examples of binding fragments encompassed within the term “antigen-binding portion/fragment” of an antibody include (i) a Fab fragment—a monovalent fragment consisting of the V_{L}, V_{H}, CL and CH1 domains; (ii) a F(ab′)₂ fragment—a bivalent fragment comprising two Fab fragments linked by a disulfide bridge at the hinge region; (iii) a Fd fragment consisting of the VH and CH1 domains; (iv) a Fv fragment consisting of the V_{L} and V_{H} domains of a single arm of an antibody, and (v) a dAb fragment (Ward et al., (1989) Nature 341:544-546) consisting of a V_{H} domain. An isolated complementarity determining region (CDR), or a combination of two or more isolated CDRs joined by a synthetic linker, may comprise and antigen binding domain of an antibody if able to bind antigen.

Single chain antibody constructs are also included in the invention. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion/fragment” of an antibody. These and other potential constructs are described at Chan & Carter (2010) Nat. Rev. Immunol. 10:301. These antibody fragments are obtained using conventional techniques known to those with skill in the art, and the fragments are screened for utility in the same manner as are intact antibodies. Antigen-binding portions/fragments can be produced by recombinant DNA techniques, or by enzymatic or chemical cleavage of intact immunoglobulins.

Unless otherwise indicated, the word “fragment” when used with reference to an antibody, such as in a claim, refers to an antigen binding fragment of the antibody, such that “antibody or fragment” has the same meaning as “antibody or antigen binding fragment thereof.” A “bispecific” or “bifunctional antibody” is an artificial hybrid antibody having two different heavy/light chain pairs, giving rise to two antigen binding sites with specificity for different antigens. Bispecific antibodies can be produced by a variety of methods including fusion of hybridomas or linking of Fab′ fragments. See, e.g., Songsivilai & Lachmann (1990) Clin. Exp. Immunol. 79:315-321; Kostelny et al. (1992) J Immunol. 148, 1547-1553.

The term “monoclonal antibody,” as used herein, refers to an antibody that displays a single binding specificity and affinity for a particular epitope or a composition of antibodies in which all antibodies display a single binding specificity and affinity for a particular epitope. Typically such monoclonal antibodies will be derived from a single cell or nucleic acid encoding the antibody, and will be propagated without intentionally introducing any sequence alterations. Accordingly, the term “human monoclonal antibody” refers to a monoclonal antibody that has variable and optional constant regions derived from human germline immunoglobulin sequences. In one embodiment, human monoclonal antibodies are produced by a hybridoma, for example, obtained by fusing a B cell obtained from a transgenic or transchromosomal non-human animal (e.g., a transgenic mouse having a genome comprising a human heavy chain transgene and a light chain transgene), to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations that occur, for example, during antibody maturation. As known in the art (see, e.g., Lonberg (2005) Nature Biotech. 23(9):1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid sequences that encode the light chain and heavy chain immunoglobulin polypeptides in response to an antigen may not be identical to the original germline sequences, but instead will be substantially identical or similar (i.e., have at least 80% identity).

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. Human antibodies of the present invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies are used synonymously.

A “humanized” antibody refers to an antibody in which some, most or all of the amino acids outside the CDR domains of a non-human antibody, e.g. a mouse antibody, are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody. A “hybrid” antibody refers to an antibody having heavy and light chains of different types, such as a mouse (parental) heavy chain and a humanized light chain, or vice versa.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

“Allotype” refers to naturally occurring variants within a specific isotype group, which variants differ in one or a few amino acids. See, e.g., Jefferis et al. (2009) mAbs 1:1.

The phrases “an antibody recognizing an antigen” and “an antibody specific for an antigen” are used interchangeably herein with the term “an antibody that binds specifically to an antigen.” An “isolated antibody,” as used herein, refers to an antibody that is substantially free of other antibodies having different antigenic specificities (e.g., an isolated antibody that specifically binds to CD40 is substantially free of antibodies that specifically bind antigens other than CD40). An isolated antibody that specifically binds to an epitope of CD40 may, however, have cross-reactivity to other CD40 proteins from different species.

“Effector functions,” deriving from the interaction of an antibody Fc region with certain Fc receptors, include but are not necessarily limited to Clq binding, complement dependent cytotoxicity (CDC), Fc receptor binding, FcγR-mediated effector functions such as ADCC and antibody dependent cell-mediated phagocytosis (ADCP), and down regulation of a cell surface receptor (e.g., the B cell receptor; BCR). Such effector functions generally require the Fc region to be combined with an antigen binding domain (e.g., an antibody variable domain).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIb, or equivalently FcγRIIB) receptor. Various properties of human FcγRs are summarized in Table 1. The majority of innate effector cell types co-express one or more activating FcγR and the inhibitory FcγRIIb, whereas natural killer (NK) cells selectively express one activating Fc receptor (FcγRIII in mice and FcγRIIIA in humans) but not the inhibitory FcγRIIb in mice and humans. Human IgG1 binds to most human Fc receptors and is considered equivalent to murine IgG2a with respect to the types of activating Fc receptors that it binds to.

TABLE 1
Properties of Human FcγRs

| | llelic | ffinity for | Isotype | |
|---|---|---|---|---|
| cγ | variants | human IgG | preference | Cellular distribution |
| cγRI | one | High (K_{D} ~10 nM) | IgG1 = 3 > 4 >> 2 | Monocytes, macrophages, |
| | described | | | activated neutrophils, |
| | | | | dendritic cells? |
| cγRIIA | 131 | Low to | IgG1 > 3 > 2 > 4 | Neutrophils, monocytes, |
| | | medium | | macrophages, eosinophils, |
| | R131 | Low | IgG1 > 3 > 4 > 2 | dendritic cells, platelets |
| cγRIIIA | 158 | Medium | IgG1 = 3 >> 4 > 2 | NK cells, monocytes, |
| | F158 | Low | IgG1 = 3 >> 4 > 2 | macrophages, mast cells, |
| | | | | eosinophils, dendritic cells? |
| cγRIIb | I232 | Low | IgG1 = 3 = 4 > 2 | B cells, monocytes, |
| | T232 | Low | IgG1 = 3 = 4 > 2 | macrophages, dendritic |
| | | | | cells, mast cells |

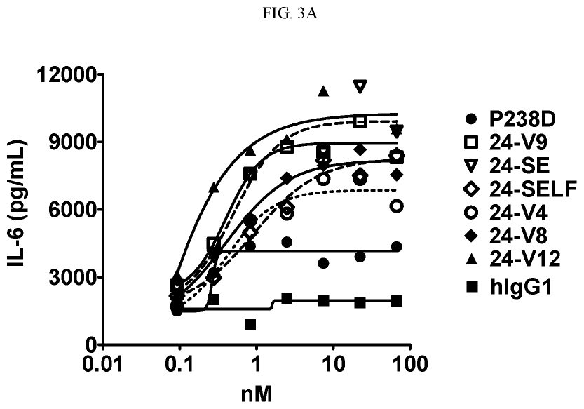

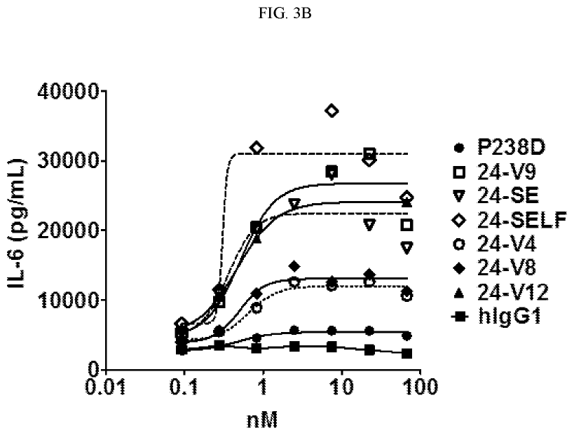

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (Clq) of the classical complement system. Thus, an Fc region comprises the constant region of an antibody excluding the first constant region immunoglobulin domain (e.g., CH1 or CL). In IgG, IgA and IgD antibody isotypes, the Fc region comprises C_{H2} and C_{H3} constant domains in each of the antibody's two heavy chains; IgM and IgE Fc regions comprise three heavy chain constant domains (C_{H} domains 2-4) in each polypeptide chain. For IgG, the Fc region comprises immunoglobulin domains Cγ2 and Cγ3 and the hinge between Cγ1 and Cγ2. Although the boundaries of the Fc region of an immunoglobulin heavy chain might vary, the human IgG heavy chain Fc region is usually defined to stretch from an amino acid residue at position C226 or P230 (or an amino acid between these two amino acids) to the carboxy-terminus of the heavy chain, wherein the numbering is according to the EU index as in Kabat. Kabat et al. (1991) Sequences of Proteins of Immunological Interest, National Institutes of Health, Bethesda, MD; see also FIGS. 3c-3f of U.S. Pat. App. Pub. No. 2008/0248028. The C_{H2} domain of a human IgG Fc region extends from about amino acid 231 to about amino acid 340, whereas the C_{H3} domain is positioned on C-terminal side of a C_{H2} domain in an Fc region, i.e., it extends from about amino acid 341 to about amino acid 447 of an IgG (including a C-terminal lysine). As used herein, the Fc region may be a native sequence Fc, including any allotypic variant, or a variant Fc (e.g., a non-naturally occurring Fc). Fc may also refer to this region in isolation or in the context of an Fc-comprising protein polypeptide such as a “binding protein comprising an Fc region,” also referred to as an “Fc fusion protein” (e.g., an antibody or immunoadhesin).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc include the various allotypes of Fcs. See, e.g., Jefferis et al. (2009) mAbs 1:1.

The term “epitope” or “antigenic determinant” refers to a site on an antigen (e.g., huCD40) to which an immunoglobulin or antibody specifically binds. Epitopes within protein antigens can be formed both from contiguous amino acids (usually a linear epitope) or noncontiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation.

The term “epitope mapping” refers to the process of identification of the molecular determinants on the antigen involved in antibody-antigen recognition. Methods for determining what epitopes are bound by a given antibody are well known in the art and include, for example, immunoblotting and immunoprecipitation assays, wherein overlapping or contiguous peptides from (e.g., from CD40) are tested for reactivity with a given antibody (e.g., anti-CD40 antibody); x-ray crystallography; 2-dimensional nuclear magnetic resonance; yeast display (see Example 6); and HDX-MS (see, e.g., Epitope Mapping Protocols in Methods in Molecular Biology, Vol. 66, G. E. Morris, Ed. (1996)) (see Example 5).

The term “binds to the same epitope” with reference to two or more antibodies means that the antibodies bind to the same segment of amino acid residues, as determined by a given method. Techniques for determining whether antibodies bind to the “same epitope on CD40” with the antibodies described herein include, for example, epitope mapping methods, such as, x-ray analyses of crystals of antigen:antibody complexes, which provides atomic resolution of the epitope, and hydrogen/deuterium exchange mass spectrometry (HDX-MS). Other methods monitor the binding of the antibody to antigen fragments (e.g. proteolytic fragments) or to mutated variations of the antigen where loss of binding due to a modification of an amino acid residue within the antigen sequence is often considered an indication of an epitope component, such as alanine scanning mutagenesis (Cunningham & Wells (1985) Science 244:1081) or yeast display of mutant target sequence variants (see Example 6). In addition, computational combinatorial methods for epitope mapping can also be used. These methods rely on the ability of the antibody of interest to affinity isolate specific short peptides from combinatorial phage display peptide libraries. Antibodies having the same or closely related VH and VL or the same CDR sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competition assays can be conducted as described, for example, in Ed Harlow and David Lane, Cold Spring Harb. Protoc.; 2006; doi:10.1101/pdb.prot4277 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope or to adjacent epitopes (e.g., as evidenced by steric hindrance).

Other competitive binding assays include: solid phase direct or indirect radioimmunoassay (RIA), solid phase direct or indirect enzyme immunoassay (EIA), sandwich competition assay (see Stahli et al. (1983)Methods in Enzymology 9:242); solid phase direct biotin-avidin EIA (see Kirkland et al. (1986) J. Immunol. 137:3614); solid phase direct labeled assay, solid phase direct labeled sandwich assay (see Harlow and Lane (1988) Antibodies: A Laboratory Manual, Cold Spring Harbor Press); solid phase direct label RIA using I-125 label (see Morel et al. (1988) Mol. Immunol. 25(1):7); solid phase direct biotin-avidin EIA (Cheung et al. (1990) Virology 176:546); and direct labeled RIA (Moldenhauer et al. (1990) Scand. J. Immunol. 32:77).

As used herein, the terms “specific binding,” “selective binding,” “selectively binds,” and “specifically binds,” refer to antibody binding to an epitope on a predetermined antigen but not to other antigens. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 surface plasmon resonance instrument using the predetermined antigen, e.g., recombinant human CD40, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, an antibody that “specifically binds to human CD40” refers to an antibody that binds to soluble or cell bound human CD40 with a K_{D} of 10⁻⁷ M or less, such as approximately less than 10⁻⁸ M, 10⁻⁹ M or 10⁻¹⁰ M or even lower. An antibody that “cross-reacts with cynomolgus CD40” refers to an antibody that binds to cynomolgus CD40 with a K_{D} of 10⁻⁷ M or less, such as approximately less than 10⁻⁸ M, 10⁻⁹ M or 10⁻¹⁰ M or even lower.

The term “k_{assoc}” or “kₐ”, as used herein, refers to the association rate constant of a particular antibody-antigen interaction, whereas the term “k_{dis}” or “k_{d},” as used herein, refers to the dissociation rate constant of a particular antibody-antigen interaction. The term “K_{D}”, as used herein, refers to the equilibrium dissociation constant, which is obtained from the ratio of k_{d} to kₐ (i.e., k_{d}/kₐ) and is expressed as a molar concentration (M). K_{D} values for antibodies can be determined using methods well established in the art. A preferred method for determining the K_{D} of an antibody is biolayer interferometry (BLI) analysis, preferably using a ForteBio Octet RED device (see Example 3), surface plasmon resonance, preferably using a biosensor system such as a BIACORE® surface plasmon resonance system (see Example 4), or flow cytometry and Scatchard analysis. The term “EC50” in the context of an in vitro or in vivo assay using an antibody or antigen binding fragment thereof, refers to the concentration of an antibody or an antigen-binding fragment thereof that induces a response that is 50% of the maximal response, i.e., halfway between the maximal response and the baseline.

The term “binds to immobilized CD40” refers to the ability of an antibody described herein to bind to CD40, for example, expressed on the surface of a cell or attached to a solid support.

The term “cross-reacts,” as used herein, refers to the ability of an antibody described herein to bind to CD40 from a different species. For example, an antibody described herein that binds human CD40 may also bind CD40 from another species (e.g., cynomolgus CD40). As used herein, cross-reactivity may be measured by detecting a specific reactivity with purified antigen in binding assays (e.g., SPR, ELISA) or binding to, or otherwise functionally interacting with, cells physiologically expressing CD40. Methods for determining cross-reactivity include standard binding assays as described herein, for example, by BIACORE® surface plasmon resonance (SPR) analysis using a BIACORE® 2000 SPR instrument (Biacore AB, Uppsala, Sweden), or flow cytometric techniques.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein may contain a modification such as, but not limited to, glycosylation, phosphorylation or a disulfide bond. A “protein” may comprise one or more polypeptides.

The term “nucleic acid molecule,” as used herein, is intended to include DNA molecules and RNA molecules. A nucleic acid molecule may be single-stranded or double-stranded, and may be cDNA.

Also provided are “conservative sequence modifications” to the antibody sequence provided herein, i.e. nucleotide and amino acid sequence modifications that do not abrogate the binding of the antibody encoded by the nucleotide sequence or containing the amino acid sequence, to the antigen. For example, modifications can be introduced by standard techniques known in the art, such as site-directed mutagenesis and PCR-mediated mutagenesis. Conservative sequence modifications include conservative amino acid substitutions, in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-CD40 antibody is preferably replaced with another amino acid residue from the same side chain family. Methods of identifying nucleotide and amino acid conservative substitutions that do not eliminate antigen binding are well known in the art. See, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997).

Alternatively, in another embodiment, mutations can be introduced randomly along all or part of an anti-CD40 antibody coding sequence, such as by saturation mutagenesis, and the resulting modified anti-CD40 antibodies can be screened for improved binding activity.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand. For polypeptides, the term “substantial homology” indicates that two polypeptides, or designated sequences thereof, when optimally aligned and compared, are identical, with appropriate amino acid insertions or deletions, in at least about 80% of the amino acids, usually at least about 90% to 95%, and more preferably at least about 98% to 99.5% of the amino acids.

The percent identity between two sequences is a function of the number of identical positions shared by the sequences when the sequences are optimally aligned (i.e., % homology=#of identical positions/total #of positions×100), with optimal alignment determined taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package, using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul et al. (1990) J Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See Ausubel et al., ed. Current Protocols in Molecular Biology, Greene Publishing and Wiley Interscience, New York (1987).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply “expression vectors”). In general, expression vectors of utility in recombinant DNA techniques are often in the form of plasmids. In the present specification, “plasmid” and “vector” may be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and may be a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

An “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition or depletion of a T_{reg} cell. “T effector” (“T_{eff}”) cells refers to T cells (e.g., CD4⁺ and CD8⁺ T cells) with cytolytic activities as well as T helper (Th) cells, which secrete cytokines and activate and direct other immune cells, but does not include regulatory T cells (T_{reg} cells).

As used herein, the term “T cell-mediated response” refers to a response mediated by T cells, including effector T cells (e.g., CD8⁺ cells) and helper T cells (e.g., CD4⁺ cells). T cell mediated responses include, for example, T cell cytotoxicity and proliferation.

As used herein, the term “cytotoxic T lymphocyte (CTL) response” refers to an immune response induced by cytotoxic T cells. CTL responses are mediated primarily by CD8⁺ T cells.

An “immunomodulator” or “immunoregulator” refers to an agent, e.g., a component of a signaling pathway that may be involved in modulating, regulating, or modifying an immune response. “Modulating,” “regulating,” or “modifying” an immune response refers to any alteration in a cell of the immune system or in the activity of such cell (e.g., an effector T cell). Such modulation includes stimulation or suppression of the immune system which may be manifested by an increase or decrease in the number of various cell types, an increase or decrease in the activity of these cells, or any other changes which can occur within the immune system. Both inhibitory and stimulatory immunomodulators have been identified, some of which may have enhanced function in a tumor microenvironment. In preferred embodiments, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying an immune response.

“Immunostimulating therapy” or “immunostimulatory therapy” refers to a therapy that results in increasing (inducing or enhancing) an immune response in a subject for, e.g., treating cancer.

“Potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production. As used herein, “administering” refers to the physical introduction of a composition comprising a therapeutic agent to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, the terms “inhibits” or “blocks” are used interchangeably and encompass both partial and complete inhibition/blocking by at least about 50%, for example, at least about 60%, 70%, 80%, 90%, 95%, 99%, or 100%.

As used herein, “cancer” refers a broad group of diseases characterized by the uncontrolled growth of abnormal cells in the body. Unregulated cell division may result in the formation of malignant tumors or cells that invade neighboring tissues and may metastasize to distant parts of the body through the lymphatic system or bloodstream. The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease. Prophylaxis refers to administration to a subject who does not have a disease, to prevent the disease from occurring or minimize its effects if it does.

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent is a drug that slows cancer progression or promotes cancer regression in a subject. In preferred embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, a prevention of impairment or disability due to the disease affliction, or otherwise amelioration of disease symptoms in the patient. Pharmacological effectiveness refers to the ability of the drug to promote cancer regression in the patient. Physiological safety refers to an acceptably low level of toxicity, or other adverse physiological effects at the cellular, organ and/or organism level (adverse effects) resulting from administration of the drug.

By way of example for the treatment of tumors, a therapeutically effective amount or dosage of the drug preferably inhibits cell growth or tumor growth by at least about 20%, more preferably by at least about 40%, even more preferably by at least about 60%, and still more preferably by at least about 80% relative to untreated subjects. In the most preferred embodiments, a therapeutically effective amount or dosage of the drug completely inhibits cell growth or tumor growth, i.e., preferably inhibits cell growth or tumor growth by 100%. The ability of a compound to inhibit tumor growth can be evaluated using the assays described infra. Inhibition of tumor growth may not be immediate after treatment, and may only occur after a period of time or after repeated administration. Alternatively, this property of a composition can be evaluated by examining the ability of the compound to inhibit cell growth, such inhibition can be measured in vitro by assays known to the skilled practitioner. In other preferred embodiments described herein, tumor regression may be observed and may continue for a period of at least about 20 days, more preferably at least about 40 days, or even more preferably at least about 60 days.

“Combination” therapy, as used herein, unless otherwise clear from the context, is meant to encompass administration of two or more therapeutic agents in a coordinated fashion, and includes, but is not limited to, concurrent dosing. Specifically, combination therapy encompasses both co-administration (e.g. administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. (2011) Blood 117:2423.

The terms “patient” and “subject” refer to any human that receives either prophylactic or therapeutic treatment. For example, the methods and compositions described herein can be used to treat a subject having cancer.

Various aspects described herein are described in further detail in the following subsections.

### I. Anti-CD40 Antibodies

The present application discloses agonistic anti-huCD40 antibodies having desirable properties for use as therapeutic agents in treating diseases such as cancers. These properties include one or more of the ability to bind to human CD40 with high affinity, acceptably low immunogenicity in human subjects, the ability to bind preferentially to FcγRIIb, and the absence of sequence liabilities that might reduce the chemical stability of the antibody.

The anti-CD40 antibodies disclosed herein by sequence bind to specific epitopes on human CD40, as may be determined as described in Examples 5 and 6. Other antibodies that bind to the same or closely related epitopes would likely share these desirable properties, and may be discovered doing competition experiments.

Anti-huCD40 Antibodies that Compete with Anti-huCD40 Antibodies Disclosed Herein

Anti-huCD40 antibodies that compete with the antibodies of the present invention for binding to huCD40 may be raised using immunization protocols similar to those described herein (Examples 1 and 2). Antibodies that compete for binding with the anti-huCD40 antibodies disclosed herein by sequence may also be generated by immunizing mice or other non-human animal with human CD40 or a construct comprising the extracellular domain thereof (residues 21-193 of SEQ ID NO: 1), or by immunizing with a fragment of human CD40 containing the epitope bound by the anti-huCD40 antibodies disclosed herein. The resulting antibodies can be screened for the ability to block binding of 12D6, 5F11, 8E8, 5G7 and/or 19G3 to human CD40 by methods well known in the art, for example blocking binding to fusion protein of the extracellular domain of CD40 and an immunoglobulin Fc domain in a ELISA, or blocking the ability to bind to cells expressing huCD40 on their surface, e.g. by FACS. In various embodiments, the test antibody is contacted with the CD40-Fc fusion protein (or to cells expressing huCD40 on their surface) prior to, at the same time as, or after the addition of 12D6, 5F11, 8E8, 5G7 or 19G3. For example, “binning” experiments may be performed (Example 4) to determine whether a test antibody falls into the same “bin” as an antibodies disclosed herein by sequence, with antibodies disclosed herein by sequence as the “reference” antibodies and the antibodies to be tested as the “test” antibodies. Antibodies that reduce binding of the antibodies disclosed herein by sequence to human CD40 (either as an Fc fusion or on a cell), particularly at roughly stoichiometric concentrations, are likely to bind at the same, overlapping, or adjacent epitopes, and thus may share the desirable functional properties of 12D6, 5F11, 8E8, 5G7 or 19G3.

Accordingly, provided herein are anti-huCD40 antibodies that inhibit the binding of an anti-huCD40 antibodies described herein to huCD40 on cells by at least 10%, 20%, 30%, 40%, 50%, 55%, 60%, 65%, 70%, 75%, 80%, 85%, 90%, 91%, 92%, 93%, 94%, 95%, 96%, 97%, 98%, 99%, or by 100%, and/or whose binding to huCD40 on cells is inhibited by an anti-huCD40 antibodies described herein by at least 10%, 20%, 30%, 40%, 50%, 55%, 60%, 65%, 70%, 75%, 80%, 85%, 90%, 91%, 92%, 93%, 94%, 95%, 96%, 97%, 98%, 99%, or by 100%, e.g., as measured by ELISA or FACS, such as by using the assay described in the following paragraph.

An exemplary competition experiment to determine whether a test antibody blocks the binding of (i.e., “competes with”) a reference antibody, may be conducted as follows: cells expressing CD40 are seeded at 10⁵ cells per sample well in a 96 well plate. The plate is set on ice followed by the addition of unconjugated test antibody at concentrations ranging from 0 to 50 μg/mL (three-fold titration starting from a highest concentration of 50 μg/mL). An unrelated IgG may be used as an isotype control for the first antibody and added at the same concentrations (three-fold titration starting from a highest concentration of 50 μg/mL). A sample pre-incubated with 50 μg/mL unlabeled reference antibody may be included as a positive control for complete blocking (100% inhibition) and a sample without antibody in the primary incubation may be used as a negative control (no competition; 0% inhibition). After 30 minutes of incubation, labeled, e.g., biotinylated, reference antibody is added at a concentration of 2 μg/mL per well without washing. Samples are incubated for another 30 minutes on ice. Unbound antibodies are removed by washing the cells with FACS buffer. Cell-bound labeled reference antibody is detected with an agent that detects the label, e.g., PE conjugated streptavidin (Invitrogen, catalog #521388) for detecting biotin. The samples are acquired on a FACS Calibur Flow Cytometer (BD, San Jose) and analyzed with Flowjo software (Tree Star, Inc, Ashland, OR). The results may be represented as the % inhibition.

Typically, the same experiment is then conducted in the reverse, i.e., the test antibody is the reference antibody and the reference antibody is the test antibody. In certain embodiments, an antibody at least partially (e.g., at least 10%, 20%, 30%, 40%, 50%, 60%, 70%, 80%, or 90%) or completely (100%) blocks the binding of the other antibody to the target, e.g. human CD40 or fragment thereof, and regardless of whether inhibition occurs when one or the other antibody is the reference antibody. A reference antibody and a test antibody “cross-block” binding of each other to the target when the antibodies compete with each other both ways, i.e., in competition experiments in which the reference antibody is added first and in competition experiments in which the test antibody is added first.

Anti-huCD40 antibodies are considered to compete with the anti-huCD40 antibodies disclosed herein if they inhibit binding of 12D6, 5F11, 8E8, 5G7 and/or 19G3 to human CD40 by at least 10%, 20%, 30%, 40%, 50%, 60%, 70%, 80%, 90%, or by 100%, when present at roughly equal concentrations, for example in competition experiments like those described in Example 4. Unless otherwise indicated, an antibody will be considered to compete with an antibody selected from the group consisting of the anti-CD40 antibodies of the present invention if it reduces binding of the selected antibody to human CD40 (SEQ ID NO: 1) by at least 20% when used at a roughly equal molar concentration with the selected antibody, as measured in competition ELISA experiments as outlined in the preceding two paragraphs.

Anti-huCD40 Antibodies that Bind to the Same Epitope

Anti-huCD40 antibodies that bind to the same or similar epitopes to the antibodies disclosed herein may be raised using immunization protocols similar to those described herein (Examples 1 and 2). The resulting antibodies can be screened for high affinity binding to human CD40 (Example 3). Selected antibodies can then be studied in yeast display assay in which sequence variants of huCD40 are presented on the surface of yeast cells (Example 6), or by hydrogen-deuterium exchange experiments (Example 5), to determine the precise epitope bound by the antibody.

Epitope determinations may be made by any method known in the art. In various embodiments, anti-huCD40 antibodies are considered to bind to the same epitope as an anti-huCD40 mAb disclosed herein if they make contact with one or more of the same residues within at least one region of huCD40; if they make contacts with a majority of the residues within at least one region of huCD40; if they make contacts with a majority of the residues within each region of huCD40; if they make contact with a majority of contacts along the entire length of huCD40; if they make contacts within all of the same distinct regions of human CD40; if they make contact with all of the residues at any one region on human CD40; or if they make contact with all of the same residues at all of the same regions. Epitope “regions” are clusters of residues along the primary sequence.

Techniques for determining antibodies that bind to the “same epitope on huCD40” with the antibodies described herein include x-ray analyses of crystals of antigen:antibody complexes, which provides atomic resolution of the epitope. Other methods monitor the binding of the antibody to antigen fragments or mutated variations of the antigen where loss of binding due to a modification of an amino acid residue within the antigen sequence is often considered an indication of an epitope component. Methods may also rely on the ability of an antibody of interest to affinity isolate specific short peptides (either in native three dimensional form or in denatured form) from combinatorial phage display peptide libraries or from a protease digest of the target protein. The peptides are then regarded as leads for the definition of the epitope corresponding to the antibody used to screen the peptide library. For epitope mapping, computational algorithms have also been developed that have been shown to map conformational discontinuous epitopes.

The epitope or region comprising the epitope can also be identified by screening for binding to a series of overlapping peptides spanning CD40. Alternatively, the method of Jespers et al. (1994) Biotechnology 12:899 may be used to guide the selection of antibodies having the same epitope and therefore similar properties to the an anti-CD40 antibodies described herein. Using phage display, first the heavy chain of the anti-CD40 antibody is paired with a repertoire of (preferably human) light chains to select a CD40-binding antibody, and then the new light chain is paired with a repertoire of (preferably human) heavy chains to select a (preferably human) CD40-binding antibody having the same epitope or epitope region as an anti-huCD40 antibody described herein. Alternatively variants of an antibody described herein can be obtained by mutagenesis of cDNA encoding the heavy and light chains of the antibody.

Alanine scanning mutagenesis, as described by Cunningham & Wells (1989) Science 244: 1081, or some other form of point mutagenesis of amino acid residues in CD40 (such as the yeast display method provided at Example 6) may also be used to determine the functional epitope for an anti-CD40 antibody.

The epitope or epitope region (an “epitope region” is a region comprising the epitope or overlapping with the epitope) bound by a specific antibody may also be determined by assessing binding of the antibody to peptides comprising fragments of CD40. A series of overlapping peptides encompassing the sequence of CD40 (e.g., human CD40) may be synthesized and screened for binding, e.g. in a direct ELISA, a competitive ELISA (where the peptide is assessed for its ability to prevent binding of an antibody to CD40 bound to a well of a microtiter plate), or on a chip. Such peptide screening methods may not be capable of detecting some discontinuous functional epitopes, i.e. functional epitopes that involve amino acid residues that are not contiguous along the primary sequence of the CD40 polypeptide chain.

An epitope may also be identified by MS-based protein footprinting, such as hydrogen/deuterium exchange mass spectrometry (HDX-MS) and Fast Photochemical Oxidation of Proteins (FPOP). HDX-MS may be conducted, e.g., as further described at Wei et al. (2014) Drug Discovery Today 19:95, the methods of which are specifically incorporated by reference herein. See also Example 5. FPOP may be conducted as described, e.g., in Hambley & Gross (2005) J. American Soc. Mass Spectrometry 16:2057, the methods of which are specifically incorporated by reference herein.

The epitope bound by anti-CD40 antibodies may also be determined by structural methods, such as X-ray crystal structure determination (e.g., WO 2005/044853), molecular modeling and nuclear magnetic resonance (NMR) spectroscopy, including NMR determination of the H-D exchange rates of labile amide hydrogens in CD40 when free and when bound in a complex with an antibody of interest (Zinn-Justin et al. (1992) Biochemistry 31:11335; Zinn-Justin et al. (1993) Biochemistry 32:6884).

With regard to X-ray crystallography, crystallization may be accomplished using any of the known methods in the art (e.g. Giege et al. (1994) Acta Crystallogr. D50:339; McPherson (1990) Eur. J Biochem. 189:1), including microbatch (e.g. Chayen (1997) Structure 5:1269), hanging-drop vapor diffusion (e.g. McPherson (1976) J Biol. Chem. 251:6300), seeding and dialysis. It is desirable to use a protein preparation having a concentration of at least about 1 mg/mL and preferably about 10 mg/mL to about 20 mg/mL. Crystallization may be best achieved in a precipitant solution containing polyethylene glycol 1000-20,000 (PEG; average molecular weight ranging from about 1000 to about 20,000 Da), preferably about 5000 to about 7000 Da, more preferably about 6000 Da, with concentrations ranging from about 10% to about 30% (w/v). It may also be desirable to include a protein stabilizing agent, e.g. glycerol at a concentration ranging from about 0.5% to about 20%. A suitable salt, such as sodium chloride, lithium chloride or sodium citrate may also be desirable in the precipitant solution, preferably in a concentration ranging from about 1 mM to about 1000 mM. The precipitant is preferably buffered to a pH of from about 3.0 to about 5.0, preferably about 4.0. Specific buffers useful in the precipitant solution may vary and are well known in the art (Scopes, Protein Purification: Principles and Practice, Third ed., (1994) Springer-Verlag, New York). Examples of useful buffers include, but are not limited to, HEPES, Tris, MES and acetate. Crystals may be grow at a wide range of temperatures, including 2° C., 4° C., 8° C. and 26° C.

Antibody:antigen crystals may be studied using well-known X-ray diffraction techniques and may be refined using computer software such as X-PLOR (Yale University, 1992, distributed by Molecular Simulations, Inc.; see e.g. Blundell & Johnson (1985)Meth. Enzymol. 114 & 115, H. W. Wyckoff et al., eds., Academic Press; U.S. Patent Application Publication No. 2004/0014194), and BUSTER (Bricogne (1993) Acta Cryst. D49:37-60; Bricogne (1997)Meth. Enzymol. 276A:361-423, Carter & Sweet, eds.; Roversi et al. (2000) Acta Cryst. D56:1313-1323), the disclosures of which are hereby incorporated by reference in their entireties.

Unless otherwise indicated, and with reference to the claims, the epitope bound by an antibody is the epitope as determined by HDX-MS methods, substantially as described in Example 5.

Anti-CD40 Antibodies that Bind with High Affinity

In some embodiments the anti-huCD40 antibodies of the present invention bind to huCD40 with high affinity, like the anti-huCD40 antibodies disclosed herein, increasing their likelihood of being effective therapeutic agents. In various embodiments anti-huCD40 antibodies of the present invention bind to huCD40 with a K_{D} of less than 10 nM, 5 nM, 2 nM, 1 nM, 300 μM or 100 μM. In other embodiments, the anti-huCD40 antibodies of the present invention bind to huCD40 with a K_{D} between 2 nM and 100 μM. Standard assays to evaluate the binding ability of the antibodies toward huCD40 include ELISAs, RIAs, Western blots, biolayer interferometry (BLI) (see Example 3) and BIACORE® SPR analysis (see Example 4).

### Anti-CD40 Antibody Sequence Variants

Some variability in the antibody sequences disclosed herein may be tolerated and still maintain the desirable properties of the antibody. The CDR regions are delineated using the Kabat system (Kabat, E. A., et al. (1991) Sequences of Proteins of Immunological Interest, Fifth Edition, U.S. Department of Health and Human Services, NIH Publication No. 91-3242). Accordingly, the present invention further provides anti-huCD40 antibodies comprising CDR sequences that are at least 70%, 75%, 80%, 85%, 90%, 95%, 96%, 97%, 98%, or 99% identical to the CDR sequences of the antibodies disclosed herein (i.e. 12D6, 5F11, 8E8, 5G7 and 19G3 and humanized derivatives thereof). The present invention also provides anti-huCD40 antibodies comprising heavy and/or light chain variable domain sequences that are at least 70%, 75%, 80%, 85%, 90%, 95%, 96%, 97%, 98%, or 99% identical to the heavy and/or light chain variable domain sequences of the antibodies disclosed herein (i.e. 12D6, 5F11, 8E8, 5G7 and 19G3 and humanized derivatives thereof).

Anti-CD40 Antibodies Sharing CDR Sequences or Derived from the Same Murine Germlines

Given that antigen-binding specificity is determined primarily by the CDRs, antibodies sharing CDRs sequences with antibodies disclosed herein (i.e. 12D6, 5F11, 8E8, 5G7 and 19G3) are likely to share their desirable properties. In some embodiments, anti-huCD40 antibodies of the present invention comprises heavy and light chain variable regions derived from the same murine V region and J region germline sequences as antibody 12D6, 5F11, 8E8, 5G7 or 19G3. Antibody 12D6 has a heavy chain derived from murine germlines VH1-39_01 and IGHJ4, and light chain germlines VK1-110_01 and IGKJ1. Antibody 5F11 has a heavy chain derived from murine germlines VH1-4_02 and IGHJ3, and light chain germlines VK3-5_01 and IGKJ5. Antibody 8E8 has a heavy chain derived from murine germlines VH1-80_01 and IGHJ2, and light chain germlines VK1-110_01 and IGKJ2. Antibody 5G7 has a heavy chain derived from murine germlines VH1-18_01 and IGHJ4, and light chain germlines VK10⁻⁹⁶_01 and IGKJ2. Antibody 19G3 has a heavy chain derived from murine germlines VH5-9-4_01 and IGHJ3, and light chain germlines VK1-117_01 and IGKJ2. Heavy chain D region germline sequences (making up part of CDRH3) are not specified, as they are often difficult to assign given their high variability, and thus antibodies of the present invention may comprise heavy chains derived from the listed V and J region germlines and any D region germline. Other antibodies that bind to human CD40 and are derived from some or all of these germline sequences are likely to be closely related in sequence, particularly those derived from the same V-region genes, and thus would be expected to share the same desirable properties.

As used herein, a murine antibody comprises heavy or light chain variable regions that are “derived from” a particular germline sequence if the variable regions of the antibody are obtained from a system that uses murine germline immunoglobulin genes, and the antibody sequence is sufficiently related to the germline that it is more likely derived from the given germline than from any other. Such systems include immunizing a mouse with the antigen of interest. The murine germline immunoglobulin sequence(s) from which the sequence of an antibody is “derived” can be identified by comparing the amino acid sequence of the antibody to the amino acid sequences of murine germline immunoglobulins and selecting the germline immunoglobulin sequence that is closest in sequence (i.e., greatest % identity) to the sequence of the antibody. A murine antibody that is “derived from” a particular germline immunoglobulin sequence may contain amino acid differences as compared to the germline sequence due to, for example, naturally-occurring somatic mutations or intentional introduction of site-directed mutation. However, a selected murine antibody typically is at least 90% identical in amino acids sequence to an amino acid sequence encoded by a germline immunoglobulin gene (e.g. V regions). In certain cases, a murine antibody may be at least 95%, or even at least 96%, 97%, 98%, or 99% identical in amino acid sequence to the amino acid sequence encoded by the germline immunoglobulin gene (e.g. V regions). Typically, an antibody derived from a particular murine germline sequence will display no more than 10 amino acid differences from the amino acid sequence encoded by the germline immunoglobulin gene (e.g. V regions). In certain cases, the murine antibody may comprise no more than 5, or even no more than 4, 3, 2, or 1 amino acid difference from the amino acid sequence encoded by the germline immunoglobulin gene (e.g. V regions).

### II. Engineered and Modified Antibodies

VH and VL regions

Also provided are engineered and modified antibodies that can be prepared using an antibody having one or more of the V_{H} and/or V_{L} sequences disclosed herein as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Such grafting is of particular use in humanizing non-human anti-CD40 antibodies that compete for binding with the anti-huCD40 antibodies disclosed herein and/or bind to the same epitope as the anti-huCD40 antibodies disclosed herein. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific reference antibodies by constructing expression vectors that include CDR sequences from the specific reference antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. Sci. USA 86:10029-10033; U.S. Pat. No. 5,225,539 to Winter, and U.S. Pat. Nos. 5,530,101; 5,585,089; 5,693,762 and 6,180,370 to Queen et al.)

Such framework sequences can be obtained from public DNA databases or published references that include germline antibody gene sequences. For example, germline DNA sequences for human heavy and light chain variable region genes can be found in the “VBase” human germline sequence database, as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J Mol. Biol. 227:776-798; and Cox, J. P. L. et al. (1994) “A Directory of Human Germ-line V_{H} Segments Reveals a Strong Bias in their Usage” Eur. J Immunol. 24:827-836; the contents of each of which are expressly incorporated herein by reference.

Preferred framework sequences for use in the antibodies described herein are those that are structurally similar to the framework sequences used by antibodies described herein. The V_{H} CDR1, 2 and 3 sequences, and the V_{L} CDR1, 2 and 3 sequences, can be grafted onto framework regions that have the identical sequence as that found in the germline immunoglobulin gene from which the framework sequence derive, or the CDR sequences can be grafted onto framework regions that contain up to 20, preferably conservative, amino acid substitutions as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Engineered antibodies described herein include those in which modifications have been made to framework residues within V_{H} and/or V_{L}, e.g. to improve the properties of the antibody. Often such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 2003/0153043 by Carr et al.

Another type of variable region modification is to mutate amino acid residues within the CDR regions to improve one or more binding properties (e.g., affinity) of the antibody of interest. Site-directed mutagenesis or PCR-mediated mutagenesis can be performed to introduce the mutation(s) and the effect on antibody binding, or other functional property of interest. Preferably conservative modifications are introduced. The mutations may be amino acid additions, deletions, or preferably substitutions. Moreover, typically no more than one, two, three, four or five residues within a CDR region are altered.

Methionine residues in CDRs of antibodies can be oxidized, resulting in potential chemical degradation and consequent reduction in potency of the antibody. Accordingly, also provided are anti-CD40 antibodies that have one or more methionine residues in the heavy and/or light chain CDRs replaced with amino acid residues that do not undergo oxidative degradation. Similarly, deamidation sites may be removed from anti-CD40 antibodies, particularly in the CDRs. Potential glycosylation sites within the antigen binding domain are preferably eliminated to prevent glycosylation that may interfere with antigen binding. See, e.g., U.S. Pat. No. 5,714,350.

### Targeted Antigen Binding

In various embodiments, the antibody of the present invention is modified to selectively block antigen binding in tissues and environments where antigen binding would be detrimental, but allow antigen binding where it would be beneficial. In one embodiment, a blocking peptide “mask” is generated that specifically binds to the antigen binding surface of the antibody and interferes with antigen binding, which mask is linked to each of the binding arms of the antibody by a peptidase cleavable linker. See, e.g., U.S. Pat. No. 8,518,404 to CytomX. Such constructs are useful for treatment of cancers in which protease levels are greatly increased in the tumor microenvironment compared with non-tumor tissues. Selective cleavage of the cleavable linker in the tumor microenvironment allows disassociation of the masking/blocking peptide, enabling antigen binding selectively in the tumor, rather than in peripheral tissues in which antigen binding might cause unwanted side effects.

Alternatively, in a related embodiment, a bivalent binding compound (“masking ligand”) comprising two antigen binding domains is developed that binds to both antigen binding surfaces of the (bivalent) antibody and interfere with antigen binding, in which the two binding domains masks are linked to each other (but not the antibody) by a cleavable linker, for example cleavable by a peptidase. See, e.g., Int'l Pat. App. Pub. No. WO 2010/077643 to Tegopharm Corp. Masking ligands may comprise, or be derived from, the antigen to which the antibody is intended to bind, or may be independently generated. Such masking ligands are useful for treatment of cancers in which protease levels are greatly increased in the tumor microenvironment compared with non-tumor tissues. Selective cleavage of the cleavable linker in the tumor microenvironment allows disassociation of the two binding domains from each other, reducing the avidity for the antigen-binding surfaces of the antibody. The resulting dissociation of the masking ligand from the antibody enables antigen binding selectively in the tumor, rather than in peripheral tissues in which antigen binding might cause unwanted side effects.

### Fcs and Modified Fcs

Antibodies of the present invention may comprise the variable domains of the invention combined with constant domains comprising different Fc regions, selected based on the biological activities (if any) of the antibody for the intended use. Salfeld (2007) Nat. Biotechnol. 25:1369. Human IgGs, for example, can be classified into four subclasses, IgG1, IgG2, IgG3, and IgG4, and each these of these comprises an Fc region having a unique profile for binding to one or more of Fcγ receptors (activating receptors FcγRI (CD64), FcγRIIA, FcγRIIC (CD32a,c); FcγRIIIA and FcγRIIIB (CD16a,b) and inhibiting receptor FcγRIIB (CD32b), and for the first component of complement (Clq). Human IgG1 and IgG3 bind to all Fcγ receptors; IgG2 binds to FcγRIIA_{H131}, and with lower affinity to FcγRIIA_{R131} FcγRIIIAᵥ₁₅₈; IgG4 binds to FcγRI, FcγRIIA, FcγRIIB, FcγRIIC, and FcγRIIIAᵥ₁₅₈; and the inhibitory receptor FcγRIIB has a lower affinity for IgG1, IgG2 and IgG3 than all other Fcγ receptors. Bruhns et al. (2009) Blood 113:3716. Studies have shown that FcγRI does not bind to IgG2, and FcγRIIIB does not bind to IgG2 or IgG4. Id. In general, with regard to ADCC activity, human IgG1≥IgG3>>IgG4≥IgG2. As a consequence, for example, an IgG1 constant domain, rather than an IgG2 or IgG4, might be chosen for use in a drug where ADCC is desired; IgG3 might be chosen if activation of FcγRIIIA-expressing NK cells, monocytes of macrophages; and IgG4 might be chosen if the antibody is to be used to desensitize allergy patients. IgG4 may also be selected if it is desired that the antibody lack all effector function.

Anti-huCD40 variable regions described herein may be linked (e.g., covalently linked or fused) to an Fc, e.g., an IgG1, IgG2, IgG3 or IgG4 Fc, which may be of any allotype or isoallotype, e.g., for IgG1: G1m, G1ml(a), G1m2(x), G1m3(f), G1m17(z); for IgG2: G2m, G2m23(n); for IgG3: G3m, G3m21(g1), G3m28(g5), G3m11(b0), G3m5(b1), G3m13(b3), G3m14(b4), G3m10(b5), G3m15(s), G3m16(t), G3m6(c3), G3m24(c5), G3m26(u), G3m27(v). See, e.g., Jefferis et al. (2009) mAbs 1:1). Selection of allotype may be influenced by the potential immunogenicity concerns, e.g. to minimize the formation of anti-drug antibodies.

In preferred embodiments, anti-CD40 antibodies of the present invention have an Fc that binds to or has enhanced binding to FcγRIIb, which can provide enhanced agonism. See, e.g., WO 2012/087928; Li & Ravetch (2011) Science 333:1030; Wilson et al. (2011) Cancer Cell 19:101; White et al. (2011) J Immunol. 187:1754. Variable regions described herein may be linked to Fc variants that enhance affinity for the inhibitory receptor FcγRIIb, e.g. to enhance apoptosis-inducing or adjuvant activity. Li & Ravetch (2012) Proc. Nat'l Acad. Sci. USA 109:10966; U S. Pat. App. Pub. 2014/0010812. Such variants may provide an antibody with immunomodulatory activities related to FcγRIIb⁺ cells, including for example B cells and monocytes. In one embodiment, the Fc variants provide selectively enhanced affinity to FcγRIIb relative to one or more activating receptors, Such variants may also exhibit enhanced FcR-mediated cross-linking, resulting in enhanced therapeutic efficacy. Modifications for altering binding to FcγRIIb include one or more modifications at a position selected from the group consisting of 234, 235, 236, 237, 239, 266, 267, 268, 325, 326, 327, 328, and 332, according to the EU index. Exemplary substitutions for enhancing FcγRIIb affinity include but are not limited to 234D, 234E, 234F, 234W, 235D, 235F, 235R, 235Y, 236D, 236N, 237D, 237N, 239D, 239E, 266M, 267D, 267E, 268D, 268E. 327D, 327E, 328F, 328W, 328Y, and 332E. Exemplary substitutions include 235Y, 236D, 2391), 266M, 267E, 2681), 268E, 328F, 328W. and 328Y. Other Fc variants for enhancing binding to FcγRIIb include 235Y-267E, 236D-267E, 239D-268D, 239D-267E, 267E-268D, 267E-268E, and 267E-328F. Specifically, the S267E, G236D, S239D, L328F and 1332E variants, including the S267E-L328F double variant, of human IgG1 are of particular value in specifically enhancing affinity for the inhibitory FcγRIIb receptor. Chu et al. (2008)Mol. Immunol. 45:3926; U.S. Pat. App. Pub. 2006/024298; WO 2012/087928. Enhanced specificity for FcγRIIb (as distinguished from FcγRIIa_{R131}) may be obtained by adding the P238D substitution and other mutations (Mimoto et al. (2013) Protein. Eng. Des. & Selection 26:589; WO 2012/1152410), as well as V262E and V264E (Yu et al. (2013) J Am. Chem. Soc. 135:9723, and WO 2014/184545. See Table 4.

### Half-Life Extension

In certain embodiments, the antibody is modified to increase its biological half-life. Various approaches are possible. For example, this may be done by increasing the binding affinity of the Fc region for FcRn. In one embodiment, the antibody is altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary Fc variants that increase binding to FcRn and/or improve pharmacokinetic properties include substitutions at positions 259, 308, and 434, including for example 259I, 308F, 428L, 428M, 434S, 434H, 434F, 434Y, and 434M. Other variants that increase Fc binding to FcRn include: 250E, 250Q, 428L, 428F, 250Q/428L (Hinton et al. (2004)J Biol. Chem. 279(8): 6213-6216, Hinton et at (2006) Journal of Immunology 176:346-356), 256A, 272A, 305A, 307A, 31 1A, 312A, 378Q, 380A, 382A, 434A (Shields et al. (2001). Journal of Biological Chemistry 276(9):6591-6604), 252F, 252Y, 252W, 254T, 256Q, 256E, 256D, 433R, 434F, 434Y 252Y/254T/256E, 433K1434F/436H (Dall'Acqua et al. (2002) Journal of Immunology 169:5171-5180, Dall'Acqua et al. (2006) Journal of Biological Chemistry 281:23514-23524). See U.S. Pat. No. 8,367,805.

Modification of certain conserved residues in IgG Fc (I253, H310, Q311, H433, N434), such as the N434A variant (Yeung et al. (2009). J. Immunol. 182:7663), have been proposed as a way to increase FcRn affinity, thus increasing the half-life of the antibody in circulation, See WO 98/023289. The combination Fc variant comprising M428L and N434S has been shown to increase FcRn binding and increase serum half-life up to five-fold. Zalevsky et al. (2010) Nat. Biotechnol. 28:157. The combination Fe variant comprising T307A, E380A and N434A modifications also extends half-life of I1C3 antibodies. Petkova et al. (2006) Int. Immunol. 18:1759. In addition, combination Fc variants comprising M252Y-M428L, M428L-N4341-1, M428L-N434F, M428L-N434Y, M428L-N434A, M428L-N434M, and M428L-N434S variants have also been shown to extend half-life. See WO 2009/086320.

Further, a combination Fc variant comprising M252Y, S254T and T256E increases half-life-nearly 4-fold. Dall'Acqua et al. (2006) J. Biol. Chem. 281:23514. A related IgG1 modification providing increased FcRn affinity but reduced pH dependence (M252Y-S254T-T256E-11433K-N434F) has been used to create an IgG1 construct (“MST-HN Abdeg”) for use as a competitor to prevent binding of other antibodies to FcRn, resulting in increased clearance of that other antibody, either endogenous IgG (e.g. in an autoimmune setting) or another exogenous (therapeutic) mAb. Vaccaro et at. (2005) Nat. Biotechnol. 23:1283; WO 2006/130834.

Other modifications for increasing FcRn binding are described in Yeung et al. (2010) J. Immunol. 182:7663-7671; 6,277,375; 6,821,505; WO 97/34631; and WO 2002/060919.

In certain embodiments, hybrid IgG isotypes may be used to increase FcRn binding, and potentially increase half-life. For example, an IgG1/IgG3 hybrid variant may be constructed by substituting IgG1 positions in the CH2 and/or CH3 region with the amino acids from IgG3 at positions where the two isotypes differ. Thus a hybrid variant IgG antibody may be constructed that comprises one or more substitutions, e.g., 274Q, 276K, 300F, 339T, 356E, 358M, 384S, 392N, 397M, 4221, 435R, and 436F, In other embodiments described herein, an IgG1/IgG2 hybrid variant may be constructed by substituting IgG2 positions in the (CH2 and/or CH3 region with amino acids from IgG1 at positions where the two isotypes differ. Thus a hybrid variant IgG antibody may be constructed that comprises one or more substitutions, e.g., one or more of the following amino acid substitutions: 233E, 234L, 235L, −236G (referring to an insertion of a gly cine at position 236), and 327A. See U.S. Pat. No. 8,629,113. A hybrid of IgG1/IgG2/IgG4 sequences has been generated that purportedly increases serum half-life and improves expression. U.S. Pat. No. 7,867,491 (sequence number 18 therein).

The serum half-life of the antibodies of the present invention can also be increased by pegylation. An antibody can be pegylated to, for example, increase the biological (e.g., serum) half-life of the antibody. To pegylate an antibody, the antibody, or fragment thereof, typically is reacted with a polyethylene glycol (PEG) reagent, such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies described herein. See for example, EP 0154316 by Nishimura et al. and EP 0401384 by Ishikawa et al.

Alternatively, under some circumstances it may be desirable to decrease the half-life of an antibody of the present invention, rather than increase it. Modifications such as 1253A (Homick et al. (2000) J Nucl. Med. 41:355) and H435A/R, 1253A or H310A (Kim et al. (2000) Eur. J. Immunol. 29:2819) in Fc of human IgG1 can decrease FcRn binding, thus decreasing half-life (increasing clearance) for use in situations where rapid clearance is preferred, such a medical imaging. See also Kenanova et al. (2005) Cancer Res. 65:622. Other means to enhance clearance include formatting the antigen binding domains of the present invention as antibody fragments lacking the ability to bind FcRn, such as Fab fragments. Such modification can reduce the circulating half-life of an antibody from a couple of weeks to a matter of hours. Selective PEGylation of antibody fragments can then be used to fine-tune (increase) the half-life of the antibody fragments if necessary. Chapman et al. (1999) Nat. Biotechnol. 17:780. Antibody fragments may also be fused to human serum albumin, e.g. in a fusion protein construct, to increase half-life. Yeh et al. (1992) Proc. Nat'l Acad. Sci. USA 89:1904. Alternatively, a bispecific antibody may be constructed with a first antigen binding domain of the present invention and a second antigen binding domain that binds to human serum albumin (HSA). See Int'l Pat. Appl. Pub. WO 2009/127691 and patent references cited therein. Alternatively, specialized polypeptide sequences can be added to antibody fragments to increase half-life, e.g. “XTEN” polypeptide sequences. Schellenberger et al. (2009) Nat. Biotechnol. 27:1186; Int'l Pat. Appl. Pub. WO 2010/091122.

### Additional Fc Variants

When using an IgG4 constant domain, it is usually preferable to include the substitution S228P, which mimics the hinge sequence in IgG1 and thereby stabilizes IgG4 molecules, e.g. reducing Fab-arm exchange between the therapeutic antibody and endogenous IgG4 in the patient being treated. Labrijn et al. (2009) Nat. Biotechnol. 27:767; Reddy et al. (2000) J. Immunol. 164:1925.

A potential protease cleavage site in the hinge of IgG1 constructs can be eliminated by D221G and K222S modifications, increasing the stability of the antibody. WO 2014/043344.

The affinities and binding properties of an Fe variant for its ligands (Fe receptors) may be determined by a variety of in vitro assay methods (biochemical or immunological based assays) known in the art including but not limited to, equilibrium methods (e.g., enzyme-linked immunosorbent assay (ELISA), or radioimmunoassay (RIA)), or kinetics (e.g. BIACORE® SPR analysis), and other methods such as indirect binding assays, competitive inhibition assay s, fluorescence resonance energy transfer (FRET), gel electrophoresis and chromatography (e.g., gel filtration). These and other methods may use a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

In still other embodiments, the glycosylation of an antibody is modified to increase or decrease effector function. For example, an aglycoslated antibody can be made that lacks all effector function by mutating the conserved asparagine residue at position 297 (e.g. N297A), thus abolishing complement and FcγRI binding. Bolt et al. (1993) Eur. J. Immunol. 23:403. See also Tao & Morrison (1989) J. Immunol. 143:2595 (using N297Q in IgG1 to eliminate glycosylation at position 297).

Although aglycosylated antibodies generally lack effector function, mutations can be introduced to restore that function. Aglycosylated antibodies, e.g. those resulting from N297A/C/D/or H mutations or produced in systems (e.g. E. coli) that do not glycosylate proteins, can be further mutated to restore FcγR binding, e.g. S298G and/or T299A/G/or H (WO 2009/079242), or E382V and M4281 (Jung et al. (2010) Proc. Nat'l Acad. Sci. USA 107:604).

Glycoengineering can also be used to modify the anti-inflammatory properties of an IgG construct by changing the α2,6 sialyl content of the carbohydrate chains attached at Asn297 of the Fc regions, wherein an increased proportion of α2,6 sialylated forms results in enhanced anti-inflammatory effects. See Nimmerjahn et al. (2008) Ann. Rev. Immunol. 26:513. Conversely, reduction in the proportion of antibodies having α2,6 sialylated carbohydrates may be useful in cases where anti-inflammatory properties are not wanted. Methods of modifying α2,6 sialylation content of antibodies, for example by selective purification of α2,6 sialylated forms or by enzymatic modification, are provided at U.S. Pat. Appl. Pub. No. 2008/0206246. In other embodiments, the amino acid sequence of the Fc region may be modified to mimic the effect of α2,6 sialylation, for example by inclusion of an F241A modification. WO 2013/095966.

### III. Antibody Physical Properties

Antibodies described herein can contain one or more glycosylation sites in either the light or heavy chain variable region. Such glycosylation sites may result in increased immunogenicity of the antibody or an alteration of the pK of the antibody due to altered antigen binding (Marshall et al. (1972) Ann. Rev. Biochem. 41:673-702; Gala and Morrison (2004) J Immunol. 172:5489-94; Wallick et al. (1988) J Exp. Med. 168:1099-109; Spiro (2002) Glycobiology 12:43R-56R; Parekh et al. (1985) Nature 316:452-7; Mimura et al. (2000) Mol Immunol 37:697-706). Glycosylation has been known to occur at motifs containing an N-X-S/T sequence. In some instances, it is preferred to have an anti-huCD40 antibody that does not contain variable region glycosylation. This can be achieved either by selecting antibodies that do not contain the glycosylation motif in the variable region or by mutating residues within the glycosylation region.

In certain embodiments, the antibodies described herein do not contain asparagine isomerism sites. The deamidation of asparagine may occur on N-G or D-G sequences and result in the creation of an isoaspartic acid residue that introduces a kink into the polypeptide chain and decreases its stability (isoaspartic acid effect).

Each antibody will have a unique isoelectric point (pI), which generally falls in the pH range between 6 and 9.5. The pI for an IgG1 antibody typically falls within the pH range of 7-9.5 and the pI for an IgG4 antibody typically falls within the pH range of 6-8. There is speculation that antibodies with a pI outside the normal range may have some unfolding and instability under in vivo conditions. Thus, it is preferred to have an anti-CD40 antibody that contains a pI value that falls in the normal range. This can be achieved either by selecting antibodies with a pI in the normal range or by mutating charged surface residues.

Each antibody will have a characteristic melting temperature, with a higher melting temperature indicating greater overall stability in vivo (Krishnamurthy & Manning (2002) Curr. Pharm. Biotechnol. 3:361-71). Generally, it is preferred that the T_{M1} (the temperature of initial unfolding) be greater than 60° C., preferably greater than 65° C., even more preferably greater than 70° C. The melting point of an antibody can be measured using differential scanning calorimetry (Chen et al. (2003) Pharm Res 20:1952-60; Ghirlando et al. (1999) Immunol Lett. 68:47-52) or circular dichroism (Murray et al. (2002) J Chromatogr. Sci. 40:343-9). In a preferred embodiment, antibodies are selected that do not degrade rapidly. Degradation of an antibody can be measured using capillary electrophoresis (CE) and MALDI-MS (Alexander & Hughes (1995) Anal Chem. 67:3626-32).

In another preferred embodiment, antibodies are selected that have minimal aggregation effects, which can lead to the triggering of an unwanted immune response and/or altered or unfavorable pharmacokinetic properties. Generally, antibodies are acceptable with aggregation of 25% or less, preferably 20% or less, even more preferably 15% or less, even more preferably 10% or less and even more preferably 5% or less. Aggregation can be measured by several techniques, including size-exclusion column (SEC), high performance liquid chromatography (HPLC), and light scattering.

### IV. Nucleic Acid Molecules

Another aspect described herein pertains to nucleic acid molecules that encode the antibodies described herein. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and may or may not contain intronic sequences. In a certain embodiments, the nucleic acid is a cDNA molecule.

Nucleic acids described herein can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (hinge, CH1, CH2 and/or CH3). The sequences of human heavy chain constant region genes are known in the art (see e.g., Kabat, E. A. et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, for example, an IgG1 region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al. (1990) Nature 348:552-554).

### V. Antibody Generation

Various antibodies of the present invention, e.g. those that compete with or bind to the same epitope as the anti-human CD40 antibodies disclosed herein, can be produced using a variety of known techniques, such as the standard somatic cell hybridization technique described by Kohler and Milstein, Nature 256:495 (1975). Although somatic cell hybridization procedures are preferred, in principle, other techniques for producing monoclonal antibodies also can be employed, e.g., viral or oncogenic transformation of B lymphocytes, phage display technique using libraries of human antibody genes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies described herein can be prepared based on the sequence of a murine monoclonal antibody prepared as described above. DNA encoding the heavy and light chain immunoglobulins can be obtained from the murine hybridoma of interest and engineered to contain non-murine (e.g. human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In one embodiment, the antibodies described herein are human monoclonal antibodies. Such human monoclonal antibodies directed against human CD40 can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb mouse® (Medarex, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368(6474):856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N. Y. Acad. Sci. 764:536-546). The preparation and use of HuMab mice, and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6: 579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In certain embodiments, antibodies described herein are raised using a mouse that carries human immunoglobulin sequences on transgenes and transchromosomes, such as a mouse that carries a human heavy chain transgene and a human light chain transchromosome. Such mice, referred to herein as “KM mice”, are described in detail in PCT Publication WO 02/43478 to Ishida et al.

Still further, alternative transgenic animal systems expressing human immunoglobulin genes are available in the art and can be used to raise anti-huCD40 antibodies described herein. For example, an alternative transgenic system referred to as the XENOMOUSE® (Abgenix, Inc.) can be used; such mice are described in, for example, U.S. Pat. Nos. 5,939,598; 6,075,181; 6,114,598; 6, 150,584 and 6,162,963 to Kucherlapati et al.

Moreover, alternative transchromosomic animal systems expressing human immunoglobulin genes are available in the art and can be used to raise anti-CD40 antibodies described herein. For example, mice carrying both a human heavy chain transchromosome and a human light chain transchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise anti-huCD40 antibodies described herein.

Additional mouse systems described in the art for raising human antibodies, e.g., human anti-huCD40 antibodies, include (i) the VELOCIMMUNE® mouse (Regeneron Pharmaceuticals, Inc.), in which the endogenous mouse heavy and light chain variable regions have been replaced, via homologous recombination, with human heavy and light chain variable regions, operatively linked to the endogenous mouse constant regions, such that chimeric antibodies (human V/mouse C) are raised in the mice, and then subsequently converted to fully human antibodies using standard recombinant DNA techniques; and (ii) the MEMO© mouse (Merus Biopharmaceuticals, Inc.), in which the mouse contains unrearranged human heavy chain variable regions but a single rearranged human common light chain variable region. Such mice, and use thereof to raise antibodies, are described in, for example, WO 2009/15777, US 2010/0069614, WO 2011/072204, WO 2011/097603, WO 2011/163311, WO 2011/163314, WO 2012/148873, US 2012/0070861 and US 2012/0073004.

Human monoclonal antibodies described herein can also be prepared using phage display methods for screening libraries of human immunoglobulin genes. Such phage display methods for isolating human antibodies are established in the art. See for example: U.S. Pat. Nos. 5,223,409; 5,403,484; and U.S. Pat. No. 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies described herein can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### Immunizations

To generate fully human antibodies to human CD40, mice or transgenic or transchromosomal mice containing human immunoglobulin genes (e.g., HCo12, HCo7 or KM mice) can be immunized with a purified or enriched preparation of the CD40 antigen and/or cells expressing CD40, as described for other antigens, for example, by Lonberg et al. (1994) Nature 368(6474): 856-859; Fishwild et al. (1996) Nature Biotechnology 14: 845-851 and WO 98/24884. Alternatively, mice can be immunized with DNA encoding human CD40. Preferably, the mice will be 6-16 weeks of age upon the first infusion. For example, a purified or enriched preparation (5-50 μg) of the recombinant human CD40 antigen can be used to immunize the mice intraperitoneally. In the event that immunizations using a purified or enriched preparation of the CD40 antigen do not result in antibodies, mice can also be immunized with cells expressing CD40, e.g., a cell line, to promote immune responses.

Cumulative experience with various antigens has shown that the HuMAb transgenic mice respond best when initially immunized intraperitoneally (IP) or subcutaneously (SC) with antigen in Ribi's adjuvant, followed by every other week IP/SC immunizations (up to a total of 10) with antigen in Ribi's adjuvant. The immune response can be monitored over the course of the immunization protocol with plasma samples being obtained by retroorbital bleeds. The plasma can be screened by ELISA and FACS (as described below), and mice with sufficient titers of anti-CD40 human immunoglobulin can be used for fusions. Mice can be boosted intravenously with antigen 3 days before sacrifice and removal of the spleen and lymph nodes. It is expected that 2-3 fusions for each immunization may need to be performed. Between 6 and 24 mice are typically immunized for each antigen. Usually, HCo7, HCo12, and KM strains are used. In addition, both HCo7 and HCo12 transgene can be bred together into a single mouse having two different human heavy chain transgenes (HCo7/HCo12).

### Generation of Hybridomas Producing Monoclonal Antibodies to CD40

To generate hybridomas producing monoclonal antibodies described herein, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to Sp2/0 nonsecreting mouse myeloma cells (ATCC, CRL 1581) with 50% PEG. Cells are plated at approximately 2×10⁵ in flat bottom microtiter plate, followed by a two week incubation in selective medium containing 10% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1× HAT (Sigma). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

### VI. Antibody Manufacture

### Generation of Transfectomas Producing Monoclonal Antibodies to CD40

Antibodies of the present invention, including both specific antibodies for which sequences are provided and other, related anti-CD40 antibodies, can be produced in a host cell transfectoma using, for example, a combination of recombinant DNA techniques and gene transfection methods as is well known in the art (Morrison, S. (1985) Science 229:1202).

For example, to express antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or both genes are inserted into the same expression vector. The antibody genes are inserted into the expression vector(s) by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, recombinant expression vectors may carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, CA (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies described herein in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13). Antibodies of the present invention can also be produced in glycoengineered strains of the yeast Pichia pastoris. Li et al. (2006) Nat. Biotechnol. 24:210.

Preferred mammalian host cells for expressing the recombinant antibodies described herein include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in R. J. Kaufman and P. A. Sharp (1982)Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

The N- and C-termini of antibody polypeptide chains of the present invention may differ from the expected sequence due to commonly observed post-translational modifications. For example, C-terminal lysine residues are often missing from antibody heavy chains. Dick et al. (2008) Biotechnol. Bioeng. 100:1132. N-terminal glutamine residues, and to a lesser extent glutamate residues, are frequently converted to pyroglutamate residues on both light and heavy chains of therapeutic antibodies. Dick et al. (2007) Biotechnol. Bioeng. 97:544; Liu et al. (2011) J Biol. Chem. 286:11211.

Amino acid sequences for various agonist anti-huCD40 antibodies of the present invention are provided in the Sequence Listing, which is summarized at Table 8. For the reasons mentioned above, the C-terminal lysine is not included in any of sequences in the Sequence Listing for heavy chains or heavy chain constant domains. However, in an alternative embodiment, each heavy chain for the anti-huCD40 antibodies of the present invention, and/or genetic construct encoding such antibodies or the heavy or light chains thereof, includes this additional lysine residue at the C-terminus of one or both of the heavy chains.

### VII. Assays

Antibodies described herein can be tested for binding to CD40 by, for example, standard ELISA. Briefly, microtiter plates are coated with purified CD40 at 1-2 μg/ml in PBS, and then blocked with 5% bovine serum albumin in PBS. Dilutions of antibody (e.g., dilutions of plasma from CD40-immunized mice) are added to each well and incubated for 1-2 hours at 37° C. The plates are washed with PBS/Tween and then incubated with secondary reagent (e.g., for human antibodies, or antibodies otherwise having a human heavy chain constant region, a goat-anti-human IgG Fc-specific polyclonal reagent) conjugated to horseradish peroxidase (HRP) for 1 hour at 37° C. After washing, the plates are developed with ABTS substrate (Moss Inc, product: ABTS-1000) and analyzed by a spectrophotometer at OD 415-495. Sera from immunized mice are then further screened by flow cytometry for binding to a cell line expressing human CD40, but not to a control cell line that does not express CD40. Briefly, the binding of anti-CD40 antibodies is assessed by incubating CD40 expressing CHO cells with the anti-CD40 antibody at 1:20 dilution. The cells are washed and binding is detected with a PE-labeled anti-human IgG Ab. Flow cytometric analyses are performed using a FACScan flow cytometry (Becton Dickinson, San Jose, CA). Preferably, mice that develop the highest titers will be used for fusions. Analogous experiments may be performed using anti-mouse detection antibodies if mouse anti-huCD40 antibodies are to be detected.

An ELISA as described above can be used to screen for antibodies and, thus, hybridomas that produce antibodies that show positive reactivity with the CD40 immunogen. Hybridomas that produce antibodies that bind, preferably with high affinity, to CD40 can then be subcloned and further characterized. One clone from each hybridoma, which retains the reactivity of the parent cells (by ELISA), can then be chosen for making a cell bank, and for antibody purification.

To purify anti-CD40 antibodies, selected hybridomas can be grown in two-liter spinner-flasks for monoclonal antibody purification. Supernatants can be filtered and concentrated before affinity chromatography with protein A-sepharose (Pharmacia, Piscataway, NJ). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The monoclonal antibodies can be aliquoted and stored at −80° C.

To determine if the selected anti-CD40 monoclonal antibodies bind to unique epitopes, each antibody can be biotinylated using commercially available reagents (Pierce, Rockford, IL). Biotinylated MAb binding can be detected with a streptavidin labeled probe. Competition studies using unlabeled monoclonal antibodies and biotinylated monoclonal antibodies can be performed using CD40 coated-ELISA plates as described above.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

To test the binding of monoclonal antibodies to live cells expressing CD40, flow cytometry can be used. Briefly, cell lines expressing membrane-bound CD40 (grown under standard growth conditions) are mixed with various concentrations of monoclonal antibodies in PBS containing 0.1% BSA at 4° C. for 1 hour. After washing, the cells are reacted with Phycoerythrin (PE)-labeled anti-IgG antibody under the same conditions as the primary antibody staining. The samples can be analyzed by FACScan instrument using light and side scatter properties to gate on single cells and binding of the labeled antibodies is determined. An alternative assay using fluorescence microscopy may be used (in addition to or instead of) the flow cytometry assay. Cells can be stained exactly as described above and examined by fluorescence microscopy. This method allows visualization of individual cells, but may have diminished sensitivity depending on the density of the antigen.

Anti-huCD40 antibodies can be further tested for reactivity with the CD40 antigen by Western blotting. Briefly, cell extracts from cells expressing CD40 can be prepared and subjected to sodium dodecyl sulfate polyacrylamide gel electrophoresis. After electrophoresis, the separated antigens will be transferred to nitrocellulose membranes, blocked with 20% mouse serum, and probed with the monoclonal antibodies to be tested. IgG binding can be detected using anti-IgG alkaline phosphatase and developed with BCIP/NBT substrate tablets (Sigma Chem. Co., St. Louis, MO).

Methods for analyzing binding affinity, cross-reactivity, and binding kinetics of various anti-CD40 antibodies include standard assays known in the art, for example, Biolayer Interferometry (BLI) analysis, and BIACORE® surface plasmon resonance (SPR) analysis using a BIACORE® 2000 SPR instrument (Biacore AB, Uppsala, Sweden).

In one embodiment, an antibody specifically binds to the extracellular region of human CD40. An antibody may specifically bind to a particular domain (e.g., a functional domain) within the extracellular domain of CD40. In certain embodiments, the antibody specifically binds to the extracellular region of human CD40 and the extracellular region of cynomolgus CD40. Preferably, an antibody binds to human CD40 with high affinity. V11I. Bispecific Molecules Antibodies described herein may be used for forming bispecific molecules. An anti-CD40 antibody, or antigen-binding fragments thereof, can be derivatized or linked to another functional molecule, e.g., another peptide or protein (e.g., another antibody or ligand for a receptor) to generate a bispecific molecule that binds to at least two different binding sites or target molecules. The antibody described herein may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule described herein, an antibody described herein can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, provided herein are bispecific molecules comprising at least one first binding specificity for CD40 and a second binding specificity for a second target epitope. In an embodiment described herein in which the bispecific molecule is multispecific, the molecule can further include a third binding specificity.

In one embodiment, the bispecific molecules described herein comprise as a binding specificity at least one antibody, or an antibody fragment thereof, including, e.g. an Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which is expressly incorporated by reference.

While human monoclonal antibodies are preferred, other antibodies that can be employed in the bispecific molecules described herein are murine, chimeric and humanized monoclonal antibodies.

The bispecific molecules described herein can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand×Fab fusion protein. A bispecific molecule described herein can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed using art-recognized methods, such as enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

### IX. Compositions

Further provided are compositions, e.g., a pharmaceutical compositions, containing one or more anti-CD40 antibodies, or antigen-binding fragment(s) thereof, as described herein, formulated together with a pharmaceutically acceptable carrier. Such compositions may include one or a combination of (e.g., two or more different) antibodies, or immunoconjugates or bispecific molecules described herein. For example, a pharmaceutical composition described herein can comprise a combination of antibodies (or immunoconjugates or bispecifics) that bind to different epitopes on the target antigen or that have complementary activities.

In certain embodiments, a composition comprises an anti-CD40 antibody at a concentration of at least 1 mg/ml, 5 mg/ml, 10 mg/ml, 50 mg/ml, 100 mg/ml, 150 mg/ml, 200 mg/ml, or at 1-300 mg/ml or 100-300 mg/ml.

Pharmaceutical compositions described herein also can be administered in combination therapy, i.e., combined with other agents. For example, the combination therapy can include an anti-CD40 antibody described herein combined with at least one other anti-cancer and/or T-cell stimulating (e.g., activating) agent. Examples of therapeutic agents that can be used in combination therapy are described in greater detail below in the section on uses of the antibodies described herein.

In some embodiments, therapeutic compositions disclosed herein can include other compounds, drugs, and/or agents used for the treatment of cancer. Such compounds, drugs, and/or agents can include, for example, chemotherapy drugs, small molecule drugs or antibodies that stimulate the immune response to a given cancer. In some instances, therapeutic compositions can include, for example, one or more of an anti-CTLA-4 antibody, an anti-PD-1 antibody, an anti-PD-L1 antibody, an anti-TIGIT antibody, an anti-OX40 (also known as CD134, TNFRSF4, ACT35 and/or TXGP1L) antibody, an anti-LAG-3 antibody, an anti-CD73 antibody, an anti-CD137 antibody, an anti-CD27 antibody, an anti-CSF-1R antibody, a TLR agonist, or a small molecule antagonist of IDO or TGFβ.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, may be coated in a material to protect the compound from the action of acids and other natural conditions that may inactivate the compound.

The pharmaceutical compounds described herein may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition described herein also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient that can be combined with a carrier material to produce a single dosage form will vary depending upon the subject being treated, and the particular mode of administration. The amount of active ingredient that can be combined with a carrier material to produce a single dosage form will generally be that amount of the composition that produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms described herein are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months.

In some methods, two or more monoclonal antibodies with different binding specificities are administered simultaneously, in which case the dosage of each antibody administered falls within the ranges indicated. A therapeutic antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

An antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can optionally be administered a prophylactic regime, although in many immune-oncology indications continued treatment is not necessary.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient. The selected dosage level will depend upon a variety of pharmacokinetic factors including the activity of the particular compositions described herein employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-CD40 antibody described herein preferably results in a decrease in severity of disease symptoms, an increase in frequency and duration of disease symptom-free periods, or a prevention of impairment or disability due to the disease affliction. In the context of cancer, a therapeutically effective dose preferably prevents further deterioration of physical symptoms associated with cancer. Symptoms of cancer are well-known in the art and include, for example, unusual mole features, a change in the appearance of a mole, including asymmetry, border, color and/or diameter, a newly pigmented skin area, an abnormal mole, darkened area under nail, breast lumps, nipple changes, breast cysts, breast pain, death, weight loss, weakness, excessive fatigue, difficulty eating, loss of appetite, chronic cough, worsening breathlessness, coughing up blood, blood in the urine, blood in stool, nausea, vomiting, liver metastases, lung metastases, bone metastases, abdominal fullness, bloating, fluid in peritoneal cavity, vaginal bleeding, constipation, abdominal distension, perforation of colon, acute peritonitis (infection, fever, pain), pain, vomiting blood, heavy sweating, fever, high blood pressure, anemia, diarrhea, jaundice, dizziness, chills, muscle spasms, colon metastases, lung metastases, bladder metastases, liver metastases, bone metastases, kidney metastases, and pancreatic metastases, difficulty swallowing, and the like. Therapeutic efficacy may be observable immediately after the first administration of an agonistic anti-huCD40 mAb of the present invention, or it may only be observed after a period of time and/or a series of doses. Such delayed efficacy my only be observed after several months of treatment, up to 6, 9 or 12 months. It is critical not to decide prematurely that an agonistic anti-huCD40 mAb of the present invention lacks therapeutically efficacy in light of the delayed efficacy exhibited by some immune-oncology agents.

A therapeutically effective dose may prevent or delay onset of cancer, such as may be desired when early or preliminary signs of the disease are present. Laboratory tests utilized in the diagnosis of cancer include chemistries (including the measurement of soluble CD40 or CD40L levels)(Hock et al. (2006) Cancer 106:2148; Chung & Lim (2014) J Trans. Med. 12:102), hematology, serology and radiology. Accordingly, any clinical or biochemical assay that monitors any of the foregoing may be used to determine whether a particular treatment is a therapeutically effective dose for treating cancer. One of ordinary skill in the art would be able to determine such amounts based on such factors as the subject's size, the severity of the subject's symptoms, and the particular composition or route of administration selected.

A composition described herein can be administered via one or more routes of administration using one or more of a variety of methods known in the art. As will be appreciated by the skilled artisan, the route and/or mode of administration will vary depending upon the desired results. Preferred routes of administration for antibodies described herein include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody described herein can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition described herein can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules for use with anti-huCD40 antibodies described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the anti-huCD40 antibodies described herein can be formulated to ensure proper distribution in vivo. For example, the blood-brain barrier (BBB) excludes many highly hydrophilic compounds. To ensure that the therapeutic compounds described herein cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties that are selectively transported into specific cells or organs, thus enhance targeted drug delivery (see, e.g., V. V. Ranade (1989) J Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBSLett. 357:140; M. Owais et al. (1995) Antimicrob. Agents Chemother. 39:180); surfactant protein A receptor (Briscoe et al. (1995) Am. J. Physiol. 1233:134); p120 (Schreier et al. (1994) J. Biol. Chem. 269:9090); see also K. Keinanen; M. L. Laukkanen (1994) FEBSLett. 346:123; J. J. Killion; I. J. Fidler (1994) Immunomethods 4:273.

### X. Uses and Methods

The antibodies, antibody compositions and methods described herein have numerous in vitro and in vivo utilities involving, for example, enhancement of immune response by agonizing CD40 signaling. In a preferred embodiment, the antibodies described herein are human or humanized antibodies. For example, anti-huCD40 antibodies described herein can be administered to cells in culture, in vitro or ex vivo, or to human subjects, e.g., in vivo, to enhance immunity in a variety of diseases. Accordingly, provided herein are methods of modifying an immune response in a subject comprising administering to the subject an antibody, or antigen-binding fragment thereof, described herein such that the immune response in the subject is enhanced, stimulated or up-regulated.

Preferred subjects include human patients in whom enhancement of an immune response would be desirable. The methods are particularly suitable for treating human patients having a disorder that can be treated by augmenting an immune response (e.g. the T-cell mediated immune response). In a particular embodiment, the methods are particularly suitable for treatment of cancer in vivo. To achieve antigen-specific enhancement of immunity, anti-huCD40 antibodies described herein can be administered together with an antigen of interest or the antigen may already be present in the subject to be treated (e.g., a tumor-bearing or virus-bearing subject). When antibodies to CD40 are administered together with another agent, the two can be administered separately or simultaneously.

Also encompassed are methods for detecting the presence of human CD40 antigen in a sample, or measuring the amount of human CD40 antigen, comprising contacting the sample, and a control sample, with a human monoclonal antibody, or an antigen binding fragment thereof, that specifically binds to human CD40, under conditions that allow for formation of a complex between the antibody or fragment thereof and human CD40. The formation of a complex is then detected, wherein a difference complex formation between the sample compared to the control sample is indicative the presence of human CD40 antigen in the sample. Moreover, the anti-CD40 antibodies described herein can be used to purify human CD40 via immunoaffinity purification.

Given the ability of anti-huCD40 antibodies described herein to enhance co-stimulation of T cell responses, e.g., antigen-specific T cell responses, provided herein are in vitro and in vivo methods of using the antibodies described herein to stimulate, enhance or upregulate antigen-specific T cell responses, e.g., anti-tumor T cell responses. CD4⁺ and CD8⁺ T cell responses can be enhanced using anti-CD40 antibodies. The T cells can be T_{eff} cells, e.g., CD4+ T_{eff} cells, CD8+ T_{eff} cells, T helper (Tₕ) cells and T cytotoxic (T_{c}) cells.

Further encompassed are methods of enhancing an immune response (e.g., an antigen-specific T cell response) in a subject comprising administering an anti-huCD40 antibody described herein to the subject such that an immune response (e.g., an antigen-specific T cell response) in the subject is enhanced. In a preferred embodiment, the subject is a tumor-bearing subject and an immune response against the tumor is enhanced. A tumor may be a solid tumor or a liquid tumor, e.g., a hematological malignancy. In certain embodiments, a tumor is an immunogenic tumor. In certain embodiments, a tumor is non-immunogenic. In certain embodiments, a tumor is PD-L1 positive. In certain embodiments a tumor is PD-L1 negative. A subject may also be a virus-bearing subject and an immune response against the virus is enhanced.

Further provided are methods for inhibiting growth of tumor cells in a subject comprising administering to the subject an anti-huCD40 antibody described herein such that growth of the tumor is inhibited in the subject. Also provided are methods of treating chronic viral infection in a subject comprising administering to the subject an anti-huCD40 antibody described herein such that the chronic viral infection is treated in the subject.

In certain embodiments, an anti-huCD40 antibody is given to a subject as an adjunctive therapy. Treatments of subjects having cancer with an anti-huCD40 antibody may lead to a long-term durable response relative to the current standard of care; long term survival of at least 1, 2, 3, 4, 5, 10 or more years, recurrence free survival of at least 1, 2, 3, 4, 5, or 10 or more years. In certain embodiments, treatment of a subject having cancer with an anti-huCD40 antibody prevents recurrence of cancer or delays recurrence of cancer by, e.g., 1, 2, 3, 4, 5, or 10 or more years. An anti-CD40 treatment can be used as a primary or secondary line of treatment.

These and other methods described herein are discussed in further detail below.

### Cancer

Provided herein are methods for treating a subject having cancer, comprising administering to the subject an anti-huCD40 antibody described herein, such that the subject is treated, e.g., such that growth of cancerous tumors is inhibited or reduced and/or that the tumors regress. An anti-huCD40 antibody can be used alone to inhibit the growth of cancerous tumors. Alternatively, an anti-huCD40 antibody can be used in conjunction with another agent, e.g., other immunogenic agents, standard cancer treatments, or other antibodies, as described below. Combination with an inhibitor of PD-1, such as an anti-PD-1 or anti-PD-L1 antibody, is also provided. See. e.g., Ellmark et al. (2015) OncoImmunology 4:7 el 011484.

Accordingly, provided herein are methods of treating cancer, e.g., by inhibiting growth of tumor cells, in a subject, comprising administering to the subject a therapeutically effective amount of an anti-huCD40 antibody described herein, e.g., a humanized form of 12D6, 5F11, 8E8, 5G7 or 19G3, or antigen-binding fragment thereof. The antibody may be a humanized anti-huCD40 antibody (such as any of the humanized anti-huCD40 antibodies described herein), a human chimeric anti-huCD40 antibody, or a humanized non-human anti-huCD40 antibody, e.g., a human, chimeric or humanized anti-huCD40 antibody that competes for binding with, or binds to the same epitope as, at least one of the anti-huCD40 antibodies specifically described herein.

Cancers whose growth may be inhibited using the antibodies of the invention include cancers typically responsive to immunotherapy. Non-limiting examples of cancers for treatment include squamous cell carcinoma, small-cell lung cancer, non-small cell lung cancer, squamous non-small cell lung cancer (NSCLC), non NSCLC, glioma, gastrointestinal cancer, renal cancer (e.g. clear cell carcinoma), ovarian cancer, liver cancer, colorectal cancer, endometrial cancer, kidney cancer (e.g., renal cell carcinoma (RCC)), prostate cancer (e.g. hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally-induced cancers including those induced by asbestos, virus-related cancers (e.g., human papilloma virus (HPV)-related tumor), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. The methods described herein may also be used for treatment of metastatic cancers, refractory cancers (e.g., cancers refractory to previous immunotherapy, e.g., with a blocking CTLA-4 or PD-1 antibody), and recurrent cancers.

Notwithstanding the above, the agonist anti-huCD40 antibodies of the present invention will not find use in treating hematologic cancers with CD40 expression, which might be exacerbated by treatment with a CD40 agonist. Certain cancers may be known to express CD40 and thus be subject to such exacerbation, and thus may be categorically excluded. In other embodiments specific tumor samples are tested for expression of CD40 and are excluded from therapy with the agonist anti-huCD40 antibodies of the present invention based on the test results.

An anti-huCD40 antibody can be administered as a monotherapy, or as the only immunostimulating therapy, or it can be combined with an immunogenic agent in a cancer vaccine strategy, such as cancerous cells, purified tumor antigens (including recombinant proteins, peptides, and carbohydrate molecules), cells, and cells transfected with genes encoding immune stimulating cytokines (He et al. (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF. Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita et al. (eds.), 1997, Cancer: Principles and Practice of Oncology, Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination. Dranoff et al. (1993) Proc. Natl. Acad. Sci. USA 90: 3539-43.

The study of gene expression and large scale gene expression patterns in various tumors has led to the definition of so called tumor specific antigens. Rosenberg, S A (1999) Immunity 10: 281-7. In many cases, these tumor specific antigens are differentiation antigens expressed in the tumors and in the cell from which the tumor arose, for example melanocyte antigens gp100, MAGE antigens, and Trp-2. More importantly, many of these antigens can be shown to be the targets of tumor specific T cells found in the host. CD40 agonists can be used in conjunction with a collection of recombinant proteins and/or peptides expressed in a tumor in order to generate an immune response to these proteins. These proteins are normally viewed by the immune system as self antigens and are therefore tolerant to them. The tumor antigen can include the protein telomerase, which is required for the synthesis of telomeres of chromosomes and which is expressed in more than 85% of human cancers and in only a limited number of somatic tissues (Kim et al. (1994) Science 266: 2011-2013). Tumor antigen can also be “neo-antigens” expressed in cancer cells because of somatic mutations that alter protein sequence or create fusion proteins between two unrelated sequences (i.e., bcr-abl in the Philadelphia chromosome), or idiotype from B cell tumors.

Other tumor vaccines can include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen that can be used in conjunction with CD40 inhibition is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot & Srivastava (1995) Science 269:1585-1588; Tamura et al. (1997) Science 278:117-120).

Dendritic cells (DC) are potent antigen presenting cells that can be used to prime antigen-specific responses. DC's can be produced ex vivo and loaded with various protein and peptide antigens as well as tumor cell extracts (Nestle et al. (1998) Nature Medicine 4: 328-332). DCs can also be transduced by genetic means to express these tumor antigens as well. DCs have also been fused directly to tumor cells for the purposes of immunization (Kugler et al. (2000) Nature Medicine 6:332-336). As a method of vaccination, DC immunization can be effectively combined with CD40 agonism to activate (unleash) more potent anti-tumor responses.

Agonism of CD40 can also be combined with standard cancer treatments (e.g., surgery, radiation, and chemotherapy). Agonism of CD40 can be effectively combined with chemotherapeutic regimes. In these instances, it may be possible to reduce the dose of chemotherapeutic reagent administered (Mokyr et al. (1998) Cancer Research 58: 5301-5304). An example of such a combination is an anti-huCD40 antibody in combination with decarbazine for the treatment of melanoma. Another example of such a combination is an anti-huCD40 antibody in combination with interleukin-2 (IL-2) for the treatment of melanoma. The scientific rationale behind the combined use of CD40 agonists and chemotherapy is that cell death, which is a consequence of the cytotoxic action of most chemotherapeutic compounds, should result in increased levels of tumor antigen in the antigen presentation pathway. Other combination therapies that may result in synergy with CD40 agonism through cell death are radiation, surgery, and hormone deprivation. Each of these protocols creates a source of tumor antigen in the host. Angiogenesis inhibitors can also be combined with CD40 agonists. Inhibition of angiogenesis leads to tumor cell death which may feed tumor antigen into host antigen presentation pathways.

The anti-huCD40 antibodies described herein can also be used in combination with bispecific antibodies that target Fcα or Fcγ receptor-expressing effectors cells to tumor cells (see, e.g., U.S. Pat. Nos. 5,922,845 and 5,837,243). Bispecific antibodies can be used to target two separate antigens. For example anti-Fc receptor/anti tumor antigen (e.g., Her-2/neu) bispecific antibodies have been used to target macrophages to sites of tumor. This targeting may more effectively activate tumor specific responses. The T cell arm of these responses would be augmented by agonism of CD40. Alternatively, antigen may be delivered directly to DCs by the use of bispecific antibodies that bind to tumor antigen and a dendritic cell specific cell surface marker.

Tumors evade host immune surveillance by a large variety of mechanisms. Many of these mechanisms may be overcome by the inactivation of immunosuppressive proteins expressed by the tumors. These include among others TGF-β (Kehrl et al. (1986) J Exp. Med. 163: 1037-1050), IL-10 (Howard & O'Garra (1992) Immunology Today 13: 198-200), and Fas ligand (Hahne et al. (1996) Science 274: 1363-1365). Antibodies to each of these entities can be used in combination with anti-huCD40 antibodies to counteract the effects of the immunosuppressive agent and favor tumor immune responses by the host.

Anti-CD40 antibodies are able to substitute effectively for T cell helper activity. Ridge et al. (1998) Nature 393: 474-478. Activating antibodies to T cell costimulatory molecules such as CTLA-4 (e.g., U.S. Pat. No. 5,811,097), OX-40 (Weinberg et al. (2000) Immunol. 164: 2160-2169), CD137/4-1BB (Melero et al. (1997) Nature Medicine 3: 682-685 (1997), and ICOS (Hutloff et al. (1999) Nature 397: 262-266) may also provide for increased levels of T cell activation. Inhibitors of PD1 or PD-L1 may also be used in conjunction with anti-huCD40 antibodies.

There are also several experimental treatment protocols that involve ex vivo activation and expansion of antigen specific T cells and adoptive transfer of these cells into recipients in order to stimulate antigen-specific T cells against tumor (Greenberg & Riddell (1999) Science 285: 546-51). These methods can also be used to activate T cell responses to infectious agents such as CMV. Ex vivo activation in the presence of anti-CD40 antibodies can increase the frequency and activity of the adoptively transferred T cells.

### Chronic Viral Infections

In another aspect, the invention described herein provides a method of treating an infectious disease in a subject comprising administering to the subject an anti-huCD40 antibody, or antigen-binding fragment thereof, such that the subject is treated for the infectious disease.

Similar to its application to tumors as discussed above, antibody-mediated CD40 agonism can be used alone, or as an adjuvant, in combination with vaccines, to enhance the immune response to pathogens, toxins, and self-antigens. Examples of pathogens for which this therapeutic approach can be particularly useful, include pathogens for which there is currently no effective vaccine, or pathogens for which conventional vaccines are less than completely effective. These include, but are not limited to HIV, Hepatitis (A, B, & C), Influenza, Herpes, Giardia, Malaria, Leishmania, Staphylococcus aureus, Pseudomonas aeruginosa. CD40 agonism is particularly useful against established infections by agents such as HIV that present altered antigens over the course of the infections. These novel epitopes are recognized as foreign at the time of anti-human CD40 antibody administration, thus provoking a strong T cell response.

Some examples of pathogenic viruses causing infections treatable by methods described herein include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Some examples of pathogenic bacteria causing infections treatable by methods described herein include chlamydia, rickettsial bacteria, mycobacteria, staphylococci, streptococci, pneumonococci, meningococci and gonococci, klebsiella, proteus, serratia, pseudomonas, legionella, diphtheria, salmonella, bacilli, cholera, tetanus, botulism, anthrax, plague, leptospirosis, and Lymes disease bacteria.

Some examples of pathogenic fungi causing infections treatable by methods described herein include Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

Some examples of pathogenic parasites causing infections treatable by methods described herein include Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondii, Nippostrongylus brasiliensis.

In all of the above methods, CD40 agonism can be combined with other forms of immunotherapy such as cytokine treatment (e.g., interferons, GM-CSF, G-CSF, IL-2), or bispecific antibody therapy, which provides for enhanced presentation of tumor antigens. See, e.g., Holliger (1993) Proc. Natl. Acad. Sci. USA 90:6444-6448; Poljak (1994) Structure 2:1121-1123.

### Vaccine Adjuvants

Anti-huCD40 antibodies described herein can be used to enhance antigen-specific immune responses by co-administration of an anti-huCD40 antibody with an antigen of interest, e.g., a vaccine. Accordingly, provided herein are methods of enhancing an immune response to an antigen in a subject, comprising administering to the subject: (i) the antigen; and (ii) an anti-huCD40 antibody, or antigen-binding fragment thereof, such that an immune response to the antigen in the subject is enhanced. The antigen can be, for example, a tumor antigen, a viral antigen, a bacterial antigen or an antigen from a pathogen. Non-limiting examples of such antigens include those discussed in the sections above, such as the tumor antigens (or tumor vaccines) discussed above, or antigens from the viruses, bacteria or other pathogens described above.

Suitable routes of administering the antibody compositions (e.g., human monoclonal antibodies, multispecific and bispecific molecules and immunoconjugates) described herein in vivo and in vitro are well known in the art and can be selected by those of ordinary skill. For example, the antibody compositions can be administered by injection (e.g., intravenous or subcutaneous). Suitable dosages of the molecules used will depend on the age and weight of the subject and the concentration and/or formulation of the antibody composition.

As previously described, anti-huCD40 antibodies described herein can be co-administered with one or other more therapeutic agents, e.g., a cytotoxic agent, a radiotoxic agent or an immunosuppressive agent. The antibody can be linked to the agent (as an immuno-complex) or can be administered separate from the agent. In the latter case (separate administration), the antibody can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil, dacarbazine and cyclophosphamide hydroxyurea which, by themselves, are only effective at levels which are toxic or subtoxic to a patient. Cisplatin is intravenously administered as a 100 mg/ml dose once every four weeks and adriamycin is intravenously administered as a 60-75 mg/ml dose once every 21 days. Co-administration of anti-CD40 antibodies, or antigen binding fragments thereof, described herein with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells that would render them unreactive with the antibody.

Also within the scope described herein are kits comprising the antibody compositions described herein (e.g., human antibodies, bispecific or multispecific molecules, or immunoconjugates) and instructions for use. The kit can further contain at least one additional reagent, or one or more additional human antibodies described herein (e.g., a human antibody having a complementary activity that binds to an epitope in CD40 antigen distinct from the first human antibody). Kits typically include a label indicating the intended use of the contents of the kit. The term label includes any writing, or recorded material supplied on or with the kit, or that otherwise accompanies the kit.

### Combination Therapies

In addition to the combinations therapies provided above, anti-CD40 antibodies described herein can also be used in combination therapy, e.g., for treating cancer, as described below.

The present invention provides methods of combination therapy in which an anti-huCD40 antibody is co-administered with one or more additional agents, e.g., antibodies, that are effective in stimulating immune responses to thereby further enhance, stimulate or upregulate immune responses in a subject.

Generally, an anti-huCD40 antibody described herein can be combined with (i) an agonist of another co-stimulatory receptor and/or (ii) an antagonist of an inhibitory signal on T cells, either of which results in amplifying antigen-specific T cell responses (immune checkpoint regulators). Most of the co-stimulatory and co-inhibitory molecules are members of the immunoglobulin super family (IgSF), and anti-CD40 antibodies described herein may be administered with an agent that targets a member of the IgSF family to increase an immune response. One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which include CD40 and CD40L, OX-40, OX-40L, CD70, CD27L, CD30, CD30L, 4-1BBL, CD137/4-1BB, TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α 1β2, FAS, FASL, RELT, DR6, TROY, NGFR (see, e.g., Tansey (2009) Drug Discovery Today 00:1).

In another aspect, anti-huCD40 antibodies can be used in combination with antagonists of cytokines that inhibit T cell activation (e.g., IL-6, IL-10, TGF-β, VEGF); or other “immunosuppressive cytokines,” or cytokines that stimulate T cell activation, for stimulating an immune response, e.g., for treating proliferative diseases, such as cancer.

In one aspect, T cell responses can be stimulated by a combination of the anti-huCD40 mAbs of the present invention and one or more of (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

Exemplary agents that modulate one of the above proteins and may be combined with agonist anti-huCD40 antibodies, e.g., those described herein, for treating cancer, include: YERVOY®/ipilimumab or tremelimumab (to CTLA-4), galiximab (to B7.1), BMS-936558 (to PD-1), pidilizumab/CT-011 (to PD-1), KEYTRUDA®/pembrolizumab/MK-3475 (to PD-1), AMP224 (to B7-DC/PD-L2), BMS-936559 (to B7-H1), MPDL3280A (to B7-H1), MEDI-570 (to ICOS), AMG557 (to B7H2), MGA271 (to B7H3-WO 11/109400), IMP321 (to LAG-3), urelumab/BMS-663513 and PF-05082566 (to CD137/4-1BB), varlilumab/CDX-1127 (to CD27), MEDI-6383 and MEDI-6469 (to OX40), RG-7888 (to OX40L-WO 06/029879), Atacicept (to TACI), muromonab-CD3 (to CD3), ipilumumab (to CTLA-4).

Other molecules that can be combined with agonist anti-huCD40 antibodies for the treatment of cancer include antagonists of inhibitory receptors on NK cells or agonists of activating receptors on NK cells. For example, agonist anti-huCD40 antibodies can be combined with antagonists of KIR (e.g., lirilumab).

Yet other agents for combination therapies include agents that inhibit or deplete macrophages or monocytes, including but not limited to CSF-1R antagonists such as CSF-1R antagonist antibodies including RG7155 (WO 11/70024, WO 11/107553, WO 11/131407, WO 13/87699, WO 13/119716, WO 13/132044) or FPA-008 (WO 11/140249; WO 13/169264; WO 14/036357).

Generally, agonist anti-huCD40 antibodies described herein can be used together with one or more of agonistic agents that ligate positive co-stimulatory receptors, blocking agents that attenuate signaling through inhibitory receptors, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

Provided herein are methods for stimulating an immune response in a subject comprising administering to the subject a CD40 agonist, e.g., an antibody, and one or more additional immunostimulatory antibodies, such as a PD-1 antagonist, e.g., antagonist antibody, a PD-L1 antagonist, e.g., antagonist antibody, a CTLA-4 antagonist, e.g., antagonist antibody and/or a LAG3 antagonist, e.g., an antagonist antibody, such that an immune response is stimulated in the subject, for example to inhibit tumor growth or to stimulate an anti-viral response. In one embodiment, the subject is administered an agonist anti-huCD40 antibody and an antagonist anti-PD-1 antibody. In one embodiment, the subject is administered an agonist anti-huCD40 antibody and an antagonist anti-PD-L1 antibody. In one embodiment, the subject is administered an agonist anti-huCD40 antibody and an antagonist anti-CTLA-4 antibody. In one embodiment, the at least one additional immunostimulatory antibody (e.g., an antagonist anti-PD-1, an antagonist anti-PD-L1, an antagonist anti-CTLA-4 and/or an antagonist anti-LAG3 antibody) is a human antibody. Alternatively, the at least one additional immunostimulatory antibody can be, for example, a chimeric or humanized antibody (e.g., prepared from a mouse anti-PD-1, anti-PD-L1, anti-CTLA-4 and/or anti-LAG3 antibody).

Provided herein are methods for treating a hyperproliferative disease (e.g., cancer), comprising administering an agonist anti-huCD40 antibody and an antagonist PD-1 antibody to a subject. In certain embodiments, the agonist anti-huCD40 antibody is administered at a subtherapeutic dose, the anti-PD-1 antibody is administered at a subtherapeutic dose, or both are administered at a subtherapeutic dose, wherein the subtherapeutic designation is with reference to monotherapy with the agent in question. Also provided herein are methods for altering an adverse event associated with treatment of a hyperproliferative disease with an immunostimulatory agent, comprising administering an agonist anti-huCD40 antibody and a subtherapeutic dose of anti-PD-1 antibody to a subject. In certain embodiments, the subject is human. In certain embodiments, the anti-PD-1 antibody is a human sequence monoclonal antibody and the agonist anti-huCD40 antibody is a humanized monoclonal antibody, such as an antibody comprising the CDRs or variable regions of the antibodies disclosed herein.

Suitable PD-1 antagonists for use in the methods described herein, include, without limitation, ligands, antibodies (e.g., monoclonal antibodies and bispecific antibodies), and multivalent agents. In one embodiment, the PD-1 antagonist is a fusion protein, e.g., an Fc fusion protein, such as AMP-244. In one embodiment, the PD-1 antagonist is an anti-PD-1 or anti-PD-L1 antibody.

An exemplary anti-PD-1 antibody is OPDIVO®/nivolumab (BMS-936558) or an antibody that comprises the CDRs or variable regions of one of antibodies 17D8, 2D3, 4H1, 5C4, 7D3, 5F4 and 4A11 described in WO 2006/121168. In certain embodiments, an anti-PD-1 antibody is MK-3475 (KEYTRUDA®/pembrolizumab/formerly lambrolizumab) described in WO 2012/145493; AMP-514/MEDI-0680 described in WO 2012/145493; and CT-011 (pidilizumab; previously CT-AcTibody or BAT; see, e.g., Rosenblatt et al. (2011) J. Immunotherapy 34:409). Further known PD-1 antibodies and other PD-1 inhibitors include those described in WO 2009/014708, WO 03/099196, WO 2009/114335, WO 2011/066389, WO 2011/161699, WO 2012/145493, U.S. Pat. Nos. 7,635,757 and 8,217,149, and U.S. Patent Publication No. 2009/0317368. Any of the anti-PD-1 antibodies disclosed in WO 2013/173223 may also be used. An anti-PD-1 antibody that competes for binding with, and/or binds to the same epitope on PD-1 as, as one of these antibodies may also be used in combination treatments.

In certain embodiments, the anti-PD-1 antibody binds to human PD-1 with a K_{D} of 5×10⁻⁸ M or less, binds to human PD-1 with a K_{D} of 1×10⁻⁸ M or less, binds to human PD-1 with a K_{D} of 5×10⁻⁹ M or less, or binds to human PD-1 with a K_{D} of between 1×10⁻⁸M and 1×10⁻¹⁰ M or less.

Provided herein are methods for treating a hyperproliferative disease (e.g., cancer), comprising administering an agonist anti-huCD40 antibody and an antagonist PD-L1 antibody to a subject. In certain embodiments, the agonist anti-huCD40 antibody is administered at a subtherapeutic dose, the anti-PD-L1 antibody is administered at a subtherapeutic dose, or both are administered at a subtherapeutic dose. Provided herein are methods for altering an adverse event associated with treatment of a hyperproliferative disease with an immunostimulatory agent, comprising administering an agonist anti-huCD40 antibody and a subtherapeutic dose of anti-PD-L1 antibody to a subject. In certain embodiments, the subject is human. In certain embodiments, the anti-PD-L1 antibody is a human sequence monoclonal antibody and the agonist anti-huCD40 antibody is a humanized monoclonal antibody, such as an antibody comprising the CDRs or variable regions of the antibodies disclosed herein.

In one embodiment, the anti-PD-L1 antibody is BMS-936559 (referred to as 12A4 in WO 2007/005874 and U.S. Pat. No. 7,943,743), MSB0010718C (WO 2013/79174), or an antibody that comprises the CDRs or variable regions of 3G10, 12A4, 10A5, 5F8, 10H10, 1B12, 7H1, 11E6, 12B7 and 13G4, which are described in PCT Publication WO 07/005874 and U.S. Pat. No. 7,943,743. In certain embodiment an anti-PD-L1 antibody is MED14736 (also known as Anti-B7-H1) or MPDL3280A (also known as RG7446). Any of the anti-PD-L1 antibodies disclosed in WO 2013/173223, WO 2011/066389, WO 2012/145493, U.S. Pat. Nos. 7,635,757 and 8,217,149 and U.S. Publication No. 2009/145493 may also be used. Anti-PD-L1 antibodies that compete with and/or bind to the same epitope as that of any of these antibodies may also be used in combination treatments.

In yet further embodiment, the agonist anti-huCD40 antibody of the present invention is combined with an antagonist of PD-1/PD-L1 signaling, such as a PD-1 antagonist or a PD-L1 antagonist, in combination with a third immunotherapeutic agent. In one embodiment the third immunotherapeutic agent is a GITR antagonist or an OX-40 antagonist, such as the anti-GITR or anti-OX40 antibodies disclosed herein.

In another aspect, the immuno-oncology agent is a GITR agonist, such as an agonistic GITR antibody. Suitable GITR antibodies include, for example, BMS-986153, BMS-986156, TRX-518 (WO 06/105021, WO 09/009116) and MK-4166 (WO 11/028683).

In another aspect, the immuno-oncology agent is an IDO antagonist. Suitable IDO antagonists include, for example, INCB-024360 (WO 2006/122150, WO 07/75598, WO 08/36653, WO 08/36642), indoximod, or NLG-919 (WO 09/73620, WO 09/1156652, WO 11/56652, WO 12/142237).

Provided herein are methods for treating a hyperproliferative disease (e.g., cancer), comprising administering an agonist anti-huCD40 antibody described herein and a CTLA-4 antagonist antibody to a subject. In certain embodiments, the agonist anti-huCD40 antibody is administered at a subtherapeutic dose, the anti-CTLA-4 antibody is administered at a subtherapeutic dose, or both are administered at a subtherapeutic dose, wherein the subtherapeutic designation is with reference to monotherapy with the agent in question. Provided herein are methods for altering an adverse event associated with treatment of a hyperproliferative disease with an immunostimulatory agent, comprising administering an agonist anti-huCD40 antibody and a subtherapeutic dose of anti-CTLA-4 antibody to a subject. In certain embodiments, the subject is human. In certain embodiments, the anti-CTLA-4 antibody is an antibody selected from the group consisting of: YERVOY® (ipilimumab or antibody 10D1, described in PCT Publication WO 01/14424), tremelimumab (formerly ticilimumab, CP-675,206), and the anti-CTLA-4 antibodies described in the following publications: WO 98/42752; WO 00/37504; U.S. Pat. No. 6,207,156; Hurwitz et al. (1998) Proc. Natl. Acad. Sci. USA 95(17):10067-10071; Camacho et al. (2004) J. Clin. Oncology 22(145): Abstract No. 2505 (antibody CP-675206); and Mokyr et al. (1998) Cancer Res. 58:5301-5304. Any of the anti-CTLA-4 antibodies disclosed in WO 2013/173223 may also be used.

Provided herein are methods for treating a hyperproliferative disease (e.g., cancer), comprising administering an agonist anti-huCD40 antibody and an anti-LAG-3 antibody to a subject. In further embodiments, the agonist anti-huCD40 antibody is administered at a subtherapeutic dose, the anti-LAG-3 antibody is administered at a subtherapeutic dose, or both are administered at a subtherapeutic dose. Provided herein are methods for altering an adverse event associated with treatment of a hyperproliferative disease with an immunostimulatory agent, comprising administering an agonist anti-huCD40 antibody and a subtherapeutic dose of anti-LAG-3 antibody to a subject. In certain embodiments, the subject is human. In certain embodiments, the anti-LAG-3 antibody is a human sequence monoclonal antibody and the agonist anti-huCD40 antibody is a humanized monoclonal antibody, such as an antibody comprising the CDRs or variable regions of the antibodies disclosed herein. Examples of anti-LAG3 antibodies include antibodies comprising the CDRs or variable regions of antibodies 25F7, 26H10, 25E3, 8B7, 11F2 or 17E5, which are described in U.S. Patent Publication No. US 2011/0150892 and WO 2014/008218. In one embodiment, an anti-LAG-3 antibody is BMS-986016. Other art recognized anti-LAG-3 antibodies that can be used include IMP731 described in US 2011/007023. IMP-321 may also be used. Anti-LAG-3 antibodies that compete with and/or bind to the same epitope as that of any of these antibodies may also be used in combination treatments.

In certain embodiments, the anti-LAG-3 antibody binds to human LAG-3 with a K_{D} of 5×10⁻⁸ M or less, binds to human LAG-3 with a K_{D} of 1×10⁻⁸ M or less, binds to human LAG-3 with a K_{D} of 5×10⁻⁹ M or less, or binds to human LAG-3 with a K_{D} of between 1×10⁻⁸ M and Ix 10⁻¹⁰ M or less.

Administration of agonist anti-huCD40 antibodies described herein and antagonists, e.g., antagonist antibodies, to one or more second target antigens such as LAG-3 and/or CTLA-4 and/or PD-1 and/or PD-L1 can enhance the immune response to cancerous cells in the patient. Cancers whose growth may be inhibited using the antibodies of the instant disclosure include cancers typically responsive to immunotherapy. Representative examples of cancers for treatment with the combination therapy of the instant disclosure include those cancers specifically listed above in the discussion of monotherapy with agonist anti-huCD40 antibodies.

In certain embodiments, the combination of therapeutic antibodies discussed herein can be administered concurrently as a single composition in a pharmaceutically acceptable carrier, or concurrently as separate compositions with each antibody in a pharmaceutically acceptable carrier. In another embodiment, the combination of therapeutic antibodies can be administered sequentially. For example, an anti-CTLA-4 antibody and an agonist anti-huCD40 antibody can be administered sequentially, such as anti-CTLA-4 antibody being administered first and agonist anti-huCD40 antibody second, or agonist anti-huCD40 antibody being administered first and anti-CTLA-4 antibody second. Additionally or alternatively, an anti-PD-1 antibody and an agonist anti-huCD40 antibody can be administered sequentially, such as anti-PD-1 antibody being administered first and agonist anti-huCD40 antibody second, or agonist anti-huCD40 antibody being administered first and anti-PD-1 antibody second. Additionally or alternatively, an anti-PD-L1 antibody and an agonist anti-huCD40 antibody can be administered sequentially, such as anti-PD-L1 antibody being administered first and agonist anti-huCD40 antibody 15 second, or agonist anti-huCD40 antibody being administered first and anti-PD-L1 antibody second. Additionally or alternatively, an anti-LAG-3 antibody and an agonist anti-huCD40 antibody can be administered sequentially, such as anti-LAG-3 antibody being administered first and agonist anti-huCD40 antibody second, or agonist anti-huCD40 antibody being administered first and anti-LAG-3 antibody second.

Furthermore, if more than one dose of the combination therapy is administered sequentially, the order of the sequential administration can be reversed or kept in the same order at each time point of administration, sequential administrations can be combined with concurrent administrations, or any combination thereof. For example, the first administration of a combination anti-CTLA-4 antibody and agonist anti-huCD40 antibody can be concurrent, the second administration can be sequential with anti-CTLA-4 antibody first and agonist anti-huCD40 antibody second, and the third administration can be sequential with agonist anti-huCD40 antibody first and anti-CTLA-4 antibody second, etc. Additionally or alternatively, the first administration of a combination anti-PD-1 antibody and agonist anti-huCD40 antibody can be concurrent, the second administration can be sequential with anti-PD-1 antibody first and agonist anti-huCD40 antibody second, and the third administration can be sequential with agonist anti-huCD40 antibody first and anti-PD-1 antibody second, etc. Additionally or alternatively, the first administration of a combination anti-PD-L1 antibody and agonist anti-huCD40 antibody can be concurrent, the second administration can be sequential with anti-PD-L1 antibody first and agonist anti-huCD40 antibody second, and the third administration can be sequential with agonist anti-huCD40 antibody first and anti-PD-L1 antibody second, etc. Additionally or alternatively, the first administration of a combination anti-LAG-3 antibody and agonist anti-huCD40 antibody can be concurrent, the second administration can be sequential with anti-LAG-3 antibody first and agonist anti-huCD40 antibody second, and the third administration can be sequential with agonist anti-huCD40 antibody first and anti-LAG-3 antibody second, etc. Another representative dosing scheme can involve a first administration that is sequential with agonist anti-huCD40 first and anti-CTLA-4 antibody (and/or anti-PD-1 antibody and/or anti-PD-L1 antibody and/or anti-LAG-3 antibody) second, and subsequent administrations may be concurrent.

Optionally, an agonist anti-huCD40 as sole immunotherapeutic agent, or the combination of an agonist anti-huCD40 antibody and one or more additional immunotherapeutic antibodies (e.g., anti-CTLA-4 and/or anti-PD-1 and/or anti-PD-L1 and/or anti-LAG-3) can be further combined with an immunogenic agent, such as cancerous cells, purified tumor antigens (including recombinant proteins, peptides, and carbohydrate molecules), cells, and cells transfected with genes encoding immune stimulating cytokines (He et al. (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF (discussed further below). A CD40 agonist and one or more additional antibodies (e.g., CTLA-4 and/or PD-1 and/or PD-L1 and/or LAG-3 blockade) can also be further combined with standard cancer treatments. For example, a CD40 agonist and one or more additional antibodies (e.g., CTLA-4 and/or PD-1 and/or PD-L1 and/or LAG-3 blockade) can be effectively combined with chemotherapeutic regimes. In these instances, it is possible to reduce the dose of other chemotherapeutic reagent administered with the combination of the instant disclosure (Mokyr et al. (1998) Cancer Research 58: 5301-5304). An example of such a combination is a combination of CD40 agonist antibody with or without and an additional antibody, such as anti-CTLA-4 antibodies and/or anti-PD-1 antibodies and/or anti-PD-L1 antibodies and/or anti-LAG-3 antibodies) further in combination with decarbazine for the treatment of melanoma. Another example is a combination of agonist anti-huCD40 antibody with or without and anti-CTLA-4 antibodies and/or anti-PD-1 antibodies and/or anti-PD-L1 antibodies and/or LAG-3 antibodies further in combination with interleukin-2 (IL-2) for the treatment of melanoma. The scientific rationale behind the combined use of CD40 agonism and CTLA-4 and/or PD-1 and/or PD-L1 and/or LAG-3 blockade with chemotherapy is that cell death, which is a consequence of the cytotoxic action of most chemotherapeutic compounds, should result in increased levels of tumor antigen in the antigen presentation pathway. Other combination therapies that may result in synergy with a combined CD40 agonism with or without and CTLA-4 and/or PD-1 and/or PD-L1 and/or LAG-3 blockade through cell death include radiation, surgery, or hormone deprivation. Each of these protocols creates a source of tumor antigen in the host. Angiogenesis inhibitors can also be combined with a combined CD40 agonism and CTLA-4 and/or PD-1 and/or PD-L1 and/or LAG-3 blockade. Inhibition of angiogenesis leads to tumor cell death, which can be a source of tumor antigen fed into host antigen presentation pathways.

An agonist anti-huCD40 antibody as sole immunotherapeutic agent, or a combination of CD40 agonist and CTLA-4 and/or PD-1 and/or PD-L1 and/or LAG-3 blocking antibodies can also be used in combination with bispecific antibodies that target Fcα or Fcγ receptor-expressing effector cells to tumor cells. See, e.g., U.S. Pat. Nos. 5,922,845 and 5,837,243. Bispecific antibodies can be used to target two separate antigens. The T cell arm of these responses would be augmented by the use of a combined CD40 agonism and CTLA-4 and/or PD-1 and/or PD-L1 and/or LAG-3 blockade.

In another example, an agonistic anti-CD40 antibody as sole immunotherapeutic agent or a combination of an anti-CD40 antibody and additional immunostimulating agent, e.g., anti-CTLA-4 antibody and/or anti-PD-1 antibody and/or anti-PD-L1 antibody and/or LAG-3 agent, e.g., antibody, can be used in conjunction with an anti-neoplastic antibody, such as RITUXAN® (rituximab), HERCEPTIN® (trastuzumab), BEXXAR* (tositumomab), ZEVALIN® (ibritumomab), CAMPATH® (alemtuzumab), LYMPHOCIDE® (eprtuzumab), AVASTIN® (bevacizumab), and TARCEVA® (erlotinib), and the like. By way of example and not wishing to be bound by theory, treatment with an anti-cancer antibody or an anti-cancer antibody conjugated to a toxin can lead to cancer cell death (e.g., tumor cells) which would potentiate an immune response mediated by the immunostimulating agent, e.g., CD40, TIGIT, CTLA-4, PD-1, PD-L1 or LAG-3 agent, e.g., antibody. In an exemplary embodiment, a treatment of a hyperproliferative disease (e.g., a cancer tumor) can include an anti-cancer agent, e.g., antibody, in combination with an agonist anti-huCD40 antibody and optionally an additional immunostimulating agent, e.g., anti-CTLA-4 and/or anti-PD-1 and/or anti-PD-L1 and/or anti-LAG-3 agent, e.g., antibody, concurrently or sequentially or any combination thereof, which can potentiate an anti-tumor immune responses by the host.

Provided herein are methods for altering an adverse event associated with treatment of a hyperproliferative disease (e.g., cancer) with an immunostimulatory agent, comprising administering an agonist anti-huCD40 antibody with or without and a subtherapeutic dose of anti-CTLA-4 and/or anti-PD-1 and/or anti-PD-L1 and/or anti-LAG-3 agent, e.g., antibody, to a subject. For example, the methods described herein provide for a method of reducing the incidence of immunostimulatory therapeutic antibody-induced colitis or diarrhea by administering a non-absorbable steroid to the patient. As used herein, a “non-absorbable steroid” is a glucocorticoid that exhibits extensive first pass metabolism such that, following metabolism in the liver, the bioavailability of the steroid is low, i.e., less than about 20%. In one embodiment described herein, the non-absorbable steroid is budesonide. Budesonide is a locally-acting glucocorticosteroid, which is extensively metabolized, primarily by the liver, following oral administration. ENTOCORT EC® (Astra-Zeneca) is a pH- and time-dependent oral formulation of budesonide developed to optimize drug delivery to the ileum and throughout the colon. ENTOCORT EC© is approved in the U.S. for the treatment of mild to moderate Crohn's disease involving the ileum and/or ascending colon. The usual oral dosage of ENTOCORT EC® for the treatment of Crohn's disease is 6 to 9 mg/day. ENTOCORT EC® is released in the intestines before being absorbed and retained in the gut mucosa. Once it passes through the gut mucosa target tissue, ENTOCORT EC® is extensively metabolized by the cytochrome P450 system in the liver to metabolites with negligible glucocorticoid activity. Therefore, the bioavailability is low (about 10%). The low bioavailability of budesonide results in an improved therapeutic ratio compared to other glucocorticoids with less extensive first-pass metabolism. Budesonide results in fewer adverse effects, including less hypothalamic-pituitary suppression, than systemically-acting corticosteroids. However, chronic administration of ENTOCORT EC® can result in systemic glucocorticoid effects such as hypercorticism and adrenal suppression. See PDR 58^{th} ed. 2004; 608-610.

In still further embodiments, a CD40 agonist with or without CTLA-4 and/or PD-1 and/or PD-L1 and/or LAG-3 blockade (i.e., immunostimulatory therapeutic antibodies against CD40 and optionally anti-CTLA-4 and/or anti-PD-1 and/or anti-PD-L1 and/or anti-LAG-3 antibodies) in conjunction with a non-absorbable steroid can be further combined with a salicylate. Salicylates include 5-ASA agents such as, for example: sulfasalazine (AZULFIDINE®, Pharmacia & UpJohn); olsalazine (DIPENTUM®, Pharmacia & UpJohn); balsalazide (COLAZAL®, Salix Pharmaceuticals, Inc.); and mesalamine (ASACOL®, Procter & Gamble Pharmaceuticals; PENTASA®, Shire US; CANASA®, Axcan Scandipharm, Inc.; ROWASA®, Solvay).

In accordance with the methods described herein, a salicylate administered in combination with agonist anti-huCD40 antibody with or without anti-CTLA-4 and/or anti-PD-1 and/or anti-PD-L1 and/or LAG-3 antibodies and a non-absorbable steroid can includes any overlapping or sequential administration of the salicylate and the non-absorbable steroid for the purpose of decreasing the incidence of colitis induced by the immunostimulatory antibodies. Thus, for example, methods for reducing the incidence of colitis induced by the immunostimulatory antibodies described herein encompass administering a salicylate and a non-absorbable concurrently or sequentially (e.g., a salicylate is administered 6 hours after a non-absorbable steroid), or any combination thereof. Further, a salicylate and a non-absorbable steroid can be administered by the same route (e.g., both are administered orally) or by different routes (e.g., a salicylate is administered orally and a non-absorbable steroid is administered rectally), which may differ from the route(s) used to administer the anti-huCD40 and anti-CTLA-4 and/or anti-PD-1 and/or anti-PD-L1 and/or anti-LAG-3 antibodies.

The agonist anti-huCD40 antibodies and combination antibody therapies described herein may also be used in conjunction with other well known therapies that are selected for their particular usefulness against the indication being treated (e.g., cancer). Combinations of the agonist anti-huCD40 antibodies described herein may be used sequentially with known pharmaceutically acceptable agent(s).

For example, the agonist anti-huCD40 antibodies and combination antibody therapies described herein can be used in combination (e.g., simultaneously or separately) with an additional treatment, such as irradiation, chemotherapy (e.g., using camptothecin (CPT-11), 5-fluorouracil (5-FU), cisplatin, doxorubicin, irinotecan, paclitaxel, gemcitabine, cisplatin, paclitaxel, carboplatin-paclitaxel (Taxol), doxorubicin, 5-fu, or camptothecin+apo2l/TRAIL (a 6×combo)), one or more proteasome inhibitors (e.g., bortezomib or MG132), one or more Bc1-2 inhibitors (e.g., BH3I-2′ (bcl-xl inhibitor), indoleamine dioxygenase-1 (IDO1) inhibitor (e.g., INCB24360), AT-101 (R-(−)-gossypol derivative), ABT-263 (small molecule), GX-15-070 (obatoclax), or MCL-1 (myeloid leukemia cell differentiation protein-1) antagonists), iAP (inhibitor of apoptosis protein) antagonists (e.g., smac7, smac4, small molecule smac mimetic, synthetic smac peptides (see Fulda et al., Nat Med 2002; 8:808-15), ISIS23722 (LY2181308), or AEG-35156 (GEM-640)), HDAC (histone deacetylase) inhibitors, anti-CD20 antibodies (e.g., rituximab), angiogenesis inhibitors (e.g., bevacizumab), anti-angiogenic agents targeting VEGF and VEGFR (e.g., AVASTIN®), synthetic triterpenoids (see Hyer et al., Cancer Research 2005; 65:4799-808), c-FLIP (cellular FLICE-inhibitory protein) modulators (e.g., natural and synthetic ligands of PPARy (peroxisome proliferator-activated receptor γ), 5809354 or 5569100), kinase inhibitors (e.g., Sorafenib), trastuzumab, cetuximab, Temsirolimus, mTOR inhibitors such as rapamycin and temsirolimus, Bortezomib, JAK2 inhibitors, HSP90 inhibitors, P13K-AKT inhibitors, Lenalildomide, GSK30 inhibitors, IAP inhibitors and/or genotoxic drugs.

The agonist anti-huCD40 antibodies and combination antibody therapies described herein can further be used in combination with one or more anti-proliferative cytotoxic agents. Classes of compounds that may be used as anti-proliferative cytotoxic agents include, but are not limited to, the following: Alkylating agents (including, without limitation, nitrogen mustards, ethylenimine derivatives, alkyl sulfonates, nitrosoureas and triazenes): Uracil mustard, Chlormethine, Cyclophosphamide (CYTOXAN™) fosfamide, Melphalan, Chlorambucil, Pipobroman, Triethylenemelamine, Triethylenethiophosphoramine, Busulfan, Carmustine, Lomustine, Streptozocin, Dacarbazine, and Temozolomide.

Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Suitable anti-proliferative agents for combining with agonist anti-huCD40 antibodies, without limitation, taxanes, paclitaxel (paclitaxel is commercially available as TAXOL™), docetaxel, discodermolide (DDM), dictyostatin (DCT), Peloruside A, epothilones, epothilone A, epothilone B, epothilone C, epothilone D, epothilone E, epothilone F, furanoepothilone D, desoxyepothilone Bl, [17]-dehydrodesoxyepothilone B, [18]dehydrodesoxyepothilones B, C12,13-cyclopropyl-epothilone A, C6-C8 bridged epothilone A, trans-9,10-dehydroepothilone D, cis-9,10-dehydroepothilone D, 16-desmethylepothilone B, epothilone B10, discoderomolide, patupilone (EPO-906), KOS-862, KOS-1584, ZK-EPO, ABJ-789, XAA296A (Discodermolide), TZT-1027 (soblidotin), ILX-651 (tasidotin hydrochloride), Halichondrin B, Eribulin mesylate (E-7389), Hemiasterlin (HTI-286), E-7974, Cyrptophycins, LY-355703, Maytansinoid immunoconjugates (DM-1), MKC-1, ABT-751, T1-38067, T-900607, SB-715992 (ispinesib), SB-743921, MK-0731, STA-5312, eleutherobin, 17beta-acetoxy-2-ethoxy-6-oxo-B-homo-estra-1,3,5(10)-trien-3-ol, cyclostreptin, isolaulimalide, laulimalide, 4-epi-7-dehydroxy-14,16-didemethyl-(+)-discodermolides, and cryptothilone 1, in addition to other microtubuline stabilizing agents known in the art.

In cases where it is desirable to render aberrantly proliferative cells quiescent in conjunction with or prior to treatment with agonist anti-huCD40 antibodies described herein, hormones and steroids (including synthetic analogs), such as 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, ZOLADEX™, can also be administered to the patient. When employing the methods or compositions described herein, other agents used in the modulation of tumor growth or metastasis in a clinical setting, such as antimimetics, can also be administered as desired.

Methods for the safe and effective administration of chemotherapeutic agents are known to those skilled in the art. In addition, their administration is described in the standard literature. For example, the administration of many of the chemotherapeutic agents is described in the Physicians' Desk Reference (PDR), e.g., 1996 edition (Medical Economics Company, Montvale, N.J. 07645-1742, USA); the disclosure of which is incorporated herein by reference thereto.

The chemotherapeutic agent(s) and/or radiation therapy can be administered according to therapeutic protocols well known in the art. It will be apparent to those skilled in the art that the administration of the chemotherapeutic agent(s) and/or radiation therapy can be varied depending on the disease being treated and the known effects of the chemotherapeutic agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

### XI. Characterization of Specific Agonist Anti-CD40 Antibodies of the Present Invention

Agonist anti-CD40 antibodies of the present invention were obtained as described in Example 1. Variable domains and CDR sequence regions of exemplary antibodies of the present invention are provided in the Sequence Listing, and are summarized at Table 2. Variable domain and CDR region numbering is the same for all antibodies derived from the same original clone, i.e. the humanized variants provided herein do not include any insertions or deletions.

TABLE 2
Antibody Variable Domains and CDRs

| | | Variable | | | |
|---|---|---|---|---|---|
| Clone | Chain | Domain | CDR1 | CDR2 | CDR3 |
| 12D6 | Heavy chain | 1-119 | 31-35 | 50-66 | 99-108 |
| 12D6 | Light chain | 1-112 | 24-39 | 55-61 | 94-102 |
| 5F11 | Heavy chain | 1-117 | 31-35 | 50-66 | 99-106 |
| 5F11 | Light chain | 1-111 | 24-38 | 54-60 | 93-101 |
| 8E8 | Heavy chain | 1-122 | 31-35 | 50-66 | 99-111 |
| 8E8 | Light chain | 1-112 | 24-39 | 55-61 | 94-102 |
| 5G7 | Heavy chain | 1-113 | 31-35 | 50-66 | 99-102 |
| 5G7 | Light chain | 1-107 | 24-34 | 50-56 | 89-97 |
| 19G3 | Heavy chain | 1-112 | 31-35 | 50-66 | 99-101 |
| 19G3 | Light chain | 1-112 | 24-39 | 55-61 | 94-102 |

The invention also provides anti-huCD40 antibodies related to those disclosed herein by sequence by being derived from the same murine germline sequences, specifically the V and J region gene segments. The murine germline sequences for each of the antibodies disclosed herein are provided at Table 3.

TABLE 3
Mouse Germline Sequences for anti-huCD40 mAbs

| Clone | Chain | V region | J region |
|---|---|---|---|
| 12D6 | Heavy chain | VH1-39_01 | IGHJ4 |
| 12D6 | Light chain | VK1-110_01 | IGKJ1 |
| 5F11 | Heavy chain | VH1-4_02 | IGHJ3 |
| 5F11 | Light chain | VK3-5_01 | IGKJ5 |
| 8E8 | Heavy chain | VH1-80_01 | IGHJ2 |
| 8E8 | Light chain | VK1-110_01 | IGKJ2 |
| 5G7 | Heavy chain | VH1-18_01 | IGHJ4 |
| 5G7 | Light chain | VK10-96_01 | IGKJ2 |
| 19G3 | Heavy chain | VH5-9-4_01 | IGHJ3 |
| 19G3 | Light chain | VK1-117_01 | IGKJ2 |

The invention further provides humanized anti-huCD40 antibodies derived from the murine parental antibodies disclosed herein by replacement of murine framework sequences with human framework sequences, with or without additional mutations (“back mutations”) to restore antigen (human CD40) affinity that would otherwise be lost in the humanization or to remove sequence liabilities. See Example 3.

The invention also provides antibody constructs comprising the novel variable domain sequences disclosed herein and constant domains with modified Fc regions having enhanced affinity for FcγRIIb as compared with their affinity for other Fc receptors, i.e. activating receptors. Such agonistic anti-huCD40 antibodies with enhanced FcγRIIb-specificity are expected to exhibit superior efficacy in treatment of cancer and chronic infection. Li & Ravetch (2011) Science 333:1030; White et al. (2011) J. Immunol. 187:1754. Without intending to be limited by theory, such FcγRIIb-specific agonistic anti-CD40 mAbs may exhibit enhanced adjuvant effects by increasing the maturation of dendritic cells promoting expansion and activation of cytotoxic CD8⁺ T cells, leading to enhanced anti-tumor response. Id. Without intending to be limited by theory, FcR-mediated signal enhancement of agonist CD40 antibodies due to increased receptor clustering, or “cross-linking,” of the present invention may be a major contributor to therapeutic efficacy. Cross-linking of CD40 agonist antibodies by FcR engagement by the Fc portion of the antibody may increase signal strength and thereby enhance activation of cells.

The relative binding affinity of antibodies for activating (A) versus inhibitory (I) Fc receptors can be expressed as the “A/I” ratio, and is typically a function of the structure of the Fc region of an IgG antibody. See WO 2012/087928. Antibodies having enhanced specificity for binding to inhibitory receptor FcγRIIb have lower A/I ratios. Preferred antibodies for the agonistic anti-huCD40 antibodies of the present invention have, for example, A/I ratios of less than 5, 4, 3, 2, 1, 0.5, 0.3, 0.1, 0.05, 0.03 or 0.01.

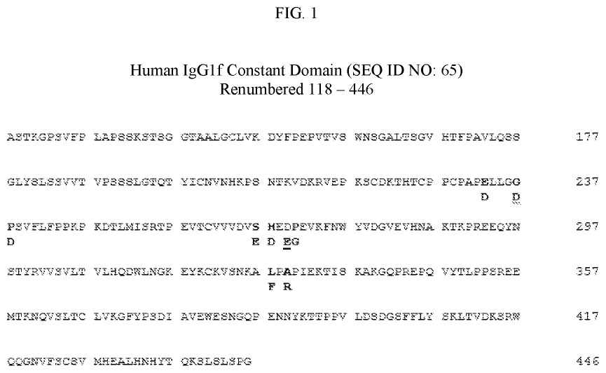

Human IgG1 constant domains comprising mutations to enhance FcγRIIb specificity are also provided in the Sequence Listing, and are summarized at Table 4 and illustrated at FIG. 1. Sequence variants are defined with reference to human IgG1f constant domain sequence provided at SEQ ID NO: 65. The nomenclature regarding positions (numbering) of mutations in the Fc region is according to the EU index as in Kabat et al. (1981) Sequences of Proteins of Immunological Interest, 5th Ed. Public Health Service, National Institutes of Health, Bethesda, Md.), which facilitates comparison of Fc sequences at equivalent positions in antibodies with differing variable domain lengths. See also Edelman et al. (1969) Proc. Nat'l Acad. Sci. USA 63:78; WO 2012/130831 (using the same numbering system). It does not match the sequence numbering in the Sequence Listing. FIG. 1 provides a graphical representation of the Fc sequence variants of Table 4, from which one of skill in the art could readily recognize the corresponding positions in the antibody sequences disclosed herein. SE and SELF variants are described at Chu et al. (2008) Mol. Immunol. 45:3926. P238D, V4, V7, V8, V9, V11 and V12 variants are described at Mimoto et al. (2013) Protein Engineering Design & Selection 26:589 (e.g. at table 1 therein).

TABLE 4
Fc Sequence Variants

| Variant | SEQ | | | | | | | | | |
|---|---|---|---|---|---|---|---|---|---|---|
| Name | ID: | Sequence Variants | | | | | | | | |
| IgG1f | 65 | | | | | | | | | |
| SE | 66 | | | | S267E | | | | | |
| SELF | 67 | | | | S267E | | | | L328F | |
| P238D | 68 | | | P238D | | | | | | |
| V4 | 69 | | | P238D | | | | P271G | | |
| V4 - | 70 | | | P238D | | | D270E | P271G | | |
| D270E | | | | | | | | | | |
| V7 | 71 | E233D | | P238D | | | | P271G | | A330R |
| V8 | 72 | | G237D | P238D | | H268D | | P271G | | |
| V9 | 73 | | G237D | P238D | | | | P271G | | A330R |
| V9- | 74 | | G237D | P238D | | | D270E | P271G | | A330R |
| D270E | | | | | | | | | | |
| V11 | 75 | | G237D | P238D | | H268D | | P271G | | A330R |
| V12 | 76 | E233D | G237D | P238D | | H268D | | P271G | | A330R |

Additional Fc sequence variants with enhanced affinity for FcγRIIb are disclosed at Yu et al. (2013) J Am. Chem. Soc. 135:9723 (and WO 2014/184545), including V262E and V264E, e.g. for use in combination with S267E and L328F.

Additional variants with D270E mutations were produced to reduce the D270 isomerization that occurred in the V4 and V9 Fc sequence variants, which otherwise exhibited enhanced isomerization rates in accelerated degradation studies suggesting ˜12-16% isomerization after two years at 4° C. in PBS, compared with ˜5-7% for other variants. See Table 5 (providing data from a single experiment but replicate experiments showed comparable values). Substitution of aspartic acid at position 270 with glutamic acid eliminated the possibility of such DG isomerization, resulting in antibodies that are more chemically stable and antibody preparations that are more homogenous. Other D270 substitutions of V9, such as D270A, D270Q, D270S and D270T, effectively eliminated binding to FcγRIIa and FcγRIIb receptors (K_{D} was greater than 5 μM). Although the D270E mutation reduced binding to FcγRIIb receptor by an order of magnitude, the V9-D270E variant maintained a favorable bias in affinity for FcγRIIb receptor compared with FcγRIIa, and acceptable absolute affinity for FcγRIIb. See Example 8.

TABLE 5
Fc Sequence Variant Affinity for Fcγ Receptors (K_{D} in nM)

| Designation | FcγRIIa-H131 | FcγRIIa-R131 | FcγRIIb |
|---|---|---|---|
| IgG1f | 530 | 850 | 3900 |
| SE | 520 | 22 | 98 |
| SELF | 1100 | 2 | 11 |
| P238D | >5000 | >5000 | 950 |
| V4 | >5000 | 1900 | 150 |
| V4 - D270E | >5000 | >5000 | 1800 |
| V7 | >5000 | 1600 | 84 |
| V8 | >5000 | 1400 | 93 |
| V9 | >5000 | 420 | 15 |
| V9 - D270A, Q, S, T | >5000 | >5000 | >5000 |
| V9 - D270E | >5000 | >5000 | 150 |
| V11 | — | 450 | 15 |
| V12 | >5000 | 490 | 21 |

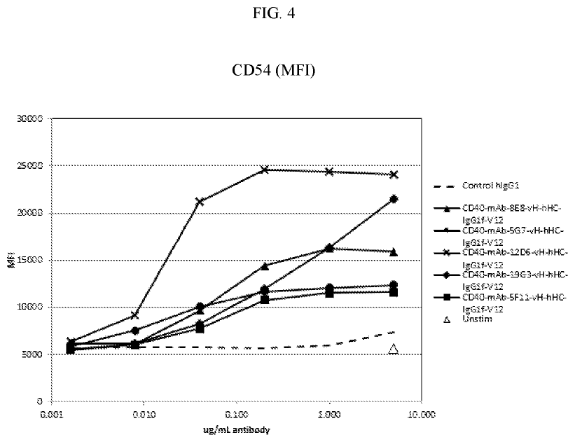

Agonist activity of various anti-CD40 antibodies of the present invention was measured. See Example 7, and FIGS. 3A, 3B and 4. Agonist activity was found to depend on both the variable domain sequences (mAb clone number), which determine antigen (human CD40) binding, and the sequence of the Fc region, which determines Fc receptor (FcγRIIb) binding.

The present disclosure is further illustrated by the following examples, which should not be construed as further limiting. The contents of all figures and all references, Genbank sequences, patents and published patent applications cited throughout this application are expressly incorporated herein by reference.

### EXAMPLES

### Example 1

Generation of Mouse Monoclonal Antibodies against Human CD40

Murine anti-human CD40 monoclonal antibodies were generated using wild type Balb/c mice (Charles Rivers Labs) that express mouse antibody genes, as follows.

### Antigen

A huCD40-muFc soluble recombinant protein was used as the antigen for immunizations. The soluble fusion protein has a MW of 91.6 kD and is composed of the extracellular portion of huCD40 linked to a mouse IgG2b Fc at its C-terminus. This fusion protein is referred to herein as “huCD40-muFc fusion protein”. The fusion protein was generated by standard recombinant DNA methods and expressed in transfected CHO cells, which secreted the soluble fusion protein into the culture supernatant. The CHO host cells used for transfection were obtained from Invitrogen (Cat #11619-012). The secreted soluble fusion protein was purified for use as immunogen.

### Immunization of Mice

To generate mouse monoclonal antibodies to human CD40, Balb/c mice were immunized with purified huCD40-muFc fusion protein. The mice were approximately 2-4 months of age upon the first infusion of antigen. Purified recombinant huCD40-muFc antigen preparation (10 μg purified from transfected mammalian cells expressing the fusion protein) was used to immunize the mice using two immunization protocols (A and B). Protocol A consisted of four weekly footpad (FP) immunizations and protocol B of seven weekly subcutaneous (SC)/intraperitoneal (IP)/Hock immunizations. In both cases, the immunogen was mixed 1:1 with RIBI adjuvant (Sigma Cat #M6536).

All mice were bled one week after the fourth immunizations to assess antigen specific titers. Final bleeds were also obtained from each mouse at time of sacrifice. The immune response was monitored by retro orbital bleeds. The plasma was screened by ELISA analysis using the recombinant protein used for immunizations. Mice from protocol A received two final boosts intravenously (IV) and in the footpads (FP) with soluble antigen on days −2 and −3 before fusion. Mice from protocol B received two final boosts intravenously as well as IP and in the hock with the soluble antigen on days −2 and −3 before fusion.

All four mice from protocol A (IDs 291763, 291764, 291765, 291766) were sacrificed. Lymph node and spleen cells were extracted and mixed for fusion (fusions 3582 and 3583). Only two mice from protocol B (IDs 294286 and 294288) were sacrificed. Spleen and lymph nodes from each mouse were mixed and fused (fusions 3716 and 3717).

### Generation of Hybridomas Producing Monoclonal Antibodies to Human CD40

Mouse splenocytes and lymph nodes isolated from high titer Balb/c mice were fused with a mouse myeloma fusion partner using the electric field based electrofusion Hybrimune instrument and a large 0.9 ml fusion chamber (BTX Harvard Apparatus, Inc., Holliston, MA). Single cell suspensions of lymphocytes from immunized mice were fused to an equal number of P3X63 Ag8.6.53 (ATCC CRL 1580) non-secreting mouse myeloma cells. Resulting cells were plated at 2.0×10⁴ cells/well in flat bottom microtiter plates in selective ClonaCell-HY Medium E (catalog #03805; STEMCELL Technologies Inc., Vancouver BC, Canada) with addition of Aminopterin to select for hybridomas. After about 7 days, the culture medium was replaced with Medium E (without aminopterin).

After 10 to 12 days, individual wells were screened for the presence of mouse IgG/mouse kappa light chain antibodies using a homogenous HTRF assay. In this assay, supernatants from 96 well fusion plates were mixed with custom labeled Terbium-Cryptate goat anti-mouse IgG (Fcγ specific) (Cisbio US Inc. Bedford, MA) and goat anti-mouse IgG Fab′2 labeled with AlexaFluor 647 (Jackson ImmunoResearch; Catalog #109-605-098). Incubation was for 1 hour. The plates were then read on a Rubystar reader. Hybridoma cells from wells positive for mouse IgG/mouse kappa light chain antibodies were then screened either by FACS using CHO cells transfected with human CD40 and CHO untransfected cells as control (fusions 3582/3583) or by ELISA using recombinant protein followed by FACS on Daudi B cells and Jurkat T cells as negative control (fusions 3716/3717). FACS positive parental lines were transferred to 24-well plates. A few days later, cell supernatants from individual wells were rescreened by FACS to confirm IgG specificity to human CD40.

A panel of antigen specific hybridomas were cloned by serial dilution and rescreened by FACS using either CHO transfectants or Daudi cells. Nineteen antibodies from fusions 3582/3583 and twenty antibodies from fusions 3716/3717 were purified and tested for functional activity. From this panel of antibodies, five strong agonists were selected for second subcloning. These five antibodies, namely 1802.3582.19G3.F10.E1, 1802.3583.5G7.F12.G3, 1802.3583.8E8.C10.G2, 1802.3716.12D6.B1.E3, and 1802.3717.5F11.A11.E7 were sequenced and further analyzed.

### Example 2

Generation of Fully Human anti-huCD40 Antibodies

Fully human anti-huCD40 monoclonal antibodies that bind to the same epitope and/or cross-block the binding of the humanized anti-CD40 antibodies disclosed herein may find use in methods of the present invention. Such antibodies may be generated using transgenic mice that express human antibody genes, as follows.

### Antigen

A huCD40 soluble recombinant protein is used as the antigen for immunization. The soluble fusion protein is composed of the extracellular portion of huCD40 linked to a mouse IgG2a Fe at its C-terminus. This fusion protein is referred to herein as “huCD40-muFc fusion protein.” The fusion protein is generated by standard recombinant DNA methods and expressed in transfected CHO cells, which secrete the soluble fusion protein into the culture supernatant. The CHO host cells used for transfection are obtained from Invitrogen (Cat #11619-012). The secreted soluble fusion protein is purified for use as immunogen. The sequence of full length human CD40 including signal sequence is provided at SEQ ID NO: 1.

### Transgenic Mice

Fully human monoclonal antibodies to human CD40 are prepared using mice from the CMD++; JKD++; KCo5(9272)+\hat{ }; SC20+ genotype (hereafter called KM® mice). Individual transgene designations are in parentheses, followed by line numbers for randomly integrated transgenes. The symbols++ and +indicate homozygous or hemizygous; however, because the mice are routinely screened using a PCR-based assay that does not allow us to distinguish between heterozygosity and homozygosity for the randomly integrated human Ig transgenes, a + designation may be given to mice that are actually homozygous for these elements. In this strain, the endogenous mouse kappa light chain gene has been homozygously disrupted as described in Chen et al. (1993) EMBO J. 12:811-820 and the endogenous mouse heavy chain gene has been homozygously disrupted as described in example 1 of WO 2001/09187. Furthermore, this mouse strain carries a human kappa light chain transgene, KCo5, as described in Fishwild et al. (1996) Nature Biotechnology 14:845-851, a yeast artificial chromosome (YAC) carrying most of the human kappa light chain locus, as described in WO 2000/026373.

### Immunization of Mice

To generate fully human monoclonal antibodies to human CD40, KM® mice are immunized with purified huCD40-muFc fusion protein. General immunization schemes are described in Lonberg et al. (1994) Nature 368(6474): 856-859; Fishwild, D. et al. (1996) Nature Biotechnology 14: 845-851 and WO 98/24884. The mice are approximately 4 months of age upon the first infusion of antigen. Either purified recombinant huCD40-muFc antigen preparation (10 μg purified from transfected mammalian cells expressing the fusion protein) or 300-19 cells transfected with human CD40 are used to immunize the mice intraperitoneally and subcutaneously. The immunogens are mixed 1:1 with RIBI adjuvant (Sigma Cat #M6536).

The mice are immunized five times at 5-7 day intervals. The first and second immunizations are performed with the recombinant protein. The third immunization is with the cells, the fourth immunization with the protein and the fifth immunization with the cells. Mice are bled one week after the last immunizations to assess antigen specific titers. The immune response is monitored by retro orbital bleeds. The plasma is screened by FACS analysis using the transfected 300-19 cells, and mice with highest titers for anti-human CD40 human IgG are used for fusions. Mice receive a final boost by intravenous (IV) and intraperitoneal (IP) injection of soluble antigen two days and transfected cells three days before sacrifice and removal of the spleen.

### Generation of Hybridomas Producing Human Monoclonal Antibodies to Human CD40

Mouse splenocytes isolated from high titer KM® mice and a mouse myeloma fusion partner are fused with an electric field based electrofusion using a Cyto Pulse large chamber cell fusion electroporator (Cyto Pulse Sciences, Inc., Glen Burnie, MD). Single cell suspensions of splenic lymphocytes from immunized mice are fused to an equal number of P3X63 Ag8.6.53 (ATCC CRL 1580) non-secreting mouse myeloma cells (fusion number: 2541). Resulting cells are plated at 2.0×10⁴ cells/well in flat bottom microtiter plates in selective DMEM medium containing high glucose (Cellgro #10-013-CM) and 10% fetal calf serum (Hyclone #SH30071.03), and supplemented with beta-mercaptoethanol (1000×, Gibco #21985-023), 7 mM HEPES (Cellgro 25-060-Cl), additional 2 mM L-glutamine (Cellgro 25-005-Cl), HAT (50×, Sigma #H-0262), 5% Hybridoma Cloning Factor (BioVeris #210001), 10% P388DI (ATCC #CRL TIB-63) conditioned medium and Penicillin-Streptomycin (100×, Cellgro #30-002-Cl). After about 7 days, some of the medium containing HAT is replaced with medium containing HT (Cellgro #25-047-Cl).

After 10 to 12 days, individual wells are screened for the presence of human IgG/human kappa light chain antibodies using a homogenous HTRF assay. In this assay, supernatants from 96 well fusion plates are mixed with Europium-cryptate labeled goat anti-human IgG (Fc fragment specific), biotinylated goat anti-human kappa light chain (Bethyl #A80-115B), streptavidin-XLent and incubated for 1 hour. The plates are then read on a RUBYstar reader.

Hybridoma cells from wells positive for human IgG/human kappa light chain or human IgG/human lambda light chain antibodies are then screened by FACS using 300-19 cells transfected with human CD40 and 300-19 untransfected cells as control. FACS positive parental lines are transferred to 24-well plates. A few days later, cell supernatants from individual wells are rescreened by FACS to confirm IgG specificity to human CD40.

The hybridomas are cloned by serial dilution and re-screened by FACS.

### Example 3

Humanization of anti-huCD40 Antibodies

Parental (murine) antibodies of the present invention were humanized for potential use as human therapeutics. The closest matching human germline region sequence was selected for each mouse variable domain, and these human framework regions were used to replace murine frameworks in the “CDR graft” versions of the antibodies. Additional amino acid substitutions, such as framework back-mutations, were then made as necessary to restore the binding affinity of the humanized antibody. Further amino acid substitutions were made to eliminate sequence liabilities. The sequence relationships between the various forms of the antibodies of the present invention are provided at Table 6. The binding of various agonistic anti-huCD40 antibodies to soluble human CD40 was determined by Biolayer Interferometry (BLI) analysis using a Fortebio Octet RED (Rapid system—Extended Detection) label-free interaction analysis instrument. All studies were performed in 10 mM NaₓPO₄, 130 mM NaCl, 0.05% Surfactant P20 (pH 7.1) at 25° C. Briefly, anti-huCD40 antibody supernatants (diluted to 10 μg/ml or captured undiluted if supernatant concentration was less than 10 μg/ml) were captured on protein A coated biosensors (Pall Fortebio #18-5010) using a loading time of 90 s and shake speed of 1000 rpm. Supernatants were first screened for binding to 1 μM recombinant human CD40 monomer (huCD4O-monomer) using 180 s association and dissociation times, at 1000 rpm, with two 15 s conditioning steps using 10 mM glycine pH 1.5 in between binding cycles. All supernatants that demonstrated a binding signal in the 1 μM huCD40-monomer experiment were then tested for binding to seven different concentrations of huCD40-monomer in a three-fold dilution series, where the highest concentration was either 10 μM huCD40-monomer or 1 μM huCD40-monomer depending on the strength of the binding signal in the 1 μM screening experiment. Results for selected antibodies and sequence variants thereof are shown in Table 6.

TABLE 6
Binding Affinity of anti-CD40 Antibodies

| Antibody | Type | V_{H} | V_{L} | K_{D} (nM) |
|---|---|---|---|---|
| 12D6 | parental | mouse | mouse | 3.6 |
| 12D6-03 | humanized | VH1-18 CDR graft | VKII O11 CDR graft | 4.8 |
| 12D6-22 | humanized | VH1-18 CDR graft | VKII O11 G29A | 3.6 |
| 12D6-23 | humanized | VH1-18 M100bL | VKII O11 CDR graft | 4.3 |
| 12D6-24 | humanized | VH1-18 M100bL | VKII O11 G29A | 5.6 |
| 5F11 | parental | mouse | mouse | 4.7 |
| 5F11-03 | humanized | VH1-e CDR graft | VKIV B3 CDR graft | >5000 |
| 5F11-17 | humanized | VH1-e G27Y S30T | VKIV B3 CDR graft | 8.4 |
| | | V37I R38K A40R | | |
| | | M48I R66K V67T | | |
| | | I69L | | |
| 5F11-23 | humanized | VH1-e G27Y S30T | VKIV B3 M4L N22S | 4.9 |
| | | V37I R38K A40R | | |
| | | M48I R66K V67T | | |
| | | I69L | | |
| 5F11-45 | humanized | VH1-e G27Y S30T | VKIV B3 M4L | 12.3* |
| | | R66K V67T I69L | | |
| 19G3 | parental | mouse | mouse | 870 |
| 19G3-03 | humanized | VH3-21 CDR graft | VKII O11 CDR graft | no |
| | | | | binding |
| 19G3-11 | humanized | VH3-21 S94R | VKII O11 CDR graft | 850 |
| 19G3-22 | humanized | VH3-21 S94R | VKII O11 N28Q | 99 |
| 19G3-23 | humanized | VH3-21 S94R | VKII O11 G29A | 1300 |
| 5G7 | parental | mouse | mouse | 89 |
| 5G7-03 | humanized | VH1-18 CDR graft | VKI A20 CDR graft | 340 |
| 5G7-22 | humanized | VH1-18 M69L A93V | VKI A20 CDR graft | 50 |
| 5G7-25 | humanized | VH1-18 M69L T71V | VKI A20 CDR graft | 69 |
| | | A93V G55A | | |
| 5G7-28 | humanized | VH1-18 M69L T71V | VKI A20 CDR graft | 230 |
| | | A93V G55A M96L | | |
| 8E8 | parental | mouse | mouse | 85 |
| 8E8-05 | humanized | VH1-e CDR graft | VKII A19 CDR graft | no |
| | | | | binding |
| 8E8-56 | humanized | VH1-e L4F G27Y | VKII A19 CDR graft | 82 |
| | | S30T R66K V67A | | |
| | | T68L I69L | | |
| 8E8-62 | humanized | VH1-e L4F G27Y | VKII A19 CDR graft | 31 |
| | | S30T R66K V67A | | |
| | | T68L I69L D99E | | |
| 8E8-66 | humanized | VH1-e L4F G27Y | VKII A19 G29A | 82 |
| | | S30T R66K V67A | | |
| | | T68L I69L | | |
| 8E8-67 | humanized | VH1-e L4F G27Y | VKII A19 G29A | 28 |
| | | S30T R66K V67A | | |
| | | T68L I69L D99E | | |
| 8E8-70 | humanized | VH1-e L4F G27Y | VKII A19 CDR graft | low |
| | | S30T R66K V67A | | signal |
| | | T68L I69L D99E G55S | | |
| 8E8-71 | humanized | VH1-e L4F G27Y | VKII A19 G29A | low |
| | | S30T R66K V67A | | signal |
| | | T68L I69L D99E G55S | | |

*= for comparison purposes, the parental 5F11 exhibited a K_{D} of 8.2 nM (rather than 4.7 nM) when run in the same assay in parallel with the 5F11-45 antibody.

For humanized antibodies, V_{H} and V_{L} columns provide the human variable domain germlines on which framework regions are based. “CDR graft” refers to variable domains comprising unmodified parental (mouse) CDRs grafted directly onto the recited human framework sequences. Residue numbering for all sequence variants in Table 6 is according to Kabat, and thus the positions of sequence changes in Table 6 does not match the residue numbering of the antibody sequences in the Sequence Listing. Underlining is used to indicate sequence modifications that are intended to correct potential sequence liabilities, i.e. residues that are subject to chemical modification and potential degradation, leading to product heterogeneity. Other sequence modifications are intended to restore affinity, typically a framework back mutation (reverting from the human germline framework sequence to the original murine framework residue). The M69L modification in the heavy chain of antibody 5G7 is both a framework back mutation and a liability correction.

### Example 4

### Anti-CD40 Antibody Epitope Binning Experiments

Epitope binning experiments may be conducted to determine which anti-human CD40 antibodies compete with which others for binding to huCD40, and thus bind to similar epitopes. Pairwise competition between anti-huCD40 antibodies is determined as follows, in which a reference antibody is bound to the surface of a sensor chip, a test antibody is pre-incubated with a huCD40 polypeptide construct in a mixture, and the pre-incubated mixture is flowed over the sensor chip to determine the degree to which the test antibody interferes with binding of the huCD40 polypeptide construct to the reference antibody on the chip surface. Such competition experiments may be performed using a BIACORE® SPR instrument. Briefly, a reference anti-huCD40 antibody is immobilized onto Sensor Chip CM5 chip (Series S, GE Healthcare CAT #BR-1005-30) surfaces, flowcell2, flowcell3 & flowcell4 (5000 RUs), and flowcell1 is used as a negative control. A test antibody is diluted to 120 μg/mL (2×) at starting concentration. A series of dilutions of the test antibody is made by diluting 1:3 concentration of antibody with buffer for seven different concentrations and a control sample (with 0 μg/ml) to obtain a titration curve. Each antibody concentration series is divided into two halves. In the first half of the concentration series, 40 nM (2×) human CD40 antigen (e.g. huCD40/Fc) is added to make the final concentration series (60 μg/ml-0.0 μg/ml) and 20 nM of final antigen concentration in each well. In the second half of the concentration series, in place of antigen, buffer is added to have the antibody diluted to the same concentration, and this half is treated as the blank. Complexes of the test anti-CD40 antibodies and huCD40/Fc are incubated for 2 hours. 40 μL complexes are injected on the reference antibody-coated surface at a 30 μL/min. A BIACORE® T200 surface plasmon resonance instrument is used and the running buffer is HBE-EP, GE Healthcare CAT #BR-1001-88, filtered, degassed, 0.01M HEPES, pH7.4, 0.15 NaCl, 3 mM EDTA, 0.005% Surfactant P20. The surface is regenerated with 25 mM NaOH (order code: BR-1003-58, GE Healthcare) at 100 μL/min for 5 seconds. The data are analyzed using Microsoft Excel where the concentration of test antibodies is plotted against the corresponding response unit to obtain titration curves.

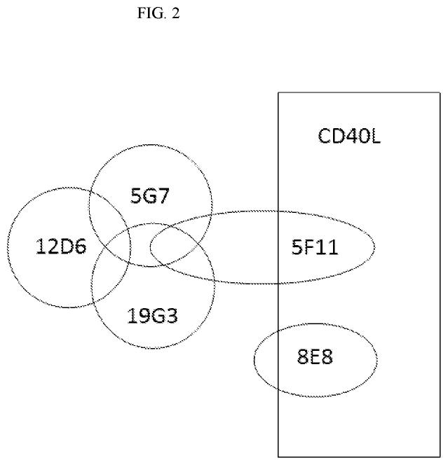

Results of such epitope binning experiments for the anti-CD40 antibodies of the present invention are provided at FIG. 2. Antibodies 5F11 and 8E8 block ligand (CD40L) binding. The five anti-CD40 antibodies of the present invention fall into three epitope groups-12D6/5G7/19G3, 5F11/5G7/19G3, and 8E8.

### Example 5

### Epitope Mapping by HDX

The epitopes for anti-huCD40 antibodies 12D6, 5G7, 19G3 and 5F11 of the present invention were determined by hydrogen/deuterium exchange mass spectrometry (HDX-MS). HDX-MS probes protein conformation and conformational dynamics in solution by monitoring the rate and extent of deuterium exchange of backbone amide hydrogen atoms. Huang & Chen (2014) Anal. Bioanalytical Chem. 406:6541; Wei et al. (2014) Drug Disc. Today 19:95. The level of HDX depends on the solvent accessibility of backbone amide hydrogen atoms and the protein hydrogen bonds. The mass increase of the protein upon HDX can be precisely measured by MS. When this technique is paired with enzymatic digestion, structure features at the peptide level can be resolved, enabling differentiation of surface exposed peptides from those folded inside, or from those sequestered at the interface of a protein-protein complex. Typically, the deuterium labeling and subsequent quenching experiments are performed, followed by enzymatic digestion, peptide separation, and MS analysis.

Prior to epitope mapping experiments, non-deuteriated experiments were carried out to generate a list of common peptides for recombinant human CD40 monomer (10 μM), and protein complexes of CD40 with mAbs 12D6, 5G7, 19G3, and 5F11 (1:1 molar ratio). In the HDX-MS experiment, 5 μL of each sample (CD40 or CD40 with mAbs 12D6, 5G7, 19G3, and 5F11 respectively) was diluted into 55 μL of D20 buffer (10 mM phosphate buffer, D20, pH7.0) to start the labeling reactions. The reactions were carried out for different periods of time: 20 sec, 1 min, 10 min and 240 min. By the end of each labeling reaction period, the reaction was quenched by adding quenching buffer (100 mM phosphate buffer with 4M GdnCl and 0.4M TCEP, pH 2.5, 1:1, v/v) and 50 μL of quenched sample was injected into Waters HDX-MS system for analysis. The deuterium uptake levels of common peptic peptides were monitored in the absence/presence of CD40 mAbs. The obtained sequence coverage was 82%.

The HDX epitopes for anti-CD40 mAbs 12D6, 5G7, 19G3, and 5F11 are provided at Table 7. Antibody 12D6 protected two peptides, one of which (residues 11-28) includes a portion of the signal sequence and is thus unlikely to be physiologically relevant per se, but indicates that 12D6 makes contacts in the region 21-28 that are not made by mAbs 5G7 and 19G3.

TABLE 7
HDX Epitopes

| | CD40 | |
|---|---|---|
| | residues | |
| | (SEQ ID | |
| Clone | NO: 1) | Sequence |
| 12D6 | 11-28 | WGCLLTAVHPEPPTACRE |
| 12D6 | 21-35 | EPPTACREKQYLINS |
| 5G7 | 21-35 | EPPTACREKQYLINS |
| 19G3 | 21-35 | EPPTACREKQYLINS |
| 5F11 | 58-66 | ECLPCGESE |

### Example 6

### Epitope Mapping by Yeast Display

The epitopes for selected chimeric or humanized anti-huCD40 antibodies of the present invention are determined by displaying randomly mutagenized huCD40 extracellular region variants on yeast, and sorting these yeast based on their failure to bind to particular antibodies. Selected yeast cells that fail to bind are amplified and subjected to additional rounds of selection based on their inability to bind to particular chimeric or humanized forms of the antibodies of the present invention. See, e.g., Chao et al. (2004) J Mol. Biol. 342:539. Sequences for huCD40 variants are determined for the resulting yeast and analyzed for the effects of each residue on antibody binding. The binding epitope for the antibodies of the present invention is determined as the loci within the huCD40 sequence where single amino acid mutations disrupt binding to the anti-huCD40 antibodies of the present invention.

Briefly, error-prone PCR is used to clone human CD40-encoding DNA into constructs allowing expression of the huCD40 variants as the amino-terminal portions of fusion proteins further comprising a c-myc tag sequence and yeast cell wall protein Aga1p. Such constructs, when expressed in yeast (Saccharomyces cerevisiae), display the variant huCD40 polypeptides on the surface of yeast cells, anchored to the cell surface by the Agalp polypeptide. The c-myc tag can optionally be used as a positive control for display of huCD40 fusion proteins on a given yeast cell. Yeast cells are sorted by FACS, and those that express as properly folded huCD40-fusion proteins (as determined by binding of a control mouse anti-huCD40 antibody detected by an allophycocyanin (APC)-labeled goat anti-mouse IgG secondary), but do not bind to the antibodies of the present invention (as determined by detection with a phycoerythrin (PE) labeled goat anti-human IgG as a secondary), are pooled, amplified, and used in subsequent rounds of selection. The huCD40 sequence is determined for constructs from yeast remaining after several rounds of selection. Control experiments without anti-huCD40 antibody selection are performed to confirm good mutant coverage at each position along the huCD40 sequence, and provide a baseline for normalizing the results obtained with the selected libraries.

### Example 7

Agonist Activity of anti-CD40 Antibodies

The effect of Fc sequence variation on the agonist activity of selected anti-CD40 antibodies of the present invention was assessed by measuring activation of immature dendritic cells (DC). Experiments were performed with anti-CD40 mAb 12D6-24 constructs having human IgG1f (control), SE, SELF, P238D, V4, V8 and V12 Fc sequences. Human Monocytes (CD14⁺) were isolated from healthy normal donors using plastic adherence or human CD14-micro beads (Miltenyi Biotec). Monocytes were cultured with 100 ng/mL GM-CSF (Miltenyi Biotec) and 100 ng/mL IL-4 (Miltenyi Biotec). Half of the medium was removed and replenished on day 2 and day 5. Immature dendritic cells were harvested at day 6-7. DC from two donors were incubated with the indicated concentration of antibodies overnight at 37° C. Cell culture supernatants were collected and assayed for human-IL-6 production (Cisbio). See FIGS. 3A and 3B.

Control human IgG1f antibodies did not induce IL-6 secretion when used at up to 100 nM, and the P238D variant induced only weakly. In contrast, SE, SELF, V9 and V12 variants all dramatically enhanced IL-6 secretion, with V8 and V4 exhibiting intermediate effects. These results roughly correlate with binding affinity for FcγRIIb, and confirm that use of the proper Fc sequence variant can result in anti-CD40 antibodies with enhanced agonist activity.

In addition, the differences in agonist activity between antibodies having 8E8, 5G7, 12D6, 19G3 and 5F11 variable regions were evaluated in experiments using chimeric anti-CD40 mAb constructs having a common human IgG1f V12 constant domain. Activation was measured on immature dendritic cells in vitro (isolated as described in the preceding paragraph) by plating cells in a 96 well plate, adding antibodies as indicated, and incubating overnight at 37° C. Cells were then harvested and stained with a fluorescent anti-CD54 antibody, which was detected by fluorescence activated cell sorting (FACS). See FIG. 4.

Control human IgG1f antibody did not significantly cause CD54 upregulation, whereas 12D6 caused dramatic CD54 upregulation. Antibodies 19G3 and 8G8 were somewhat less effective than 12D6, and antibodies 5G7 and 5F11 were similar to each other and exhibited relatively low stimulation. The activation results do not necessarily correlate with binding affinity for CD40, since antibody 5F11 has nearly the highest binding affinity and yet is a weak agonist, whereas antibody 19G3 is the opposite. Regardless of the reason for the differences, these results confirm that use of the proper antigen binding domain sequence is important in obtaining anti-CD40 antibodies with enhanced agonist activity.

### Example 8

### Remediation of DG Isomerization

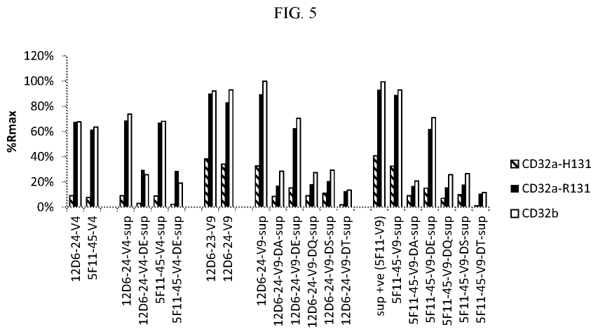

Antibodies of the present invention having V4 and V9 Fc variant sequences exhibit unacceptable levels of DG isomerization at position D270. Such isomerization is undesirable because it leads to product heterogeneity and potentially reduced efficacy. To reduce such isomerization, the aspartic acid residue at position 270 of V4 Fc was changed to glutamic acid (D270E), and position 270 of V9 Fc was changed to alanine, glutamic acid, glutamine, serine and threonine (D270A, D270E, D270Q, D270S, D270T). Supernatants were obtained from cells expressing these antibodies and assayed for binding to hCD32/FcγRII. Selected purified antibodies without mutations at position 270 were used as controls. These experiments were performed on antibodies with variable domains from mAbs 12D6-24 and 5F11-45. Results are provided at FIG. 5.

For both V4 and V9 Fc variants, the D270E substitution led to a modest decrease in receptor binding. Other V9 Fc variants tested (D270A, D270Q, D270S, D270T) led to a greater decrease in receptor binding. Results were independent of whether the antibody was 12D6 or 5F11. In all cases the relative (rank order) binding to the three receptors tested (FcγRIIa-H131, FcγRIIa-R131, and FcγRIIb) remained similar, with roughly equivalent binding to FcγRIIa-R131 and FcγRIIb, and significantly weaker binding to FcγRIIa-H131.

For both V4 abd V9 variants, the D270E substitution retained acceptably high FcγR affinity, and maintained the specificity for FcγRIIb that is desired for enhancing the agonistic activity of the anti-CD40 antibodies of the present invention.

TABLE 8
Summary of Sequence Listing

| SEQ ID | Description |
|---|---|
| 1 | Human CD40 (NP_001241) |
| 2 | Human CD40L-gp39 (NP_000065.1) |
| 3 | 12D6 Chimeric Heavy Chain |
| 4 | 12D6 Chimeric Light Chain |
| 5 | 12D6-03 Heavy Chain |
| 6 | 12D6-03 Light Chain |
| 7 | 12D6-22 Heavy Chain |
| 8 | 12D6-22 V9 Heavy Chain |
| 9 | 12D6-22/12D6-24 Light Chain |
| 10 | 12D6-23 Heavy Chain |
| 11 | 12D6-23 Light Chain |
| 12 | 12D6-24 Heavy Chain |
| 13 | 12D6-24 P238D Heavy Chain |
| 14 | 12D6-24 SE Heavy Chain |
| 15 | 12D6-24 SELF Heavy Chain |
| 16 | 12D6-24 V4 Heavy Chain |
| 17 | 12D6-24 V4 D270E Heavy Chain |
| 18 | 12D6-24 V8 Heavy Chain |
| 19 | 12D6-24 V9 Heavy Chain |
| 20 | 12D6-24 V9 D270E Heavy Chain |
| 21 | 12D6-24 V11 Heavy Chain |
| 22 | 12D6-24 V12 Heavy Chain |
| 23 | 5F11 Chimeric Heavy Chain |
| 24 | 5F11 Chimeric Light Chain |
| 25 | 5F11-17 Heavy Chain |
| 26 | 5F11-17 Light Chain |
| 27 | 5F11-23 Heavy Chain |
| 28 | 5F11-23 Light Chain |
| 29 | 5F11-45 Heavy Chain |
| 30 | 5F11-45 Light Chain |
| 31 | 5F11-45 SE Heavy Chain |
| 32 | 5F11-45 SELF Heavy Chain |
| 33 | 5F11-45 V4 Heavy Chain |
| 34 | 5F11-45 D270E V4 Heavy Chain |
| 35 | 5F11-45 V8 Heavy Chain |
| 36 | 5F11-45 V9 Heavy Chain |
| 37 | 5F11-45 V9 D270E Heavy Chain |
| 38 | 5F11-45 V11 Heavy Chain |
| 39 | 5F11-45 V12 Heavy Chain |
| 40 | 8E8 Chimeric Heavy Chain |
| 41 | 8E8 Chimeric Light Chain |
| 42 | 8E8-56 Heavy Chain |
| 43 | 8E8-56 Light Chain |
| 44 | 8E8-62 Heavy Chain |
| 45 | 8E8-62 Light Chain |
| 46 | 8E8-67 Heavy Chain |
| 47 | 8E8-67 Light Chain |
| 48 | 8E8-70 Heavy Chain |
| 49 | 8E8-70 Light Chain |
| 50 | 8E8-71 Heavy Chain |
| 51 | 8E8-71 Light Chain |
| 52 | 5G7 Chimeric Heavy Chain |
| 53 | 5G7 Chimeric Light Chain |
| 54 | 5G7-22 Heavy Chain |
| 55 | 5G7-22 Light Chain |
| 56 | 5G7-25 Heavy Chain |
| 57 | 5G7-25 Light Chain |
| 58 | 19G3 Chimeric Heavy Chain |
| 59 | 19G3 Chimeric Light Chain |
| 60 | 19G3-11 Heavy Chain |
| 61 | 19G3-11 V9 |
| 62 | 19G3-11 Light Chain |
| 63 | 19G3-22 Heavy Chain |
| 64 | 19G3-22 Light Chain |
| 65 | Human Constant Domain IgG1f |
| 66 | SE Constant Domain |
| 67 | SELF Constant Domain |
| 68 | P238D Constant Domain |
| 69 | V4 Constant Domain |
| 70 | V4 D270E Constant Domain |
| 71 | V7 Constant Domain |
| 72 | V8 Constant Domain |
| 73 | V9 Constant Domain |
| 74 | V9 D270E Constant Domain |
| 75 | V11 Constant Domain |
| 76 | V12 Constant Domain |
| 77 | Light Chain Kappa Constant Domain |
| 78 | Signal Sequence |

The Sequence Listing provides the sequences of the mature heavy and light chains (i.e., sequences do not include signal peptides). A signal sequence for production of the antibodies of the present invention, for example in human cells, is provided at SEQ ID NO: 78.

### EQUIVALENTS

Those skilled in the art will recognize, or be able to ascertain using no more than routine experimentation, many equivalents of the specific embodiments disclosed herein. Such equivalents are intended to be encompassed by the following claims.

We claim:

1. An isolated antibody, or antigen binding portion thereof, that specifically binds to human CD40 and competes for binding to human CD40 in a cross-blocking assay with one or more of antibodies selected from the group consisting of 12D6 (SEQ ID NOs: 3 and 4), 5F11 (SEQ ID NOs: 23 and 24), 8E8 (SEQ ID NOs: 40 and 41), 5G7 (SEQ ID NOs: 52 and 53), and 19G3 (SEQ ID NOs: 58 and 59).

2. The isolated antibody or antigen binding portion thereof of claim 1, wherein the competition in a cross-blocking assay comprises the ability to reduce binding of the selected antibody to human CD40 in a competition ELISA by at least 20% when used at an equal molar concentration with the selected antibody.

3. An isolated antibody, or antigen binding portion thereof, that specifically binds to human CD40 at:
a. an epitope comprising or consisting of the sequence EPPTACREKQYLINS (residues 21-35 of SEQ ID NO: 1) (antibodies 12D6, 5G7 and 19G3); or
b. an epitope comprising or consisting of the sequence ECLPCGESE (residues 58-66 of SEQ ID NO: 1) (antibody 5F11).

4. An isolated antibody, or antigen binding portion thereof, that specifically binds to human CD40 comprising:
a) heavy chain CDR sequences derived at least in part from murine V region germline VH1-39_01 and J region germline IGHJ4 and
light chain CDR sequences derived at least in part from murine V region germline VK1-110_01 and J region germline IGKJ1 (12D6);
b) heavy chain CDR sequences derived at least in part from murine V region germline VH1-4_02 and J region germline IGHJ3 and
light chain CDR sequences derived at least in part from murine V region germline VK3-5_01 and J region germline IGKJ5 (5F11);
c) heavy chain CDR sequences derived at least in part from murine V region germline VH1-80_01 and J region germline IGHJ2 and
light chain CDR sequences derived at least in part from murine V region germline VK1-110_01 and J region germline IGKJ2 (8E8);
d) heavy chain CDR sequences derived at least in part from murine V region germline VH1-18_01 and J region germline IGHJ4 and
light chain CDR sequences derived at least in part from murine V region germline VK10⁻⁹⁶_01 and J region germline IGKJ2 (5G7); or
e) heavy chain CDR sequences derived at least in part from murine V region germline VH5-9-4_01 and J region germline IGHJ3 and
light chain CDR sequences derived at least in part from murine V region germline VK1-117_01 and J region germline IGKJ2 (19G3).

5. The isolated antibody, or antigen binding portion thereof of claim 1 comprising a heavy chain and a light chain, wherein the heavy chain comprises CDRH1, CDRH2 and CDRH3 sequences and the light chain comprises CDRL1, CDRL2 and CDRL3 sequences selected from the group consisting of:
a) the CDRs of antibody 12D6-03 wherein CDRH1, CDRH2 and CDRH3 comprise residues 31-35, 50-66 and 99-108, respectively, of SEQ ID NO:5 and CDRL1, CDRL2 and CDRL3 comprise residues 24-39, 55-61 and 94-102, respectively, of SEQ ID NO:6;
b) the CDRs of antibody 12D6-22 wherein CDRH1, CDRH2 and CDRH3 comprise residues 31-35, 50-66 and 99-108, respectively, of SEQ ID NO:7 and CDRL1, CDRL2 and CDRL3 comprise residues 24-39, 55-61 and 94-102, respectively, of SEQ ID NO:9;
c) the CDRs of antibody 12D6-23 wherein CDRH1, CDRH2 and CDRH3 comprise residues 31-35, 50-66 and 99-108, respectively, of SEQ ID NO:10 and CDRL1, CDRL2 and CDRL3 comprise residues 24-39, 55-61 and 94-102, respectively, of SEQ ID NO:11;
d) the CDRs of antibody 12D6-24 wherein CDRH1, CDRH2 and CDRH3 comprise residues 31-35, 50-66 and 99-108, respectively, of SEQ ID NO:12 and CDRL1, CDRL2 and CDRL3 comprise residues 24-39, 55-61 and 94-102, respectively, of SEQ ID NO:9;
e) the CDRs of antibody 5F11-17 wherein CDRH1, CDRH2 and CDRH3 comprise residues 31-35, 50-66 and 99-106, respectively, of SEQ ID NO:25 and CDRL1, CDRL2 and CDRL3 comprise residues 24-38, 54-60 and 93-101, respectively, of SEQ ID NO:26;
f) the CDRs of antibody 5F11-23 wherein CDRH1, CDRH2 and CDRH3 comprise residues 31-35, 50-66 and 99-106, respectively, of SEQ ID NO:27 and CDRL1, CDRL2 and CDRL3 comprise residues 24-38, 54-60 and 93-101, respectively, of SEQ ID NO:28;
g) the CDRs of antibody 5F11-45 wherein CDRH1, CDRH2 and CDRH3 comprise residues 31-35, 50-66 and 99-106, respectively, of SEQ ID NO:29 and CDRL1, CDRL2 and CDRL3 comprise residues 24-38, 54-60 and 93-101, respectively, of SEQ ID NO:30;
h) the CDRs of antibody 8E8-56 wherein CDRH1, CDRH2 and CDRH3 comprise residues 31-35, 50-66 and 99-111, respectively, of SEQ ID NO:42 and CDRL1, CDRL2 and CDRL3 comprise residues 24-39, 55-61 and 94-102, respectively, of SEQ ID NO:43;
i) the CDRs of antibody 8E8-62 wherein CDRH1, CDRH2 and CDRH3 comprise residues 31-35, 50-66 and 99-111, respectively, of SEQ ID NO:44 and CDRL1, CDRL2 and CDRL3 comprise residues 24-39, 55-61 and 94-102, respectively, of SEQ ID NO:45;
j) the CDRs of antibody 8E8-67 wherein CDRH1, CDRH2 and CDRH3 comprise residues 31-35, 50-66 and 99-111, respectively, of SEQ ID NO:46 and CDRL1, CDRL2 and CDRL3 comprise residues 24-39, 55-61 and 94-102, respectively, of SEQ ID NO:47;
k) the CDRs of antibody 8E8-70 wherein CDRH1, CDRH2 and CDRH3 comprise residues 31-35, 50-66 and 99-111, respectively, of SEQ ID NO:48 and CDRL1, CDRL2 and CDRL3 comprise residues 24-39, 55-61 and 94-102, respectively, of SEQ ID NO:49;
1) the CDRs of antibody 8E8-71 wherein CDRH1, CDRH2 and CDRH3 comprise residues 31-35, 50-66 and 99-111, respectively, of SEQ ID NO:50 and CDRL1, CDRL2 and CDRL3 comprise residues 24-39, 55-61 and 94-102, respectively, of SEQ ID NO:51;
m) the CDRs of antibody 5G7-22 wherein CDRH1, CDRH2 and CDRH3 comprise residues 31-35, 50-66 and 99-102, respectively, of SEQ ID NO:54 and CDRL1, CDRL2 and CDRL3 comprise residues 24-34, 50-56 and 89-97, respectively, of SEQ ID NO:55;
n) the CDRs of antibody 5G7-25 wherein CDRH1, CDRH2 and CDRH3 comprise residues 31-35, 50-66 and 99-102, respectively, of SEQ ID NO:56 and CDRL1, CDRL2 and CDRL3 comprise residues 24-34, 50-56 and 89-97, respectively, of SEQ ID NO:57;
o) the CDRs of antibody 19G3-11 wherein CDRH1, CDRH2 and CDRH3 comprise residues 31-35, 50-66 and 99-101, respectively, of SEQ ID NO:60 and CDRL1, CDRL2 and CDRL3 comprise residues 24-39, 55-61 and 94-102, respectively, of SEQ ID NO:62; and
p) the CDRs of antibody 19G3-22 wherein CDRH1, CDRH2 and CDRH3 comprise residues 31-35, 50-66 and 99-101, respectively, of SEQ ID NO:63 and CDRL1, CDRL2 and CDRL3 comprise residues 24-39, 55-61 and 94-102, respectively, of SEQ ID NO:64.

6. The antibody of claim 5 comprising heavy and light chain variable domain sequences selected from the groups consisting of:
a) the heavy and light chain variable regions of antibody 12D6-03 comprising residues 1-119 and 1-112 of SEQ ID NO:5 and SEQ ID NO:6, respectively;
b) the heavy and light chain variable regions of antibody 12D6-22 comprising residues 1-119 and 1-112 of SEQ ID NO:7 and SEQ ID NO:9, respectively;
c) the heavy and light chain variable regions of antibody 12D6-23 comprising residues 1-119 and 1-112 of SEQ ID NO:10 and SEQ ID NO:11, respectively;
d) the heavy and light chain variable regions of antibody 12D6-24 comprising residues 1-119 and 1-112 of SEQ ID NO:12 and SEQ ID NO:9, respectively;
e) the heavy and light chain variable regions of antibody 5F11-17 comprising residues 1-117 and 1-111 of SEQ ID NO:25 and SEQ ID NO:26, respectively;
f) the heavy and light chain variable regions of antibody 5F11-23 comprising residues 1-117 and 1-111 of SEQ ID NO:27 and SEQ ID NO:28, respectively;
g) the heavy and light chain variable regions of antibody 5F11-45 comprising residues 1-117 and 1-111 of SEQ ID NO:29 and SEQ ID NO:30, respectively;
h) the heavy and light chain variable regions of antibody 8E8-56 comprising residues 1-122 and 1-112 of SEQ ID NO:42 and SEQ ID NO:43, respectively;
i) the heavy and light chain variable regions of antibody 8E8-62 comprising residues 1-122 and 1-112 of SEQ ID NO:44 and SEQ ID NO:45, respectively;
j) the heavy and light chain variable regions of antibody 8E8-67 comprising residues 1-122 and 1-112 of SEQ ID NO:46 and SEQ ID NO:47, respectively;
k) the heavy and light chain variable regions of antibody 8E8-70 comprising residues 1-122 and 1-112 of SEQ ID NO:45 and SEQ ID NO:49, respectively;
l) the heavy and light chain variable regions of antibody 8E8-71 comprising residues 1-122 and 1-112 of SEQ ID NO:50 and SEQ ID NO:51, respectively;
m) the heavy and light chain variable regions of antibody 5G7-22 comprising residues 1-113 and 1-107 of SEQ ID NO:54 and SEQ ID NO:55, respectively;
n) the heavy and light chain variable regions of antibody 5G7-25 comprising residues 1-113 and 1-107 of SEQ ID NO:56 and SEQ ID NO:57, respectively;
o) the heavy and light chain variable regions of antibody 19G3-11 comprising residues 1-112 and 1-112 of SEQ ID NO:60 and SEQ ID NO:62, respectively; and
p) the heavy and light chain variable regions of antibody 19G3-22 comprising residues 1-112 and 1-112 of SEQ ID NO:63 and SEQ ID NO:64, respectively.

7. A nucleic acid encoding the heavy and/or light chain variable region of the antibody, or antigen binding portion thereof, of claim 1.

8. An expression vector comprising the nucleic acid molecule of claim 7.

9. A cell transformed with an expression vector of claim 8.

10. A method of preparing an anti-human CD40 antibody, or antigen binding portion thereof, comprising:
a) expressing the antibody, or antigen binding portion thereof, in the cell of claim 9; and
b) isolating the antibody, or antigen binding portion thereof, from the cell.

11. A pharmaceutical composition comprising:
a) the antibody, or antigen binding portion thereof, of claim 1; and
b) a carrier.

12. A method of stimulating an immune response in a subject comprising administering to the subject the pharmaceutical composition of claim 11.

13. The method of claim 12, wherein the subject has a tumor and an immune response against the tumor is stimulated.

14. The method of claim 12, wherein the subject has a chronic viral infection and an immune response against the viral infection is stimulated.

15. A method of treating cancer comprising administering to a subject in need thereof a therapeutically effective amount of the pharmaceutical composition of claim 11.

16. The method of claim 15, wherein the cancer is selected from the group consisting of: bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer.

17. A method of treating a chronic viral infection comprising administering to a subject in need thereof a therapeutically effective amount of the pharmaceutical composition of claim 11.
